(19) United States
(12) Patent Application Publication
(10) Pub. No.: US 2024/0307383 A1
(43) Pub. Date: Sep. 19, 2024

(54) COMBINATION OF ZIBOTENTAN AND DAPAGLIFLOZIN FOR THE TREATMENT OF PROTEINURIA CHRONIC KIDNEY DISEASE

(71) Applicant: AstraZeneca AB, Södertälje (SE)

(72) Inventors: Philip Ambery, Södertälje (SE); Peter Greasley, Södertälje (SE); Anne-Kristina Mercier, Södertälje (SE); Mikael Sunnåker, Södertälje (SE)

(21) Appl. No.: 18/435,463

(22) Filed: Feb. 7, 2024

Related U.S. Application Data
(60) Provisional application No. 63/601,540, filed on Nov. 21, 2023.
(60) Provisional application No. 63/546,955, filed on Nov. 2, 2023.
(60) Provisional application No. 63/536,599, filed on Sep. 5, 2023.
(60) Provisional application No. 63/483,831, filed on Feb. 8, 2023.

(51) Int. Cl. A61K 31/497; A61K 31/351; A61P 9/12

(52) U.S. Cl. CPC A61K 31/497; A61K 31/351; A61P 9/12

(57) ABSTRACT

Methods for treating high proteinuria chronic kidney disease and/or at least one disease, disorder, or condition associated therewith in patients by the use of a fixed-dose combination of zibotentan and dapagliflozin are disclosed.

### TECHNICAL FIELD

The present disclosure relates to a fixed-dose combination of the endothelin receptor antagonist (ERA) zibotentan and the sodium-dependent glucose cotransporter 2 (SGLT2) inhibitor dapagliflozin for use in treatment of high-proteinuria chronic kidney disease.

### INTRODUCTION

Chronic kidney disease (CKD) is an endothelin-related disease associated with the gradual loss of kidney function. In some instances, CKD may be associated with hypertension or diabetes (Diabetic Kidney Disease, DKD). CKD may be diagnosed by measuring the estimated glomerular filtration rate (eGFR) in the blood, and by measuring albumin and/or protein levels in urine. The severity of CKD is determined by a patient's eGFR levels that correspond with a given stage of disease, ranging from stage 1 (eGFR≥90 mL/min/1.73 m²; normal) to stage 5 (eGFR<15 mL/min/1.73 m²; kidney failure). When diagnosing CKD, albuminuria may also be categorized as an indicator of disease progression. A urine albumin to creatine ratio (UACR) of <30 mg/g is categorized as normal, a UACR between 30-300 mg/g is categorized as moderately increased, and a UACR that is >300 mg/g is categorized as severely increased. There is no cure for, nor way to reverse CKD, and current treatments focus on slowing the progression of kidney damage and controlling symptoms.

Alport syndrome is a hereditary kidney disease caused by COL4A3/4/5 mutation and is the second most common cause of inherited CKD. Alport syndrome manifests in many forms, ranging from hematuria and proteinuria to end-stage kidney disease. There is no cure for Alport syndrome, and current treatments include administration of renin-angiotensin-aldosterone system inhibitors (RAASi).

Anti-neutrophilic cytoplasmic autoantibody (ANCA) vasculitis is an autoimmune disease that causes vasculitis and impacts kidney function. ANCA vasculitis affects kidney function through glomerular inflammation (glomerulonephritis), leading to scarring and/or permanent kidney damage that can ultimately result in end-stage kidney disease. While ANCA vasculitis can be treated by targeting inflammation, treatment can only control the inflammation (induction therapy) and then maintain the disease in remission (maintenance therapy). Current treatments for ANCA vasculitis require the use of immunosuppressives, which can increase an individual's risk of infection.

IgA nephropathy (IgAN), also known as Berger's Disease, is an autoimmune disease caused by the deposition of IgA antibodies in the kidneys. As IgA antibodies accumulate in the small blood vessels of the kidneys, glomeruli become inflamed and damaged. Inflammation and damage in the kidneys can decrease kidney function and result in chronic kidney disease. IgAN may have a slow progression, and progression is more likely in individuals having high blood pressure, large amounts of protein in urine, and/or increased BUN or creatinine levels. Current treatments for IgAN are directed at relieving symptoms and delaying progression of the disease to chronic renal failure, and include ACE inhibitors, corticosteroids and other immunosuppressives, and cholesterol-lowering medications.

Endothelin-1 (ET-1) is a highly potent systemic vasoconstrictor and driver of renal disease progression that is modulated by endothelin A and B receptors (ET_{A} and ET_{B}). In CKD, ET-1 levels increase with UACR and severity of renal functional impairment (Grenda et al., Nephrol Dial Transplant. 2007; 22(12): 3487-3494; Kohan Am J Kidney Dis. 1997; 29(1): 2-26). The pathological effects of ET-1 accumulation, including proteinuria, vasoconstriction and inflammation are thought to be predominantly driven by the ET_{A} receptor (Goddard et al., Circulation. 2004; 109(9): 1186-1193). ET_{A} receptor antagonists have demonstrated kidney protective effects but have side-effects including edema (swelling). In Diabetic Kidney Disease (DKD), short-term treatment with an ET_{A}receptor antagonist has demonstrated a 30% reduction in urinary albumin to creatinine ratio (UACR) (Heerspink et al., Diabetes Obes Metab. 2018; 20(8): 1829-1835; Heerspink et al., Lancet 2019; 393(10184): 1937-1947). However, clinical development of ET_{A} receptor antagonists have been limited due to issues of fluid retention and hospitalization for heart failure (Heerspink et al., Lancet 2019; 393(10184): 1937-1947).

Zibotentan is an ET_{A} receptor antagonist developed for treatment of prostate cancer but was abandoned in 2011 due to insufficient efficacy in Phase 3 and a 17% increase in incidence of peripheral oedema compared to placebo. Zibotentan, also referred to as ZD4054, was described in WO1996040681 along with details on its chemical synthesis, and those teachings are incorporated herein by reference. The specific inhibition of the endothelin A receptor with zibotentan has been reported by Morris et al., British Journal of Cancer (2005), 92, 2148-2152. Zibotentan, N-(3-methoxy-5-methylpyrazin-2-yl)-2-[4-(1,3,4-oxadiazol-2-yl)phenyl]pyridine-3-sulfonamide, has the chemical structure of formula I:

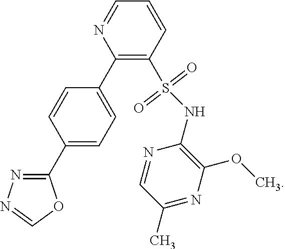 [SMILES: COc1nc(C)cnc1NS(=O)(=O)c1cccnc1-c1ccc(-c2nnco2)cc1]

Sodium-dependent glucose transporter 2 (SGLT-2) inhibitors result in osmotic diuresis and block glucose reabsorption in the kidney, increase glucose excretion, and lower blood glucose concentration. In addition to this well characterized mode of action, SGLT-2 inhibitors reduce blood pressure, decrease vascular stiffness, improve endothelial function, and have anti-inflammatory and anti-fibrotic properties resembling those of endothelin receptor antagonists (H. J. Heerspink et al., Circulation (2016), 134(10): 752-772). Like ET_{A} receptor antagonists, SGLT2 inhibitors have demonstrated efficacy in reducing the progression of DKD (Stephens et al., Diabetes Obes Metab. 2020; 22 Suppl 1:32-45). A side effect associated with the pharmacological effects of SGLT-2 inhibitors is volume depletion/intravascular volume contraction, potentially leading to dehydration, hypovolemia, orthostatic hypotension, or hypotension. Thus, SGLT-2 inhibitors generally induce an increase in hematocrit (Hot) a marker of hemoconcentration and increased blood viscosity, a putative cause of vascular injury in a context of peripheral vascular disease.

Dapagliflozin is a potent, highly selective, and orally active inhibitor of human renal SGLT2 that has been approved to improve glycemic control in adults with type 2 diabetes mellitus (as an adjunct to diet and exercise). Dapagliflozin has been disclosed in WO2003099836 along with details on its chemical synthesis, and those teachings are incorporated herein by reference.

Dapagliflozin, whose IUPAC name is (1S)-1,5-anhydro-1-{4-chloro-3-[(4-ethoxyphenyl)methyl]phenyl}-D-glucitol, has the chemical structure of formula II:

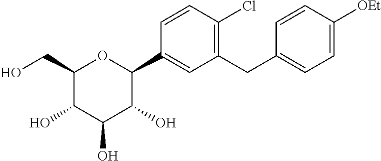 [SMILES: CCOc1ccc(Cc2cc([C@@H]3O[C@H](CO)[C@@H](O)[C@H](O)[C@H]3O)ccc2Cl)cc1]

While approved therapies exist for patients having CKD, there are no approved therapies for patients having high proteinuria CKD characterized by high levels of albuminuria and/or proteinuria. Notably, increased doses of an ET_{A} receptor antagonist that would be required to treat more severe stages of CKD are not well tolerated due to side-effects including edema and risk of congestive heart failure. SGLT2 inhibitors, while effective at treating CKD in many circumstances, leave residual risk in many patients as evidenced by continued proteinuria. For example, a post hoc analysis of the CREDENCE trial showed that while canagliflozin, a SGLT2 inhibitor for treatment of renal events in patients with type 2 diabetes and CKD, was able to reduce UACR levels in the short term, it failed to reduce UACR levels below 300 mg/g in a substantial number of patients and these patients developed kidney events and major adverse cardiovascular events due to residual proteinuria (Oshima et al., J Am Soc Nephrol. (2020); 31(12): 2925-2936).

Thus, there remains a need for improved compounds, compositions, and methods for treating patients with high proteinuria CKD, ANCA vasculitis, Alport syndrome, and IgAN. The present disclosure addresses those unmet needs.

### BRIEF SUMMARY

The present disclosure provides a fixed-dose combination of zibotentan and dapagliflozin for use in the treatment of CKD in a human patient in need of such a treatment, wherein the treatment comprises the administration of zibotentan and dapagliflozin to the human patient in an amount effective to treat the patient's CKD. In some embodiments, the human patient is a CKD patient meeting the criteria described herein.

The present disclosure also provides a method of treating high proteinuria CKD in a human patient in need of such a treatment comprising the separate, sequential, or simultaneous administration of zibotentan and dapagliflozin to the human patient in an amount effective to treat the patient's CKD. In some embodiments, the human patient is a CKD patient meeting the criteria described herein.

The present disclosure also provides the use of a fixed-dose combination of zibotentan and dapagliflozin in the manufacture of a medicament for the treatment of high proteinuria CKD in a human patient in need thereof, wherein the treatment comprises the administration of zibotentan and dapagliflozin to the human patient in an amount effective to treat the patient's CKD. In some embodiments, the human patient is a CKD patient meeting the criteria described herein.

The present disclosure also provides a fixed-dose combination of zibotentan and dapagliflozin for use in the treatment of high proteinuria CKD associated with IgA nephropathy (IgAN) in a human patient in need of such a treatment, wherein the treatment comprises the administration of zibotentan and dapagliflozin to the human patient in an amount effective to treat the patient's IgAN. In some embodiments, the human patient is a high proteinuria CKD patient meeting the criteria described herein.

The present disclosure also provides a method of treating a human patient with high proteinuria CKD associated with IgA nephropathy (IgAN) comprising the separate, sequential, or simultaneous administration of zibotentan and dapagliflozin to the human patient in an amount effective to treat the patient's IgAN. In some embodiments, the human patient is a high proteinuria CKD patient meeting the criteria described herein.

The present disclosure also provides the use of a fixed-dose combination of zibotentan and dapagliflozin in the manufacture of a medicament for the treatment of high proteinuria CKD associated with IgA nephropathy (IgAN) in a human patient in need thereof, wherein the treatment comprises the administration of zibotentan and dapagliflozin to the human patient in an amount effective to treat the patient's IgAN. In some embodiments, the human patient is a high proteinuria CKD patient meeting the criteria described herein.

The present disclosure also provides a fixed-dose combination of zibotentan and dapagliflozin for use in the treatment of IgA nephropathy (IgAN) in a human patient having biopsy-confirmed IgAN in need of such a treatment, wherein the treatment comprises the administration of zibotentan and dapagliflozin to the human patient in an amount effective to treat the patient's IgAN. In some embodiments, the human patient is a high proteinuria CKD patient meeting the criteria described herein.

The present disclosure also provides a method of treating a human patient with biopsy-confirmed IgA nephropathy (IgAN) comprising the separate, sequential, or simultaneous administration of zibotentan and dapagliflozin to the human patient in an amount effective to treat the patient's IgAN. In some embodiments, the human patient is a high proteinuria CKD patient meeting the criteria described herein.

The present disclosure also provides the use of a fixed-dose combination of zibotentan and dapagliflozin in the manufacture of a medicament for the treatment of IgA nephropathy (IgAN) in a human patient having biopsy-confirmed IgAN in need thereof, wherein the treatment comprises the administration of zibotentan and dapagliflozin to the human patient in an amount effective to treat the patient's IgAN. In some embodiments, the human patient is a high proteinuria CKD patient meeting the criteria described herein.

The present disclosure also provides a fixed-dose combination of zibotentan and dapagliflozin for use in reducing proteinuria in a human patient having biopsy-confirmed IgA nephropathy in need of such a treatment, wherein the treatment comprises the administration of zibotentan and dapagliflozin to the human patient in an amount effective to reduce the patient's proteinuria. In some embodiments, the human patient is a high proteinuria CKD patient meeting the criteria described herein.

The present disclosure also provides a method of reducing proteinuria in a human patient with biopsy-confirmed IgA nephropathy comprising the separate, sequential, or simultaneous administration of zibotentan and dapagliflozin to the human patient in an amount effective to reduce the patient's proteinuria. In some embodiments, the human patient is a high proteinuria CKD patient meeting the criteria described herein.

The present disclosure also provides the use of a fixed-dose combination of zibotentan and dapagliflozin in the manufacture of a medicament for reducing proteinuria in a human patient having biopsy-confirmed IgA nephropathy in need thereof, wherein the treatment comprises the administration of zibotentan and dapagliflozin to the human patient in an amount effective to reduce the patient's proteinuria. In some embodiments, the human patient is a high proteinuria CKD patient meeting the criteria described herein.

The present disclosure also provides a fixed-dose combination of zibotentan and dapagliflozin for use in reducing the rate of kidney function decline in a human patient having biopsy-confirmed IgA nephropathy in need of such a treatment, wherein the treatment comprises the administration of zibotentan and dapagliflozin to the human patient in an amount effective to reduce rate of kidney function decline in the patient. In some embodiments, the human patient is a high proteinuria CKD patient meeting the criteria described herein.

The present disclosure also provides a method of reducing the rate of kidney function decline in a human patient with biopsy-confirmed IgA nephropathy comprising the separate, sequential, or simultaneous administration of zibotentan and dapagliflozin to the human patient in an amount effective to reduce the rate of kidney function decline in the patient. In some embodiments, the human patient is a high proteinuria CKD patient meeting the criteria described herein.

The present disclosure also provides the use of a fixed-dose combination of zibotentan and dapagliflozin in the manufacture of a medicament for reducing the rate of kidney function decline in a human patient having biopsy-confirmed IgA nephropathy in need thereof, wherein the treatment comprises the administration of zibotentan and dapagliflozin to the human patient in an amount effective to reduce the rate of kidney function decline in the patient. In some embodiments, the human patient is a high proteinuria CKD patient meeting the criteria described herein.

The present disclosure also provides a fixed-dose combination of zibotentan and dapagliflozin for use in the treatment of high proteinuria CKD associated with focal segmental glomerulosclerosis (FSGS) in a human patient in need of such a treatment, wherein the treatment comprises the administration of zibotentan and dapagliflozin to the human patient in an amount effective to treat the patient's FSGS. In some embodiments, the human patient is a high proteinuria CKD patient meeting the criteria described herein.

The present disclosure also provides a method of treating a human patient with high proteinuria CKD associated with focal segmental glomerulosclerosis (FSGS) comprising the separate, sequential, or simultaneous administration of zibotentan and dapagliflozin to the human patient in an amount effective to treat the patient's FSGS. In some embodiments, the human patient is a high proteinuria CKD patient meeting the criteria described herein.

The present disclosure also provides the use of a fixed-dose combination of zibotentan and dapagliflozin in the manufacture of a medicament for the treatment of high proteinuria CKD associated with FSGS in a human patient in need thereof, wherein the treatment comprises the administration of zibotentan and dapagliflozin to the human patient in an amount effective to treat the patient's FSGS. In some embodiments, the human patient is a high proteinuria CKD patient meeting the criteria described herein.

The present disclosure also provides a fixed-dose combination of zibotentan and dapagliflozin for use in slowing decline in renal function in a human patient in need thereof, wherein the zibotentan and dapagliflozin are administered to the human patient in an amount effective to slow the decline of renal function in the human patient. In some embodiments, the human patient meets the criteria described herein.

The present disclosure also provides a method of slowing decline in renal function in a human patient in need thereof, comprising the separate, sequential, or simultaneous administration of zibotentan and dapagliflozin to the human patient in an amount effective to slow the patient's decline in renal function. In some embodiments, the human patient meets the criteria described herein.

The present disclosure also provides the use of a fixed-dose combination of zibotentan and dapagliflozin in the manufacture of a medicament for slowing decline in renal function in a human patient in need thereof, wherein the zibotentan and dapagliflozin are administered to the human patient in an amount effective to slow the patient's decline in renal function. In some embodiments, the human patient meets the criteria described herein.

The present disclosure also provides a fixed-dose combination of zibotentan and dapagliflozin for use in reducing proteinuria in a human patient, wherein the zibotentan and dapagliflozin are administered to the human patient in an amount effective to reduce the patient's proteinuria. In some embodiments, the human patient meets the criteria described herein.

The present disclosure also provides a method of reducing proteinuria in a human patient in need thereof, comprising the separate, sequential, or simultaneous administration of zibotentan and dapagliflozin to the human patient in an amount effective to reduce the patient's proteinuria. In some embodiments, the human patient meets the criteria described herein.

The present disclosure also provides the use of a fixed-dose combination of zibotentan and dapagliflozin in the manufacture of a medicament for reducing proteinuria in a human patient in need thereof, wherein the zibotentan and dapagliflozin are administered to the human patient in an amount effective to reduce the patient's proteinuria. In some embodiments, the human patient meets the criteria described herein.

The present disclosure also provides a fixed-dose combination of zibotentan and dapagliflozin for use in reducing albuminuria in a human patient in need thereof, wherein the zibotentan and dapagliflozin are administered to the human patient in an amount effective to reduce the patient's albuminuria. In some embodiments, the human patient meets the criteria described herein.

The present disclosure also provides a method of reducing albuminuria in a human patient in need thereof, comprising the separate, sequential, or simultaneous administration of zibotentan and dapagliflozin to the human patient in an amount effective to reduce the patient's albuminuria. In some embodiments, the human patient meets the criteria described herein.

The present disclosure also provides the use of a fixed-dose combination of zibotentan and dapagliflozin in the manufacture of a medicament for reducing albuminuria in a human patient in need thereof, wherein the zibotentan and dapagliflozin are administered to the human patient in an amount effective to reduce the patient's albuminuria. In some embodiments, the human patient meets the criteria described herein.

The present disclosure also provides a fixed-dose combination of zibotentan and dapagliflozin for use in reducing the incidence of a composite endpoint of 30% decline in eGFR, reaching end-stage kidney disease (ESKD), or renal death in a human patient in need thereof, wherein the zibotentan and dapagliflozin are administered to the human patient in an amount effective to reduce the patient's incidence of a composite endpoint of 30% decline in eGFR, reaching ESKD, or renal death. In some embodiments, the human patient meets the criteria described herein.

The present disclosure also provides a method of reducing the incidence of a composite endpoint of 30% decline in eGFR, reaching end-stage kidney disease (ESKD), or renal death in a human patient in need thereof, comprising the separate, sequential, or simultaneous administration of zibotentan and dapagliflozin to the human patient in an amount effective to reduce the patient's incidence of a composite endpoint of 30% decline in eGFR, reaching ESKD, or renal death. In some embodiments, the human patient meets the criteria described herein.

The present disclosure also provides the use of a fixed-dose combination of zibotentan and dapagliflozin in the manufacture of a medicament for reducing the incidence of a composite endpoint of 30% decline in eGFR, reaching end-stage kidney disease (ESKD), or renal death in a human patient in need thereof, wherein the zibotentan and dapagliflozin are administered to the human patient in an amount effective to reduce the patient's incidence of a composite endpoint of 30% decline in eGFR, reaching ESKD, or renal death in the human patient. In some embodiments, the human patient meets the criteria described herein.

The present disclosure also provides a fixed-dose combination of zibotentan and dapagliflozin for use reducing the incidence of a composite endpoint of 40% decline in eGFR, reaching end-stage kidney disease (ESKD), or renal death in a human patient in need thereof, wherein the zibotentan and dapagliflozin are administered to the human patient in an amount effective to reduce the patient's incidence of a composite endpoint of 40% decline in eGFR, reaching ESKD, or renal death. In some embodiments, the human patient meets the criteria described herein.

The present disclosure also provides a method of reducing the incidence of a composite endpoint of 40% decline in eGFR, reaching end-stage kidney disease (ESKD), or renal death in a human patient in need thereof, comprising the separate, sequential, or simultaneous administration of zibotentan and dapagliflozin to the human patient in an amount effective to reduce the patient's incidence of a composite endpoint of 40% decline in eGFR, reaching ESKD, or renal death. In some embodiments, the human patient is a CKD patient meeting the criteria described herein.

The present disclosure also provides the use of a fixed-dose combination of zibotentan and dapagliflozin in the manufacture of a medicament for reducing the incidence of a composite endpoint of 40% decline in eGFR, reaching end-stage kidney disease (ESKD), or renal death in a human patient in need thereof, wherein the zibotentan and dapagliflozin are administered to the human patient in an amount effective to reduce the patient's incidence of a composite endpoint of 40% decline in eGFR, reaching ESKD, or renal death. In some embodiments, the human patient meets the criteria described herein.

The present disclosure also provides a fixed-dose combination of zibotentan and dapagliflozin for use in reducing the incidence of a composite endpoint of 57% decline in eGFR, reaching end-stage kidney disease (ESKD), or renal death in a human patient in need thereof, wherein the zibotentan and dapagliflozin are administered to the human patient in an amount effective to reduce the patient's incidence of a composite endpoint of 57% decline in eGFR, reaching ESKD, or renal death. In some embodiments, the human patient meets the criteria described herein.

The present disclosure also provides a method of reducing the incidence of a composite endpoint of 57% decline in eGFR, reaching end-stage kidney disease (ESKD), or renal death in a human patient in need thereof, comprising the separate, sequential, or simultaneous administration of zibotentan and dapagliflozin to the human patient in an amount effective to reduce the patient's incidence of a composite endpoint of 57% decline in eGFR, reaching ESKD, or renal death. In some embodiments, the human patient meets the criteria described herein.

The present disclosure also provides the use of a fixed-dose combination of zibotentan and dapagliflozin in the manufacture of a medicament for reducing the incidence of a composite endpoint of 57% decline in eGFR, reaching end-stage kidney disease (ESKD), or renal death in a human patient in need thereof, wherein the zibotentan and dapagliflozin are administered to the human patient in an amount effective to reduce the patient's incidence of a composite endpoint of 57% decline in eGFR, reaching ESKD, or renal death. In some embodiments, the human patient meets the criteria described herein.

The present disclosure also provides a fixed-dose combination of zibotentan and dapagliflozin for use in preventing hospitalization of heart failure (hHF) in a human patient in need thereof, wherein the zibotentan and dapagliflozin are administered to the human patient in an amount effective to prevent hHF. In some embodiments, the human patient meets the criteria described herein.

The present disclosure also provides a method of preventing hospitalization of heart failure (hHF) in a human patient in need thereof, comprising the separate, sequential, or simultaneous administration of zibotentan and dapagliflozin to the human patient in an amount effective to prevent hHF. In some embodiments, the human patient meets the criteria described herein.

The present disclosure also provides the use of a fixed-dose combination of zibotentan and dapagliflozin in the manufacture of a medicament for preventing hospitalization of heart failure (hHF) in a human patient in need thereof, wherein the zibotentan and dapagliflozin are administered to the human patient in an amount effective to prevent hHF. In some embodiments, the human patient meets the criteria described herein.

The present disclosure also provides a fixed-dose combination of zibotentan and dapagliflozin for use in reducing the risk of hospitalization of heart failure (hHF) in a human patient in need thereof, wherein the zibotentan and dapagliflozin are administered to the human patient in an amount effective to reduce the patient's risk of hHF. In some embodiments, the human patient meets the criteria described herein.

The present disclosure also provides a method of reducing the risk of hospitalization of heart failure (hHF) in a human patient in need thereof, comprising the separate, sequential, or simultaneous administration of zibotentan and dapagliflozin to the human patient in an amount effective to reduce the patient's risk of hHF. In some embodiments, the human patient meets the criteria described herein.

The present disclosure also provides the use of a fixed-dose combination of zibotentan and dapagliflozin in the manufacture of a medicament for reducing the risk of hospitalization of heart failure (hHF) in a human patient in need thereof, wherein the zibotentan and dapagliflozin are administered to the human patient in an amount effective to reduce the patient's risk of hHF. In some embodiments, the human patient meets the criteria described herein.

The present disclosure also provides a fixed-dose combination of zibotentan and dapagliflozin for use in reducing the risk of increased fluid retention in a human patient in need thereof, wherein the zibotentan and dapagliflozin are administered to the human patient in an amount effective to reduce the patient's risk of increased fluid retention. In some embodiments, the human patient meets the criteria described herein.

The present disclosure also provides a method of reducing the risk of increased fluid retention in a human patient in need thereof, comprising the separate, sequential, or simultaneous administration of zibotentan and dapagliflozin to the human patient in an amount effective to reduce the patient's risk of increased fluid retention. In some embodiments, the human patient meets the criteria described herein.

The present disclosure also provides the use of a fixed-dose combination of zibotentan and dapagliflozin in the manufacture of a medicament for reducing the risk of increased fluid retention in a human patient in need thereof, wherein the zibotentan and dapagliflozin are administered to the human patient in an amount effective to reduce the patient's risk of increased fluid retention. In some embodiments, the human patient meets the criteria described herein.

The present disclosure also provides a fixed-dose combination of zibotentan and dapagliflozin for use in preventing an increase in fluid retention in a human patient in need thereof, wherein the zibotentan and dapagliflozin are administered to the human patient in an amount effective to prevent an increase in fluid retention in the patient. In some embodiments, the human patient meets the criteria described herein.

The present disclosure also provides a method of preventing an increase in fluid retention in a human patient in need thereof, comprising the separate, sequential, or simultaneous administration of zibotentan and dapagliflozin to the human patient in an amount effective to prevent an increase in fluid retention in the patient. In some embodiments, the human patient meets the criteria described herein.

The present disclosure also provides the use of a fixed-dose combination of zibotentan and dapagliflozin in the manufacture of a medicament for preventing an increase in fluid retention in a human patient in need thereof, wherein the zibotentan and dapagliflozin are administered to the human patient in an amount effective to prevent an increase in fluid retention in the patient. In some embodiments, the human patient meets the criteria described herein.

The present disclosure also provides a fixed-dose combination of zibotentan and dapagliflozin for use in reducing the risk of increased levels of brain natriuretic peptide (BNP) in a human patient in need thereof, wherein the zibotentan and dapagliflozin are administered to the human patient in an amount effective to reduce the patient's risk of increased levels of BNP. In some embodiments, the human patient meets the criteria described herein.

The present disclosure also provides a method of reducing the risk of increased levels of brain natriuretic peptide (BNP) in a human patient in need thereof, comprising the separate, sequential, or simultaneous administration of zibotentan and dapagliflozin to the human patient in an amount effective to reduce the patient's risk of increased levels of BNP. In some embodiments, the human patient meets the criteria described herein.

The present disclosure also provides the use of a fixed-dose combination of zibotentan and dapagliflozin in the manufacture of a medicament for reducing the risk of increased levels of brain natriuretic peptide (BNP) in a human patient in need thereof, wherein the zibotentan and dapagliflozin are administered to the human patient in an amount effective to reduce the patient's risk of increased levels of BNP. In some embodiments, the human patient meets the criteria described herein.

The present disclosure also provides a fixed-dose combination of zibotentan and dapagliflozin for use in preventing an increase in levels of brain natriuretic peptide (BNP) in a human patient in need thereof, wherein the zibotentan and dapagliflozin are administered to the human patient in an amount effective to prevent an increase in the patient's levels of BNP. In some embodiments, the human patient meets the criteria described herein.

The present disclosure also provides a method of preventing an increase in levels of brain natriuretic peptide (BNP) in a human patient in need thereof, comprising the separate, sequential, or simultaneous administration of zibotentan and dapagliflozin to the human patient in an amount effective to prevent an increase in the patient's levels of BNP. In some embodiments, the human patient meets the criteria described herein.

The present disclosure also provides the use of a fixed-dose combination of zibotentan and dapagliflozin in the manufacture of a medicament for preventing an increase in levels of brain natriuretic peptide (BNP) in a human patient in need thereof, wherein the zibotentan and dapagliflozin are administered to the human patient in an amount effective to prevent an increase in the patient's levels of BNP. In some embodiments, the human patient meets the criteria described herein.

The present disclosure also provides a fixed-dose combination of zibotentan and dapagliflozin for use in reducing the risk of increased body weight in a human patient in need thereof, wherein the zibotentan and dapagliflozin are administered to the human patient in an amount effective to reduce the patient's risk of increased body weight. In some embodiments, the human patient meets the criteria described herein.

The present disclosure also provides a method of reducing the risk of increased body weight in a human patient in need thereof, comprising the separate, sequential, or simultaneous administration of zibotentan and dapagliflozin to the human patient in an amount effective to reduce the patient's risk of increased body weight. In some embodiments, the human patient meets the criteria described herein.

The present disclosure also provides the use of a fixed-dose combination of zibotentan and dapagliflozin in the manufacture of a medicament for reducing the risk of increased body weight in a human patient in need thereof, wherein the zibotentan and dapagliflozin are administered to the human patient in an amount effective to reduce the patient's risk of increased body weight. In some embodiments, the human patient meets the criteria described herein.

The present disclosure also provides a fixed-dose combination of zibotentan and dapagliflozin for use in preventing an increase in body weight in a human patient in need thereof, wherein the zibotentan and dapagliflozin are administered to the human patient in an amount effective to prevent an increase in the patient's body weight. In some embodiments, the human patient meets the criteria described herein.

The present disclosure also provides a method of preventing an increase in body weight in a human patient in need thereof, comprising the separate, sequential, or simultaneous administration of zibotentan and dapagliflozin to the human patient in an amount effective to prevent an increase in the patient's body weight. In some embodiments, the human patient meets the criteria described herein.

The present disclosure also provides the use of a fixed-dose combination of zibotentan and dapagliflozin in the manufacture of a medicament for preventing an increase in body weight in a human patient in need thereof, wherein the zibotentan and dapagliflozin are administered to the human patient in an amount effective to prevent an increase in the patient's body weight. In some embodiments, the human patient meets the criteria described herein.

The present disclosure also provides a fixed-dose combination of zibotentan and dapagliflozin for use in reducing the risk of increased total body water in a human patient in need thereof, wherein the zibotentan and dapagliflozin are administered to the human patient in an amount effective to reduce the patient's risk of increased total body water. In some embodiments, the human patient meets the criteria described herein.

The present disclosure also provides a method of reducing the risk of increased total body water in a human patient in need thereof, comprising the separate, sequential, or simultaneous administration of zibotentan and dapagliflozin to the human patient in an amount effective to reduce the patient's risk of increased total body water. In some embodiments, the human patient meets the criteria described herein.

The present disclosure also provides the use of a fixed-dose combination of zibotentan and dapagliflozin in the manufacture of a medicament for reducing the risk of increased total body water in a human patient in need thereof, wherein the zibotentan and dapagliflozin are administered to the human patient in an amount effective to reduce the patient's risk of increased total body water. In some embodiments, the human patient meets the criteria described herein.

The present disclosure also provides a fixed-dose combination of zibotentan and dapagliflozin for use in preventing an increase in total body water in a human patient in need thereof, wherein the zibotentan and dapagliflozin are administered to the human patient in an amount effective to prevent an increase in the patient's total body water. In some embodiments, the human patient meets the criteria described herein.

The present disclosure also provides a method of preventing an increase in total body water in a human patient in need thereof, comprising the separate, sequential, or simultaneous administration of zibotentan and dapagliflozin to the human patient in an amount effective to prevent an increase in the patient's total body water. In some embodiments, the human patient meets the criteria described herein.

The present disclosure also provides the use of a fixed-dose combination of zibotentan and dapagliflozin in the manufacture of a medicament for preventing an increase in total body water in a human patient in need thereof, wherein the zibotentan and dapagliflozin are administered to the human patient in an amount effective to prevent an increase in the patient's total body water. In some embodiments, the human patient meets the criteria described herein.

The present disclosure also provides a fixed-dose combination of zibotentan and dapagliflozin for use in reducing cholesterol in a human patient in need thereof, wherein the zibotentan and dapagliflozin are administered to the human patient in an amount effective to reduce the patient's cholesterol. In some embodiments, the human patient meets the criteria described herein.

The present disclosure also provides a method of reducing cholesterol in a human patient in need thereof, comprising the separate, sequential, or simultaneous administration of zibotentan and dapagliflozin to the human patient in an amount effective to reduce the patient's cholesterol. In some embodiments, the human patient meets the criteria described herein.

The present disclosure also provides the use of a fixed-dose combination of zibotentan and dapagliflozin in the manufacture of a medicament for reducing cholesterol in a human patient in need thereof, wherein the zibotentan and dapagliflozin are administered to the human patient in an amount effective to reduce the patient's cholesterol. In some embodiments, the human patient meets the criteria described herein.

The present disclosure also provides a fixed-dose combination of zibotentan and dapagliflozin for use in reducing hemoglobin A1c (HbA1c) in a human patient in need thereof, wherein the zibotentan and dapagliflozin are administered to the human patient in an amount effective to reduce the patient's HbA1c. In some embodiments, the human patient meets the criteria described herein.

The present disclosure also provides a method of reducing hemoglobin A1c (HbA1c) in a human patient in need thereof, comprising the separate, sequential, or simultaneous administration of zibotentan and dapagliflozin to the human patient in an amount effective to reduce the patient's HbA1c. In some embodiments, the human patient meets the criteria described herein.

The present disclosure also provides the use of a fixed-dose combination of zibotentan and dapagliflozin in the manufacture of a medicament for reducing hemoglobin A1c (HbA1c) in a human patient in need thereof, wherein the zibotentan and dapagliflozin are administered to the human patient in an amount effective to reduce the patient's HbA1c. In some embodiments, the human patient meets the criteria described herein.

The present disclosure also provides a fixed-dose combination of zibotentan and dapagliflozin for use in reducing the risk of cholesterol elevation in a human patient in need thereof, wherein the zibotentan and dapagliflozin are administered to the human patient in an amount effective to reduce the risk of cholesterol elevation. In some embodiments, the human patient meets the criteria described herein.

The present disclosure also provides a method of reducing the risk of cholesterol elevation in a human patient in need thereof, comprising the separate, sequential, or simultaneous administration of zibotentan and dapagliflozin to the human patient in an amount effective to reduce the risk of cholesterol elevation. In some embodiments, the human patient meets the criteria described herein.

The present disclosure also provides the use of a fixed-dose combination of zibotentan and dapagliflozin in the manufacture of a medicament for reducing the risk of cholesterol elevation in a human patient in need thereof, wherein the zibotentan and dapagliflozin are administered to the human patient in an amount effective to reduce the risk of cholesterol elevation. In some embodiments, the human patient meets the criteria described herein.

The present disclosure also provides a fixed-dose combination of zibotentan and dapagliflozin for use in reducing the risk of hemoglobin A1c (HbA1c) elevation in a human patient in need thereof, wherein the zibotentan and dapagliflozin are administered to the human patient in an amount effective to reduce the risk of HbA1c elevation. In some embodiments, the human patient meets the criteria described herein.

The present disclosure also provides a method of reducing the risk of hemoglobin A1c (HbA1c) elevation in a human patient in need thereof, comprising the separate, sequential, or simultaneous administration of zibotentan and dapagliflozin to the human patient in an amount effective to reduce the risk of HbA1c elevation. In some embodiments, the human patient meets the criteria described herein.

The present disclosure also provides the use of a fixed-dose combination of zibotentan and dapagliflozin in the manufacture of a medicament for reducing the risk of hemoglobin A1c (HbA1c) elevation in a human patient in need thereof, wherein the zibotentan and dapagliflozin are administered to the human patient in an amount effective to reduce the risk of HbA1c elevation. In some embodiments, the human patient meets the criteria described herein.

The present disclosure also provides a fixed-dose combination of zibotentan and dapagliflozin for use in reducing blood pressure in a human patient in need thereof, wherein the zibotentan and dapagliflozin are administered to the human patient in an amount effective to reduce the patient's blood pressure. In some embodiments, the human patient meets the criteria described herein.

The present disclosure also provides a method of reducing blood pressure in a human patient in need thereof, comprising the separate, sequential, or simultaneous administration of zibotentan and dapagliflozin to the human patient in an amount effective to reduce the patient's blood pressure. In some embodiments, the human patient meets the criteria described herein.

The present disclosure also provides the use of a fixed-dose combination of zibotentan and dapagliflozin in the manufacture of a medicament for reducing blood pressure in a human patient in need thereof, wherein the zibotentan and dapagliflozin are administered to the human patient in an amount effective to reduce the patient's blood pressure. In some embodiments, the human patient meets the criteria described herein.

The present disclosure also provides a fixed-dose combination of zibotentan and dapagliflozin for use in reducing the risk of blood pressure elevation in a human patient in need thereof, wherein the zibotentan and dapagliflozin are administered to the human patient in an amount effective to reduce the risk of blood pressure elevation. In some embodiments, the human patient meets the criteria described herein.

The present disclosure also provides a method of reducing the risk of blood pressure elevation in a human patient in need thereof, comprising the separate, sequential, or simultaneous administration of zibotentan and dapagliflozin to the human patient in an amount effective to reduce the risk of blood pressure elevation. In some embodiments, the human patient meets the criteria described herein.

The present disclosure also provides the use of a fixed-dose combination of zibotentan and dapagliflozin in the manufacture of a medicament for reducing the risk of blood pressure elevation in a human patient in need thereof, wherein the zibotentan and dapagliflozin are administered to the human patient in an amount effective to reduce the risk of blood pressure elevation. In some embodiments, the human patient meets the criteria described herein.

The present disclosure also provides a fixed-dose combination of zibotentan and dapagliflozin for use in reducing the incidence of antineutrophilic cytoplasmic autoantibody (ANCA) vasculitis in a human patient in need thereof, wherein the zibotentan and dapagliflozin are administered to the human patient in an amount effective to reduce the incidence of ANCA vasculitis. In some embodiments, the human patient meets the criteria described herein.

The present disclosure also provides a method of reducing the incidence of antineutrophilic cytoplasmic autoantibody (ANCA) vasculitis in a human patient in need thereof, comprising the separate, sequential, or simultaneous administration of zibotentan and dapagliflozin to the human patient in an amount effective to reduce the incidence of ANCA vasculitis. In some embodiments, the human patient meets the criteria described herein.

The present disclosure also provides the use of a fixed-dose combination of zibotentan and dapagliflozin in the manufacture of a medicament for reducing the incidence of antineutrophilic cytoplasmic autoantibody (ANCA) vasculitis in a human patient in need thereof, wherein the zibotentan and dapagliflozin are administered to the human patient in an amount effective to reduce the incidence of ANCA vasculitis. In some embodiments, the human patient meets the criteria described herein.

The present disclosure also provides a fixed-dose combination of zibotentan and dapagliflozin for use in reducing the recurrence of antineutrophilic cytoplasmic autoantibody (ANCA) vasculitis in a human patient in need thereof, wherein the zibotentan and dapagliflozin are administered to the human patient in an amount effective to reduce the recurrence of ANCA vasculitis. In some embodiments, the human patient meets the criteria described herein.

The present disclosure also provides a method of reducing the recurrence of antineutrophilic cytoplasmic autoantibody (ANCA) vasculitis in a human patient in need thereof, comprising the separate, sequential, or simultaneous administration of zibotentan and dapagliflozin to the human patient in an amount effective to reduce the recurrence of ANCA vasculitis. In some embodiments, the human patient meets the criteria described herein.

The present disclosure also provides the use of a fixed-dose combination of zibotentan and dapagliflozin in the manufacture of a medicament for reducing the recurrence of antineutrophilic cytoplasmic autoantibody (ANCA) vasculitis in a human patient in need thereof, wherein the zibotentan and dapagliflozin are administered to the human patient in an amount effective to reduce the recurrence of ANCA vasculitis. In some embodiments, the human patient meets the criteria described herein.

The present disclosure also provides a fixed-dose combination of zibotentan and dapagliflozin for use in reducing the progression of Alport syndrome in a human patient in need thereof, wherein the zibotentan and dapagliflozin are administered to the human patient in an amount effective to reduce the progression of Alport syndrome. In some embodiments, the human patient meets the criteria described herein.

The present disclosure also provides a method of reducing the progression of Alport syndrome in a human patient in need thereof, comprising the separate, sequential, or simultaneous administration of zibotentan and dapagliflozin to the human patient in an amount effective to reduce the progression of Alport syndrome. In some embodiments, the human patient meets the criteria described herein.

The present disclosure also provides the use of a fixed-dose combination of zibotentan and dapagliflozin in the manufacture of a medicament for reducing the progression of Alport syndrome in a human patient in need thereof, wherein the zibotentan and dapagliflozin are administered to the human patient in an amount effective to reduce the progression of Alport syndrome. In some embodiments, the human patient meets the criteria described herein.

The present disclosure also provides a fixed-dose combination of zibotentan and dapagliflozin for use in treating Alport syndrome in a human patient in need thereof, wherein the zibotentan and dapagliflozin are administered to the human patient in an amount effective to treat Alport syndrome. In some embodiments, the human patient meets the criteria described herein.

The present disclosure also provides a method of treating Alport syndrome in a human patient in need thereof, comprising the separate, sequential, or simultaneous administration of zibotentan and dapagliflozin to the human patient in an amount effective to treat Alport syndrome. In some embodiments, the human patient meets the criteria described herein.

The present disclosure also provides the use of a fixed-dose combination of zibotentan and dapagliflozin in the manufacture of a medicament for treating Alport syndrome in a human patient in need thereof, wherein the zibotentan and dapagliflozin are administered to the human patient in an amount effective to treat Alport syndrome. In some embodiments, the human patient meets the criteria described herein.

The present disclosure also provides a fixed-dose combination of zibotentan and dapagliflozin for use in reducing levels of markers of kidney inflammation (nephritis) associated with Alport syndrome in a human patient in need thereof, wherein the zibotentan and dapagliflozin are administered to the human patient in an amount effective to reduce levels of markers of kidney inflammation (nephritis) associated with Alport syndrome. In some embodiments, the human patient meets the criteria described herein.

The present disclosure also provides a method of reducing levels of markers of kidney inflammation (nephritis) associated with Alport syndrome in a human patient in need thereof, comprising the separate, sequential, or simultaneous administration of zibotentan and dapagliflozin to the human patient in an amount effective to reduce levels of markers of kidney inflammation (nephritis) associated with Alport syndrome. In some embodiments, the human patient meets the criteria described herein.

The present disclosure also provides the use of a fixed-dose combination of zibotentan and dapagliflozin in the manufacture of a medicament for reducing levels of markers of kidney inflammation (nephritis) associated with Alport syndrome in a human patient in need thereof, wherein the zibotentan and dapagliflozin are administered to the human patient in an amount effective to reduce levels of markers of kidney inflammation (nephritis) associated with Alport syndrome. In some embodiments, the human patient meets the criteria described herein.

The present disclosure also provides a fixed-dose combination of zibotentan and dapagliflozin for use in reducing the incidence of stroke in a human patient in need thereof, wherein the zibotentan and dapagliflozin are administered to the human patient in an amount effective to reduce the incidence of stroke. In some embodiments, the human patient meets the criteria described herein.

The present disclosure also provides a method of reducing the incidence of stroke in a human patient in need thereof, comprising the separate, sequential, or simultaneous administration of zibotentan and dapagliflozin to the human patient in an amount effective to reduce the incidence of stroke. In some embodiments, the human patient meets the criteria described herein.

The present disclosure also provides the use of a fixed-dose combination of zibotentan and dapagliflozin in the manufacture of a medicament for reducing the incidence of stroke in a human patient in need thereof, wherein the zibotentan and dapagliflozin are administered to the human patient in an amount effective to reduce the incidence of stroke. In some embodiments, the human patient meets the criteria described herein.

The present disclosure also provides a fixed-dose combination of zibotentan and dapagliflozin for use in reducing the risk of stroke in a human patient in need thereof, wherein the zibotentan and dapagliflozin are administered to the human patient in an amount effective to reduce the risk of stroke. In some embodiments, the human patient meets the criteria described herein.

The present disclosure also provides a method of reducing the risk of stroke in a human patient in need thereof, comprising the separate, sequential, or simultaneous administration of zibotentan and dapagliflozin to the human patient in an amount effective to reduce the risk of stroke. In some embodiments, the human patient meets the criteria described herein.

The present disclosure also provides the use of a fixed-dose combination of zibotentan and dapagliflozin in the manufacture of a medicament for reducing the risk of stroke in a human patient in need thereof, wherein the zibotentan and dapagliflozin are administered to the human patient in an amount effective to reduce the risk of stroke. In some embodiments, the human patient meets the criteria described herein.

In embodiments, the patient is naive to a sodium-glucose cotransporter-2 (SGLT2) inhibitor.

In embodiments, the patient has an estimated glomerular filtration rate (eGFR) of 20-90 mL/min/1.73 m².

In embodiments, the patient has a UPCR of 1-1.3 g/g. In embodiments, the patient has a UACR of 700-900 mg/g.

In embodiments, the patient has a UPCR of 1-1.3 g/g and a UACR of 700-900 mg/g.

In some embodiments, the patient has biopsy-confirmed IgA nephropathy.

In embodiments, the fixed-dose combination of zibotentan and dapagliflozin is administered to the patient once per day.

In embodiments, zibotentan is administered at a dose of 0.25 mg to 1.5 mg. In embodiments, zibotentan is administered at a dose of 0.25 mg. In embodiments, zibotentan is administered at a dose of 0.5 mg. In embodiments, zibotentan is administered at a dose of 0.75 mg. In embodiments, zibotentan is administered at a dose of 1.0 mg. In embodiments, zibotentan is administered at a dose of 1.25 mg. In embodiments, zibotentan is administered at a dose of 1.5 mg.

In embodiments, dapagliflozin is administered at a dose of 2.5 mg to 10 mg. In embodiments, dapagliflozin is administered at a dose of 2.5 mg. In embodiments, dapagliflozin is administered at a dose of 5.0 mg. In embodiments, dapagliflozin is administered at a dose of 10.0 mg.

In embodiments, zibotentan is administered at a dose of 0.75 mg and dapagliflozin is administered at a dose of 10 mg. In embodiments, zibotentan is administered at a dose of 0.5 mg and dapagliflozin is administered at a dose of 10 mg. In embodiments, zibotentan is administered at a dose of 0.25 mg and dapagliflozin is administered at a dose of 10 mg.

In some embodiments, wherein the patient has an eGFR 2:45 mL/min/1.73 m², zibotentan is administered at a dose of 0.75 mg and dapagliflozin is administered at a dose of 10 mg. In some embodiments, wherein the patient has an eGFR<45 mL/min/1.73 m², zibotentan is administered at a dose of 0.25 mg and dapagliflozin is administered at a dose of 10 mg. In some embodiments, wherein the patient's eGFR changes to <45 mL/min/1.73 m², the fixed dose combination of zibotentan and dapagliflozin is adjusted so zibotentan is administered at a dose of 0.25 mg and dapagliflozin is administered at a dose of 10 mg. In some embodiments, wherein the patient's eGFR changes to 2:45 mL/min/1.73 m², the fixed dose combination of zibotentan and dapagliflozin is adjusted so zibotentan is administered at a dose of 0.75 mg and dapagliflozin is administered at a dose of 10 mg.

In an embodiment, the method or use of any of the embodiments disclosed herein reduces the patient's UACR to below 300 mg/g. In an embodiment, the method or use of any of the embodiments disclosed herein reduces the patient's UPCR to below 1 g/g. In an embodiment, the method or use of any of the embodiments disclosed herein reduces the patient's UACR to below 300 mg/g and the patient's UPCR to below 1 g/g.

In an embodiment, the method or use of any of the embodiments disclosed herein results in the patient achieving partial remission or remission.

In an embodiment, the method or use of any of the embodiments disclosed herein reduces incidence of stroke and/or acute coronary syndrome in the patient relative to a dosing regimen in which the patient receives dapagliflozin alone.

In any of the above embodiments, the results produced by the methods and uses provided may be relative to a human patient receiving at least one SGLT2-inhibitor (e.g., dapagliflozin, empagliflozin, canagliflozin, etc.) alone or in combination with at least one standard of care CKD agent. In such aspects, the standard of care CKD agent may be an ACE-inhibitor (e.g., captopril, enalapril, and lisinopril) and/or an angiotensin II receptor blocker (ARB) (valsartan, losartan, and irbesartan). In some embodiments, the results are relative to dapagliflozin alone. In some embodiments, the results are relative to dapagliflozin in combination with at least one standard of care CKD agent.

In any of the above embodiments, the results produced by the methods and uses provided may be relative to the patient from baseline. In some embodiments, the methods and uses thereof may be relative to the patient from baseline compared to patients receiving dapagliflozin alone or in combination with at least one standard of care for CKD.

### BRIEF DESCRIPTION OF THE FIGURES

FIG. 1 illustrates the study timeline and study groups set forth in Example 1.

FIG. 2 illustrates the study timeline and study groups set forth in Example 2.

FIG. 3 illustrates the dose-titration grouping set forth in Example 2.

FIG. 4 shows a Kaplan-Meier curve illustrating the impact of the combination of zibotentan and dapagliflozin at two different doses and dapagliflozin monotherapy on fluid events over a 12-week period.

FIG. 5 illustrates the impact of the combination of zibotentan and dapagliflozin at two different doses and dapagliflozin monotherapy on UACR change from baseline levels over a 12-week period.

FIG. 6 illustrates the impact of the combination of zibotentan and dapagliflozin at two different doses and dapagliflozin monotherapy on systolic blood pressure changes from baseline levels over a 12-week period.

FIGS. 7A and 7B depict the impact of the combination of zibotentan and dapagliflozin at two different doses, and dapagliflozin monotherapy, on systolic blood pressure and diastolic blood pressure changes from baseline over a 12-week period in patients having a UACR>700 mg/g.

FIG. 8 depicts the study timeline and groups set forth in Example 4.

### DETAILED DESCRIPTION

As used herein, the terms “about” and “approximately,” when used to modify a numeric value or numeric range, indicate that deviations of up to 10% above and down to 10% below the value or range remain within the intended meaning of the recited value or range. In some embodiments, “about” refers to ±10%. In some embodiments, “about” refers to ±9%. In some embodiments, “about” refers to ±8%. In some embodiments, “about” refers to ±7%. In some embodiments, “about” refers to ±6%. In some embodiments, “about” refers to ±5%. In some embodiments, “about” refers to ±4%. In some embodiments, “about” refers to ±3%. In some embodiments, “about” refers to ±2%. In some embodiments, “about” refers to ±1%. It is understood that wherever aspects are described herein with the language “about” or “approximately” a numeric value or range, otherwise analogous aspects referring to the specific numeric value or range (without “about”) are also provided. It is also understood that wherever aspects are described herein referring to a numeric value or range without the language “about” or “approximately,” otherwise analogous aspects referring to “about” or “approximately” the specific numeric value or range are also provided.

The terms “treating” or “treatment” or “to treat” refer to therapeutic measures that cure, slow down, lessen symptoms of, and/or halt progression of a diagnosed pathologic disease, disorder, or condition. Treatment need not result in a complete cure of the condition; partial inhibition or reduction of the condition being treated is encompassed by this term.

The effectiveness of the compounds of the present disclosure in treating and/or preventing chronic kidney disease (CKD) and/or diseases, disorders, and/or conditions associated therewith can readily be determined by a person of ordinary skill in the relevant art. Determining and adjusting an appropriate dosing regimen (e.g., adjusting the amount of compound per dose and/or number of doses and frequency of dosing) can also readily be performed by a person of ordinary skill in the relevant art. One or any combination of diagnostic methods, including physical examination, assessment and monitoring of clinical symptoms, and performance of analytical tests and methods described herein, may be used for monitoring the health status of the patient.

An effective amount or therapeutically effective amount refers to an amount of at least one compound of the present disclosure or a pharmaceutical composition comprising at least one such compound that, when administered to a patient, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the patient. Patients may generally be monitored for therapeutic effectiveness using assays suitable for the disease, disorder, and/or condition being treated or prevented, which assays will be familiar to those having ordinary skill in the art and are described herein. The level of a compound that is administered to a patient may be monitored by determining the level of the compound (or a metabolite of the compound) in a biological fluid, for example, in the blood, blood fraction (e.g., serum), and/or in the urine, and/or another biological sample from the patient. Any method practiced in the art to detect the compound, or metabolite thereof, may be used to measure the level of the compound during the course of a therapeutic regimen.

The dose of a compound described herein may depend upon the patient's condition, that is, stage of the disease, severity of symptoms caused by the disease, general health status, as well as age, gender, and weight, and other factors apparent to a person of ordinary skill in the medical art. Similarly, the dose of the therapeutic for treating a disease, disorder, and/or condition may be determined according to parameters understood by a person of ordinary skill in the medical art.

As used herein, the terms “subject” and “patient” are used interchangeably to refer to a party receiving a medical treatment. In some aspects, the subject is a human.

As used herein, the term “albuminuria” refers to the presence of albumin in urine, a marker for chronic kidney disease. Albuminuria is present when the urine albumin to creatine ratio (UACR) is above 30 mg/g.

As used herein, the term “proteinuria” refers to the presence of protein in urine, a marker for chronic kidney disease. Proteinuria is present when the urine protein to creatine ratio (UPCR) is above 150 mg/g. In some embodiments, proteinuria is present when the UPCR is above 200 mg/g.

As used herein, the “severity” of chronic kidney disease (CKD) refers to the stage or grade of CKD as defined by the Kidney Disease Improving Global Outcomes (KIDGO) Guidelines. Stage 1 is normal or high eGFR (eGFR>90 mL/min/1.73 m²). Stage 2 is mild CKD (eGFR=60-89 mL/min/1.73 m²). Stage 3A is moderate CKD (eGFR=45-59 mL/min/1.73 m²). Stage 3B is moderate CKD (eGFR=30-44 mL/min/1.73 m²). Stage 4 is severe CKD (eGFR=15-29 mL/min/1.73 m²). Stage 5 is end-stage CKD (eGFR<15 mL/min/1.73 m²).

As used herein, the term “end-stage kidney disease (ESKD)” refers to (i) having a sustained eGFR<15 mL/min/1.73 m², (ii) receiving chronic dialysis treatment, or (iii) receiving a renal transplant. In some embodiments, “sustained” refers to a confirmation of a similar eGFR measurement by a second eGFR test 3 months apart.

As used herein, the term “cholesterol” refers to (i) low-density lipoprotein (LDL) cholesterol, (ii) high-density lipoprotein (HDL) cholesterol, or both.

As used herein, the phrase “not naive to SGLT2 inhibitors” refers to a patient who has (i) previously received SGLT2 inhibitor therapy; and/or (ii) is currently receiving SGLT2 inhibitor therapy.

The terms “administer,” “administering,” “administration,” and the like, as used herein, refer to methods that may be used to enable delivery of a drug, e.g., zibotentan or a pharmaceutically acceptable salt, solvate, mixed solvate, complex, or prodrug thereof and dapagliflozin or a pharmaceutically acceptable salt, solvate, mixed solvate, complex, or prodrug thereof, as described herein. Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa. In some aspects, zibotentan and dapagliflozin are administered orally.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed, is appropriate for the formulation employed, and is compatible with other ingredients of the formulation.

A “sterile” formulation is aseptic or essentially free from living microorganisms and their spores.

As used herein, the term “prodrug” refers to, for example, esters and carbonates that may be converted, for example, under physiological conditions or by solvolysis, to zibotentan or dapagliflozin. Thus, the term prodrug includes metabolic precursors of zibotentan or dapagliflozin that are pharmaceutically acceptable. The term prodrug also includes covalently bonded carriers that release zibotentan or dapagliflozin in vivo when such prodrug is administered to a patient. Non-limiting examples of prodrugs include esters and carbonates.

Various forms of prodrugs are known in the art. For examples of such prodrug derivatives, see: (1) Design of Prodrugs, edited by H. Bundgaard, (Elsevier, 1985) and Methods in Enzymology, Vol. 42, p. 309-396, edited by K. Widder, et al. (Academic Press, 1985); (2) A Textbook of Drug Design and Development, edited by Krogsgaard-Larsen and H. Bundgaard, Chapter 5 “Design and Application of Prodrugs”, by H. Bundgaard p. 113-191 (1991); (3) H. Bundgaard, Advanced Drug Delivery Reviews, 8, 1-38 (1992); (4) H. Bundgaard, et al., Journal of Pharmaceutical Sciences, 77, 285 (1988); and (5) N. Kakeya, et al., Chem Pharm Bull, 32, 692 (1984).

It is understood that wherever aspects are described herein with the language “comprising,” otherwise analogous aspects described in terms of “consisting of” and/or “consisting essentially of” are also provided. In this disclosure, “comprises,” “comprising,” “containing,” and “having” and the like can mean “includes,” “including,” and the like; “consisting essentially of” or “consists essentially” are open-ended, allowing for the presence of more than that which is recited so long as basic or novel characteristics as described herein of that which is recited are not changed by the presence of more than that which is recited, but excludes prior art aspects.

Unless specifically stated or obvious from context, as used herein, the term “or” is understood to be inclusive.

In an aspect, the disclosure herein provides methods of treating high proteinuria chronic kidney disease (CKD) in a human patient comprising administering zibotentan and dapagliflozin to a patient in need thereof.

In an aspect, the disclosure herein provides a fixed-dose combination of zibotentan and dapagliflozin for use in treating high proteinuria chronic kidney disease (CKD) in a human patient.

In an aspect, the disclosure herein provides the use of a fixed-dose combination of zibotentan and dapagliflozin in the manufacture of a medicament for the treatment of high proteinuria chronic kidney disease (CKD) in a human patient, wherein a fixed-dose combination of zibotentan and dapagliflozin is administered.

In embodiments, the chronic kidney disease (CKD) is CKD of stage 1 to 4 as defined by the Kidney Disease Improving Global Outcomes (KIDGO) Guidelines. In embodiments, the CKD is CKD of stages 2-3. In embodiments, the CKD is CKD of stages 3-4. In embodiments, the CKD is CKD of stages 2-4. In embodiments, the CKD is CKD of stage 4. In embodiments, the CKD is CKD of stage 3. In embodiments, the CKD is CKD of stages 3A or 3B. In embodiments, the CKD is CKD of stage 2.

In embodiments, the patient is a CKD human patient with Type 2 diabetes. In embodiments, the patient is a CKD human patient without Type 2 diabetes.

In embodiments, the patient has an estimated glomerular filtration rate (eGFR) of 20-90 mL/min/1.73 m². In some embodiments, the patient has an eGFR of 20-29 mL/min/1.73 m². In some embodiments, the patient has an eGFR of 30-44 mL/min/1.73 m². In some embodiments, the patient has an eGFR of 45-59 mL/min/1.73 m². In some embodiments, the patient has an eGFR of 60-89 mL/min/1.73 m².

In embodiments, the patient has a urinary protein to creatine ratio (UPCR) that is greater than 2.0 g/g. In embodiments, the patient has a UPCR greater than 1.0 g/g. In embodiments, the patient has a UPCR of 1.0-2.0 g/g. In embodiments, the patient has a UPCR of 1.0-1.5 g/g. In embodiments, the patient has a UPCR of 1.0-1.3 g/g.

In embodiments, the patient has a urinary albumin to creatine ratio (UACR) that is greater than 1.5 g/g. In embodiments, the patient has a UACR of greater than 700 mg/g. In embodiments, the patient has a UACR of 700 mg/g to 1.5 g/g. In embodiments, the patient has a UACR of 700 mg/g-1.0 g/g. In embodiments, the patient has a UACR of 700-900 mg/g.

In embodiments, the patient has an eGFR of 20-90 mL/min/1.73 m² and a UACR of greater than 700 mg/g. In embodiments, the patient has an eGFR of 20-90 mL/min/1.73 m² and a UPCR of greater than 1.0 g/g. In embodiments, the patient has a UACR of greater than 700 mg/g and a UPCR of greater than 1.0 g/g. In embodiments, the patient has an eGFR of 20-90 mL/min/1.73 m², a UACR of greater than 700 mg/g, and a UPCR of greater than 1.0 g/g.

In embodiments, the patient has an eGFR of 20-90 mL/min/1.73 m² and a UACR of 700-900 mg/g. In embodiments, the patient has an eGFR of 20-90 mL/min/1.73 m² and a UPCR of 1.0-1.3 g/g. In embodiments, the patient has a UACR of 700-900 mg/g and a UPCR of 1.0-1.3 g/g. In embodiments, the patient has an eGFR of 20-90 mL/min/1.73 m², a UACR of 700-900 mg/g, and a UPCR of 1.0-1.3 g/g.

In embodiments, the patient has Alport syndrome. In embodiments, the patient has high proteinuria (UACR>700 mg/g) and Alport Syndrome. In embodiments, the patient has antineutrophilic cytoplasmic autoantibody (ANCA) vasculitis. In embodiments, the patient has high proteinuria (UACR>700 mg/g) and ANCA vasculitis.

In some embodiments, the patient has IgA nephropathy (IgAN). In some embodiments, the patient has high proteinuria (UACR>700 mg/g and/or UPCR>1.0 g/g) and IgAN. In some embodiments, the patient has an eGFR>30 mL/min/1.73 m², high proteinuria (UACR>700 mg/g and/or UPCR>1.0 g/g), and IgAN. In some embodiments, the patient's diagnosis of IgAN is biopsy-confirmed. In some embodiments, the patient having IgAN is at risk of rapid disease progression.

In embodiments, the patient is naive to a sodium-glucose cotransporter 2 (SGLT2) inhibitor. In some embodiments wherein the patient is naïve to a SGLT2 inhibitor, the patient receives dapagliflozin (run-in) prior to receiving the fixed-dose combination of zibotentan and dapagliflozin. In some embodiments wherein the patient is naïve to a SGLT2 inhibitor, the patient receives a 4-week run-in of dapagliflozin prior to receiving the fixed-dose combination of zibotentan and dapagliflozin. In embodiments, the patient is not naive to a SGLT2 inhibitor.

In embodiments, the patient is naïve to SGLT2 inhibitors, and has an eGFR of 20-90 mL/min/1.73 m² and a UACR of greater than 700 mg/g. In embodiments, the patient is naïve to SGLT2 inhibitors, and has an eGFR of 20-90 mL/min/1.73 m² and a UPCR of greater than 1.0 g/g. In embodiments, the patient is naïve to SGLT2 inhibitors, and has a UACR of greater than 700 mg/g and a UPCR of greater than 1.0 g/g. In embodiments, the patient is naïve to SGLT2 inhibitors, and has an eGFR of 20-90 mL/min/1.73 m², a UACR of greater than 700 mg/g, and a UPCR of greater than 1.0 g/g.

In embodiments, the patient is naïve to SGLT2 inhibitors, and has an eGFR of 20-90 mL/min/1.73 m² and a UACR of 700-900 mg/g. In embodiments, the patient is naïve to SGLT2 inhibitors, and has an eGFR of 20-90 mL/min/1.73 m² and a UPCR of 1.0-1.3 g/g. In embodiments, the patient is naïve to SGLT2 inhibitors, and has a UACR of 700-900 mg/g and a UPCR of 1.0-1.3 g/g. In embodiments, the patient is naïve to SGLT2 inhibitors and has an eGFR of 20-90 mL/min/1.73 m², a UACR of 700-900 mg/g, and a UPCR of 1.0-1.3 g/g.

In embodiments, the patient is not naive to SGLT2 inhibitors, and has an eGFR of 20-90 mL/min/1.73 m² and a UACR of greater than 700 mg/g. In embodiments, the patient is not naive to SGLT2 inhibitors, and has an eGFR of 20-90 mL/min/1.73 m² and a UPCR of greater than 1.0 g/g. In embodiments, the patient is not naive to SGLT2 inhibitors, and has a UACR of greater than 700 mg/g and a UPCR of greater than 1.0 g/g. In embodiments, the patient is not naive to SGLT2 inhibitors and has an eGFR of 20-90 mL/min/1.73 m², a UACR of greater than 700 mg/g, and a UPCR of greater than 1.0 g/g.

In embodiments, the patient is not naive to SGLT2 inhibitors, and has an eGFR of 20-90 mL/min/1.73 m² and a UACR of 700-900 mg/g. In embodiments, the patient is not naive to SGLT2 inhibitors, and has an eGFR of 20-90 mL/min/1.73 m² and a UPCR of 1.0-1.3 g/g. In embodiments, the patient is not naive to SGLT2 inhibitors, and has a UACR of 700-900 mg/g and a UPCR of 1.0-1.3 g/g. In embodiments, the patient is not naive to SGLT2 inhibitors and has an eGFR of 20-90 mL/min/1.73 m², a UACR of 700-900 mg/g, and a UPCR of 1.0-1.3 g/g.

In embodiments, the patients described herein are administered a fixed-dose combination of zibotentan, or a pharmaceutically acceptable salt thereof, and dapagliflozin, or a pharmaceutically acceptable salt thereof.

In embodiments, zibotentan, or a pharmaceutically acceptable salt thereof, is administered once daily.

In embodiments, the total daily dose of zibotentan, or the pharmaceutically acceptable salt thereof, administered is 0.25-1.5 mg. In embodiments, the total daily dose of zibotentan is 0.25 mg. In embodiments, the total daily dose of zibotentan is 0.5 mg. In embodiments, the total daily dose of zibotentan is 0.75 mg. In embodiments, the total daily dose of zibotentan is 1.0 mg. In embodiments, the total daily dose of zibotentan is 1.25 mg. In embodiments, the total daily dose of zibotentan is 1.5 mg.

In embodiments, zibotentan, or a pharmaceutically acceptable salt thereof, is in tablet form. In embodiments, zibotentan, or a pharmaceutically acceptable salt thereof, is administered in the form of a pharmaceutical composition comprising one or more pharmaceutically acceptable excipients. In some embodiments, the composition comprises one or more pharmaceutical diluents, one or more pharmaceutical disintegrants, or one or more pharmaceutical lubricants.

In embodiments, dapagliflozin, or a pharmaceutically acceptable salt thereof, is administered once daily.

In an embodiment, dapagliflozin is in the form of a pharmaceutically acceptable solvate, mixed solvate, or complex. In some aspects provided herein, dapagliflozin is in the form of a non-crystalline solid. In some aspects provided herein, dapagliflozin is in the form of a crystalline solid. In some aspects provided herein, dapagliflozin is in the form of a (S)-propylene glycol ((S)-PG) solvate which has the structure:

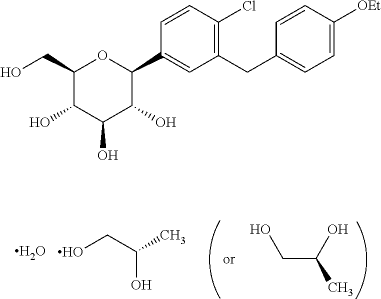

In aspects provided herein, dapagliflozin is administered to the patient orally. In aspects provided herein, dapagliflozin is administered to the patient in a tablet form.

In embodiments, the total daily dose of dapagliflozin, or the pharmaceutically acceptable salt thereof, administered is 2.5-10 mg. In embodiments, the total daily dose of dapagliflozin is 2.5 mg. In embodiments, the total daily dose of dapagliflozin is 3.0 mg. In embodiments, the total daily dose of dapagliflozin is 4.0 mg. In embodiments, the total daily dose of dapagliflozin is 5.0 mg. In embodiments, the total daily dose of dapagliflozin is 6.0 mg. In embodiments, the total daily dose of dapagliflozin is 7.0 mg. In embodiments, the total daily dose of dapagliflozin is 7.5 mg. In embodiments, the total daily dose of dapagliflozin is 8.0 mg. In embodiments, the total daily dose of dapagliflozin is 9.0 mg. In embodiments, the total daily dose of dapagliflozin is 10.0 mg.

In embodiments, zibotentan, or a pharmaceutically acceptable salt thereof, is administered once daily in a fixed-dose combination with dapagliflozin, or a pharmaceutically acceptable salt thereof.

In embodiments, a fixed-dose combination of zibotentan and dapagliflozin is administered comprising a total daily dose of 0.25-1.5 mg of zibotentan or a pharmaceutically acceptable salt thereof and a total daily dose of 2.5-10.0 mg of dapagliflozin or a pharmaceutically acceptable salt thereof. In embodiments, a fixed-dose combination of zibotentan and dapagliflozin is administered comprising a total daily dose of 0.25 mg of zibotentan or a pharmaceutically acceptable salt thereof and a total daily dose of 10.0 mg of dapagliflozin or a pharmaceutically acceptable salt thereof. In embodiments, a fixed-dose combination of zibotentan and dapagliflozin is administered comprising a total daily dose of 0.5 mg of zibotentan or a pharmaceutically acceptable salt thereof and a total daily dose of 10.0 mg of dapagliflozin or a pharmaceutically acceptable salt thereof. In embodiments, a fixed-dose combination of zibotentan and dapagliflozin is administered comprising a total daily dose of 0.75 mg of zibotentan or a pharmaceutically acceptable salt thereof and a total daily dose of 10.0 mg of dapagliflozin or a pharmaceutically acceptable salt thereof.

In embodiments, administration of the fixed-dose combination of zibotentan, or a pharmaceutically acceptable salt thereof, and dapagliflozin, or a pharmaceutically acceptable salt thereof to a patient in need thereof results in partial remission or remission of CKD. In embodiments, administration of the fixed-dose combination of zibotentan, or a pharmaceutically acceptable salt thereof, and dapagliflozin, or a pharmaceutically acceptable salt thereof to a patient in need thereof results in partial remission or remission of proteinuria. In embodiments, administration of the fixed-dose combination of zibotentan, or a pharmaceutically acceptable salt thereof, and dapagliflozin, or a pharmaceutically acceptable salt thereof to a patient in need thereof results in partial remission or remission of albuminuria. In embodiments, administration of the fixed-dose combination of zibotentan, or a pharmaceutically acceptable salt thereof, and dapagliflozin, or a pharmaceutically acceptable salt thereof to a patient in need thereof results in partial remission or remission of eGFR decline.

In embodiments, administration of the fixed-dose combination of zibotentan, or a pharmaceutically acceptable salt thereof, and dapagliflozin, or a pharmaceutically acceptable salt thereof to a patient in need thereof does not result in hospitalization of heart failure (hHF) for the patient. In embodiments, administration of the fixed-dose combination of zibotentan, or a pharmaceutically acceptable salt thereof, and dapagliflozin, or a pharmaceutically acceptable salt thereof to a patient in need thereof reduces the risk of hHF for the patient.

In embodiments, administration of the fixed-dose combination of zibotentan, or a pharmaceutically acceptable salt thereof, and dapagliflozin, or a pharmaceutically acceptable salt thereof to a patient in need thereof does not result in an increase in fluid retention in the patient. In embodiments, administration of the fixed-dose combination of zibotentan, or a pharmaceutically acceptable salt thereof, and dapagliflozin, or a pharmaceutically acceptable salt thereof to a patient in need thereof reduces the risk of increased fluid retention for the patient.

In embodiments, administration of the fixed-dose combination of zibotentan, or a pharmaceutically acceptable salt thereof, and dapagliflozin, or a pharmaceutically acceptable salt thereof to a patient in need thereof does not result in an increase in levels of brain natriuretic peptide (BNP) in the patient. In embodiments, administration of the fixed-dose combination of zibotentan, or a pharmaceutically acceptable salt thereof, and dapagliflozin, or a pharmaceutically acceptable salt thereof to a patient in need thereof reduces the risk of increased levels of BNP for the patient.

In embodiments, administration of the fixed-dose combination of zibotentan, or a pharmaceutically acceptable salt thereof, and dapagliflozin, or a pharmaceutically acceptable salt thereof to a patient in need thereof does not result in an increase in total body water in the patient. In embodiments, administration of the fixed-dose combination of zibotentan, or a pharmaceutically acceptable salt thereof, and dapagliflozin, or a pharmaceutically acceptable salt thereof to a patient in need thereof reduces the risk of increased total body water for the patient.

In embodiments, administration of the fixed-dose combination of zibotentan, or a pharmaceutically acceptable salt thereof, and dapagliflozin, or a pharmaceutically acceptable salt thereof to a patient in need thereof does not result in an increase in body weight in the patient. In embodiments, administration of the fixed-dose combination of zibotentan, or a pharmaceutically acceptable salt thereof, and dapagliflozin, or a pharmaceutically acceptable salt thereof to a patient in need thereof reduces the risk of increased body weight for the patient.

In embodiments, administration of the fixed-dose combination of zibotentan, or a pharmaceutically acceptable salt thereof, and dapagliflozin, or a pharmaceutically acceptable salt thereof to a patient in need thereof results in a reduction in the patient's blood pressure. In embodiments, administration of the fixed-dose combination of zibotentan, or a pharmaceutically acceptable salt thereof, and dapagliflozin, or a pharmaceutically acceptable salt thereof to a patient in need thereof reduces the risk of blood pressure elevation in the patient.

In embodiments, administration of the fixed-dose combination of zibotentan, or a pharmaceutically acceptable salt thereof, and dapagliflozin, or a pharmaceutically acceptable salt thereof to a patient in need thereof results in a reduction in the patient's cholesterol. In embodiments, administration of the fixed-dose combination of zibotentan, or a pharmaceutically acceptable salt thereof, and dapagliflozin, or a pharmaceutically acceptable salt thereof to a patient in need thereof reduces the risk of cholesterol elevation in the patient.

In embodiments, administration of the fixed-dose combination of zibotentan, or a pharmaceutically acceptable salt thereof, and dapagliflozin, or a pharmaceutically acceptable salt thereof to a patient in need thereof results in a reduction in the patient's hemoglobin A1c (HbA1c). In embodiments, administration of the fixed-dose combination of zibotentan, or a pharmaceutically acceptable salt thereof, and dapagliflozin, or a pharmaceutically acceptable salt thereof to a patient in need thereof reduces the risk of HbA1c elevation in the patient.

In embodiments, administration of the fixed-dose combination of zibotentan, or a pharmaceutically acceptable salt thereof, and dapagliflozin, or a pharmaceutically acceptable salt thereof to a patient having ANCA vasculitis and in need thereof results in a reduction of the incidence of ANCA vasculitis in the patient. In embodiments, administration of the fixed-dose combination of zibotentan, or a pharmaceutically acceptable salt thereof, and dapagliflozin, or a pharmaceutically acceptable salt thereof to a patient having ANCA vasculitis and in need thereof results in a reduction of the recurrence of ANCA vasculitis in the patient.

In embodiments, administration of the fixed-dose combination of zibotentan, or a pharmaceutically acceptable salt thereof, and dapagliflozin, or a pharmaceutically acceptable salt thereof to a patient having Alport syndrome and in need thereof results in a reduction of the progression of Alport syndrome in the patient. In embodiments, administration of the fixed-dose combination of zibotentan, or a pharmaceutically acceptable salt thereof, and dapagliflozin, or a pharmaceutically acceptable salt thereof to a patient having Alport syndrome and in need thereof results in a reduction of markers of kidney inflammation (nephritis) associated with Alport syndrome in the patient. In embodiments, administration of the fixed-dose combination of zibotentan, or a pharmaceutically acceptable salt thereof, and dapagliflozin, or a pharmaceutically acceptable salt thereof to a patient having Alport syndrome and in need thereof treats Alport syndrome in the patient.

In embodiments, administration of the fixed-dose combination of zibotentan, or a pharmaceutically acceptable salt thereof, and dapagliflozin, or a pharmaceutically acceptable salt thereof to a patient having biopsy-confirmed IgAN and in need thereof results in a reduction of proteinuria in the patient. In embodiments, administration of the fixed-dose combination of zibotentan, or a pharmaceutically acceptable salt thereof, and dapagliflozin, or a pharmaceutically acceptable salt thereof to a patient having biopsy-confirmed IgAN at risk of rapid disease progression and in need thereof results in a reduction of progression of kidney function decline associated with IgAN in the patient. In embodiments, administration of the fixed-dose combination of zibotentan, or a pharmaceutically acceptable salt thereof, and dapagliflozin, or a pharmaceutically acceptable salt thereof to a patient having biopsy-confirmed IgAN and in need thereof treats IgAN in the patient.

In embodiments, administration of the fixed-dose combination of zibotentan, or a pharmaceutically acceptable salt thereof, and dapagliflozin, or a pharmaceutically acceptable salt thereof to a patient in need thereof results in a reduction of the incidence of stroke in the patient. In embodiments, administration of the fixed-dose combination of zibotentan, or a pharmaceutically acceptable salt thereof, and dapagliflozin, or a pharmaceutically acceptable salt thereof to a patient in need thereof results in a reduction of the risk of stroke in the patient.

In some embodiments, the improvement in disease, reduction of incidence, reduction of risk, and other beneficial effects described herein and provided for through administration the fixed-dose combination of zibotentan and dapagliflozin as described in the preceding embodiments can represent improvements relative to the absence of therapy, improvements relative to placebo treatment, improvements relative to treatment with dapagliflozin alone, and/or improvements relative to treatment with other standard treatments for CKD and/or diseases, disorders, or conditions associated with high proteinuria chronic kidney disease.

In embodiments, the fixed-dose combination of zibotentan, or a pharmaceutically acceptable salt thereof, and dapagliflozin, or a pharmaceutically acceptable salt thereof, are administered concurrently. In embodiments, zibotentan, or a pharmaceutically acceptable salt thereof, is administered prior to administration of dapagliflozin, or a pharmaceutically acceptable salt thereof. In embodiments, zibotentan, or a pharmaceutically acceptable salt thereof, is administered after administration of dapagliflozin, or a pharmaceutically acceptable salt thereof.

Pharmaceutical compositions may be administered in any manner appropriate to the disease, disorder, and/or condition to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical composition can be formulated employing conventional solid or liquid vehicles, diluents, and pharmaceutical additives as appropriate for the mode of desired administration. The pharmaceutical compositions can be administered by a variety of routes including, for example, orally, in the form of tablets, capsules, granules, powders, and the like, parenterally, in the form of injectable preparations, intranasally, rectally, and transdermally, in the form of patches, for example.

The above dosage forms can also include a pharmaceutically acceptable carrier (i.e., a non-toxic, inert solid, semi-solid or liquid filler, diluent, encapsulating material or formulation auxiliary of any type), excipient, lubricant, buffer, antibacterial, bulking agent (such as mannitol), adjuvant, and the like.

Some examples of materials which can serve as pharmaceutically acceptable carriers are sugars, such as lactose, glucose and sucrose; starches, such as com starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such a propylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; and phosphate buffer solutions, as well as other non-toxic compatible lubricants, such as sodium lauryl sulfate and magnesium stearate; coloring agents; releasing agents; coating agents; sweetening; flavoring; and perfuming agents; preservatives; and antioxidants.

Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters, such as ethyl oleate. Proper fluidity can be maintained, for example, by the use of a coating such as lecithin, by the maintenance of the required particle size in the case of dispersions, and by the use of surfactants.

Examples of adjuvants include preservative agents, wetting agents, emulsifying agents, dispersing agents, suspending agents, sweetening, flavoring, and perfuming agents. Prevention of the action of microorganisms can be ensured by various antibacterial and antifungal agents, for example, parabens, chlorobutanol, phenol, sorbic acid, and the like. It can also be desirable to include isotonic agents, for example, sugars, sodium chloride and the like. Prolonged absorption of the injectable pharmaceutical form can be brought about by the use of agents delaying absorption, for example, aluminum monostearate and gelatin. Suspending agents include, for example, ethoxylated isostearyl alcohols, polyoxyethylene sorbitol, and sorbitan esters, microcrystalline cellulose, aluminum metahydroxide, bentonite, agar-agar, tragacanth, and mixtures thereof.

The various pharmaceutical compositions employed in the methods of the disclosure can optionally include one or more fillers or excipients in an amount within the range of from about 0% to about 90% by weight and in some embodiments from about 1% to about 80% by weight. Examples of suitable fillers or excipients include, but are not limited to, lactose, sugar, corn starch, modified corn starch, mannitol, sorbitol, inorganic salts, such as calcium carbonate, and cellulose derivatives, such as wood cellulose and microcrystalline cellulose.

One or more binders can be present in addition to or in lieu of the fillers in an amount within the range of from about 0% to about 35%. In some embodiments, the binders are present in an amount of from about 0.5% to about 30% by weight of the composition. Examples of suitable binders include polyvinylpyrrolidone (molecular weight ranging from about 5000 to about 80,000 and in some embodiments about 40,000), lactose, starches, such as corn starch, modified corn starch, sugars, gum acacia and the like, as well as a wax binder in finely powdered form (less than 500 microns), such as camauba wax, paraffin, spermaceti, polyethylenes and microcrystalline wax.

In some embodiments, the pharmaceutical composition is in the form of a tablet, wherein the tablet includes one or more tableting lubricants in an amount within the range of from about 0.2% to about 8% by weight of composition. In some embodiments, the tableting lubricant(s) is in an amount within the range of from about 0.5% to about 2% by weight of the composition. Examples of suitable tableting lubricants include, but are not limited to, magnesium stearate, stearic acid, palmitic acid, calcium stearate, talc, camauba wax, and the like. Other ingredients can optionally be present, including, for example, preservatives, stabilizers, colorants, anti-adherents and silica flow conditioners or glidants, such as Syloid brand silicon dioxide.

In some embodiments, the pharmaceutical composition is in the form of a tablet, wherein the tablet includes a coating layer which can comprise from about 0% to about 15% by weight of the tablet composition. The coating layer can comprise any conventional coating formulations that can include, for example, one or more film-formers or binders and/or one or more plasticizers. Examples of suitable film-formers or binders include, but are not limited to, hydrophilic polymers, such as hydroxypropylmethylcellulose, hydrophobic polymers, such as methacrylic acid esters, neutral polymers, ethyl cellulose, cellulose acetate, polyvinyl alcohol-maleic anhydride copolymers, β-pinene polymers, glyceryl esters of wood resins and the like. Examples of suitable plasticizers include, but are not limited to, triethyl citrate, diethyl phthalate, propylene glycol, glycerin, butyl phthalate, castor oil and the like. Both core tablets as well as coating formulations can contain aluminum lakes to provide color.

In some embodiments, the pharmaceutical composition is in the form of a tablet, wherein film-formers are applied to the tablet from a solvent system containing one or more solvents including water, alcohols such as methyl alcohol, ethyl alcohol and isopropyl alcohol, ketones such as acetone and ethylmethyl ketone, chlorinated hydrocarbons such as methylene chloride, dichloroethane, and 1,1,1-trichloroethane.

In some embodiments, the pharmaceutical composition is in the form of a tablet, wherein color is applied together with the film former, plasticizer, and solvent compositions.

In some embodiments, the pharmaceutical composition for use in the methods of the disclosure in the form of a tablet can be obtained by a process comprising the steps of:
a) mixing the inactive ingredients with the at least one compound of Formula (I);
b) formulating granules;
c) drying and/or screening the granules;
d) blending the granules; and
e) tableting the blend obtained in (d) into tablets.

In some embodiments, step a) of the process employs impact blending or milling and/or sizing equipment. In some embodiments, the granules in step b) of the process are formulated by dry granulation, wet granulation, or direct compression. In some embodiments, the granules are formulated by dry granulation. In some embodiments, the granules in step d) of the process are blended with a tableting aid or a lubricant and filler.

In some embodiments, the pharmaceutical composition in the form of a capsule can be obtained by a process comprising the steps of:
a) mixing the inactive ingredients with the at least one compound of Formula (I) using a combination of blending and milling processes;
b) formulating granules;
c) drying and/or screening the granules; and
d) loading the granules into capsules.

In some embodiments, step a) of the process employs impact milling or blending and/or sizing equipment. In some embodiments, the granules in step b) of the process are formulated by dry granulation, wet granulation, or direct compression. In some embodiments, the granules are formulated by dry granulation.

In some embodiments, the pharmaceutical composition may also contain adjuvants such as preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents which delay absorption such as aluminum monostearate and gelatin.

In some embodiments, it is desirable to slow the absorption of the drug from subcutaneous or intramuscular injection. In some embodiments, this is accomplished by the use of a liquid suspension of crystalline or amorphous material with poor water solubility. The rate of absorption of the drug then depends upon its rate of dissolution which, in turn, may depend upon crystal size and crystalline form. In some embodiments, delayed absorption is accomplished by dissolving or suspending the drug in an oil vehicle.

In some embodiments, the pharmaceutical composition is in an injectable depot form. In some embodiments, the injectable depot form comprises microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers suitable for use herein include poly(orthoesters) and poly(anhydrides). In some embodiments, depot injectable formulations are prepared by entrapping the drug in liposomes or microemulsions which are compatible with body tissues.

In some embodiments, the pharmaceutical composition is an injectable formulation, wherein the injectable formulation may be sterilized, for example, by filtration through a bacterial-retaining filter or by incorporating sterilizing agents in the form of sterile solid compositions which can be dissolved or dispersed in sterile water or other sterile injectable medium just prior to use.

In some embodiments, the pharmaceutical composition is a solid dosage form suitable for oral administration. Solid dosage forms for oral administration include capsules, tablets, pills, powders, and granules. In some embodiments, the at least one compound chosen from compounds of Formula (I) and prodrugs thereof is mixed with at least one inert, pharmaceutically acceptable excipient or carrier, such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quatemary ammonium compounds; g) wetting agents such as cetyl alcohol and glycerol monostearate; h) absorbents such as kaolin and bentonite clay, and/or i) lubricants such as talc, calcium stearate, magnesium stearate, solid polyethylene glycols, sodium lauryl sulfate, and mixtures thereof. In some embodiments, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

In some embodiments, tablets, dragees, capsules, pills, and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

In some embodiments the at least one compound chosen from compounds of Formula (I) and/or compounds of Formula (II) and/or prodrugs thereof may be in micro-encapsulated form, if appropriate, with one or more of the above-mentioned excipients.

In some embodiments the pharmaceutical composition may be in liquid dosage form suitable for oral administration including pharmaceutically acceptable emulsions, solutions, suspensions, syrups, and elixirs. In some embodiments, the liquid dosage form may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols, and fatty acid esters of sorbitan, and mixtures thereof.

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### EXAMPLES

### Example 1

Clinical trials of the fixed-dose combination of dapagliflozin and zibotentan are ongoing, including a randomized, double-blind, parallel-group, active controlled study to assess the efficacy of the fixed-dose combination of dapagliflozin and zibotentan compared to dapagliflozin monotherapy in patients with CKD and high proteinuria.

### Study Design

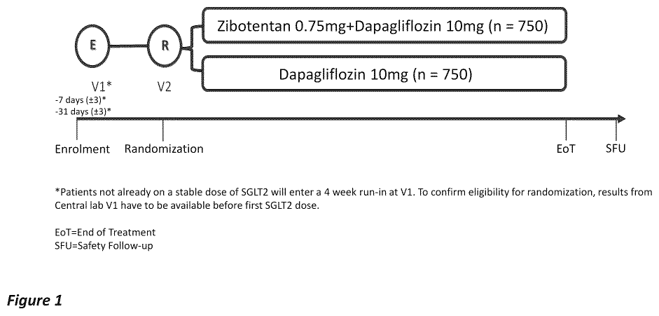

FIG. 1 illustrates the study timeline. Approximately 1500 patients ≥18 years of age with documented CKD (eGFR 220≥-<90 mL/min/1.73 m², with a UACR 2700-<900 mg/g or a UPCR≥1.0-<1.3 g/g and naïve to SGLT2 inhibitors) who meet eligibility criteria set forth in table 1 (inclusion criteria) and table 2 (exclusion criteria) will be randomized to the fixed-dose combination of zibotentan and dapagliflozin or dapagliflozin monotherapy in a 1:1 ratio as shown in table 3. Patients will be recruited for a period of approximately 18 months, followed by a 24-month treatment period and a 1-month safety follow-up for a total study length of approximately 43 months with an average follow-up period of approximately 33 months. All the variables will be collected to verify the inclusion criteria and additional demographics such as race/ethnicity, serum creatine, and height. A minimum of 30% of patients will have Type 2 diabetes, and 30s of patients without Type 2 diabetes will be randomized between the study groups.

TABLE 1
Inclusion criteria

| Inclusion Criteria | Rationale |
|---|---|
| Male and female aged 18 years at the | Safety |
| time of signing the informed consent. | |
| eGFR 20-< 90 mL/min/1.73 m². | The lower bound of eGFR is set as a limit |
| | where patients may progress but still |
| | have time for eGFR decline before an |
| | event of ESKD occurs. Upper bound for |
| | CKD is set with regards to benefit/risk |
| UACR >700-900¹ mg/g or UPCR ≥1-1.3¹ | High UACR and UPCR are predictors of |
| g/g. | more rapid progression on kidney |
| ¹Patients naïve to SGLT2 inhibitor | disease. High values for SGLT2 naïve |
| | subjects due to expected approximate |
| | 30% reduction after starting treatment |
| Provision of signed informed consent | GCP requirement |
| prior to any study-specific procedure. | |
| Receiving a dose of RAS inhibitor therapy | Background treatment is standard of |
| (ACEi or ARB) that has been stable for 12 | care, and this should be stable to avoid |
| weeks. Exceptions from this requirement | confounding |
| will be made for subjects who are unable | |
| to tolerate RAS inhibitor therapy. | |

TABLE 2
Exclusion criteria

| Exclusion Criteria | Rationale |
|---|---|
| New York Heart Association (NYHA) | Safety |
| class III or class IV Congestive Heart | |
| Failure at the time of enrollment. | |
| Minimal change disease (MCD) or rapidly | Not expected to demonstrate efficacy |
| progressive GN (RPGN). | |
| Participants with hospitalization for | Safety |
| unstable heart disease including HF | |
| within 3 months prior to screening. | |
| Participants with T1DM. | Safety |
| Prolonged QT interval (QTcF >470 ms) on | Safety |
| ECG at Randomization visit (Visit 2), | |
| known congenital long QT syndrome or | |
| history of QT prolongation associated | |
| with other medications. | |
| History of any life-threatening cardiac | Safety |
| dysrhythmia (continuous or paroxysmal). | |
| Child Pugh C or worse or unstable liver | Safety and potential PK effects |
| disease as exemplified by a bleed, | |
| worsening ascites or encephalopathy. | |
| Blood pressure above 150 mmHg systolic | BP treatment is SoC and should be |
| or 95 mmHg diastolic. | implemented first |
| Blood pressure below 90 mmHg systolic | Hypotension risk when treated with |
| or 60 mmHg diastolic. | zibotentan |
| Blood pressure below 90 mmHg systolic | Hypotension risk when treated with |
| or 60 mmHg diastolic. | zibotentan |
| Cardiac surgery or non-elective | Safety |
| percutaneous coronary interventions | |
| (PCI/TAVI) or open chest coronary artery | |
| bypass grafting [CABG] or valvular | |
| repair/replacement (within 3 months) prior | |
| to screening or is planned to undergo any | |
| of these procedures after randomization. | |
| History of solid organ transplantation. | Interpretation of data |
| Haemoglobin below 9 g/dL as measured | Risk for needing transfusion. If |
| by the Investigator, or blood transfusion | transfusions, risks of impacting lab values |
| for anaemia within the past 3 months. | |
| History or ongoing | Safety |
| allergy/hypersensitivity, as judged by the | |
| Investigator, to SGLT2i (e.g., | |
| dapagliflozin, canagliflozin, empagliflozin | |
| or other SGLT2-inhibitors) or Endothelin | |
| Receptor Antagonists (e.g., ambrisentan, | |
| atrasentan, bosentan, or other). | |
| Any condition outside the renal and CV | Avoiding confounding factors |
| disease area, with a life expectancy of | |
| less than 2 years based on investigator's | |
| clinical judgement, malignancy within the | |
| past 5 years. Exceptions to this criterion | |
| include non-melanoma skin cancer and | |
| curatively treated cervical carcinoma in | |
| situ. | |
| Participant hospitalized with heart | Safety, confounders |
| disease, such as MI, unstable angina, | |
| stroke, transient ischemic attack (TIA), | |
| carotid surgery, or carotid angioplasty or | |
| hospitalized due to COVID-19 infection | |
| during the last 3 months prior to | |
| screening. | |
| Severe hepatic impairment with aspartate | Safety interpretation |
| transaminase [AST] or alanine | |
| transaminase [ALT] >3× the upper limit of | |
| normal [ULN]; or total bilirubin >2× ULN | |
| at time of screening. An isolated increase | |
| in bilirubin in participants with known | |
| Gilbert's syndrome is not a reason for | |
| exclusion. | |
| Known blood-borne diseases (such as | Transport of blood samples as per IATA |
| Ebola, Lassa fever virus, Hepatitis A, B, | rules |
| C, D, and E viruses, and Human | |
| immunodeficiency virus (HIV) types 1 and | |
| 2). | |
| Inability of the patient, in the opinion of | Safety, interpretation |
| the Investigator, to understand and/or | |
| comply with IP, procedures and/or follow- | |
| up OR any conditions that, in the opinion | |
| of the Investigator, may render the patient | |
| unable to complete the study. | |
| Previous Randomization into the present | Safety, interpretation |
| study. | |
| Women of child-bearing potential | Including WOCBP for inclusiveness and |
| (WOCBP; i.e., those who are not | diversity, but minimising risk to fetus |
| chemically or surgically sterilised or who | |
| are not post-menopausal) who are not | |
| willing to use adequate contraception of | |
| at least 2 methods of birth control, of | |
| which at least one must be a highly | |
| effective birth control method¹ plus the | |
| use a condom by their male partner for | |
| sexual intercourse (e.g. condoms with | |
| hormonal contraception or implants or | |
| certain intrauterine devices), from the | |
| time of signing the informed consent | |
| throughout the study and 1 month | |
| thereafter, OR women who have a | |
| positive pregnancy test or a date of last | |
| menstruation not consistent with non- | |
| pregnancy at enrollment or | |
| Randomization. | |
| (Women are considered post- | |
| menopausal and not of child-bearing | |
| potential if they have had 12 months of | |
| natural (spontaneous) amenorrhea with | |
| an appropriate clinical profile (e.g., age | |
| appropriate) or 6 months of spontaneous | |
| amenorrhea with serum FSH levels | |
| >40 mlU/mL [for US only: FSH levels | |
| >40 mlU/mL and estradiol <20 pg/mL] or | |
| have had surgical treatment such as | |
| bilateral tubal ligation, | |
| bilateral ovariectomy/oophorectomy | |
| or hysterectomy.) | |
| ¹ Pending completion of oral contraceptive | |
| DDI study, if no significant interaction is | |
| demonstrated, oral contraceptives may | |
| become an acceptable method of birth | |
| control in a subsequent protocol amendment. | |
| Women who are pregnant, breast- | Known teratogenicity (zibotentan). |
| feeding, or women with intent of getting | Unknown risks with breast milk. |
| pregnant. | |
| Male participants who are not surgically | Unknown if potential teratogenic effect |
| sterile, abstinent, or in conjunction with a | from sperm/semen (safety) |
| female sexual partner who is | |
| of childbearing potential, are not using a | |
| highly effective method of contraception | |
| for the duration of the study (from the | |
| time they sign consent) and for 3 months | |
| after the last dose of investigational | |
| product to prevent any pregnancies. Male | |
| study participants must not donate or | |
| bank sperm during this same | |
| time period. | |
| Participation in another clinical study with | Safety, confounding |
| an investigational product administered in | |
| the last 3 months | |
| Clinically significant, unstable, or | Safety |
| uncontrolled medical condition as | |
| assessed by the Investigator | |
| Lupus nephritis, ANCA vasculitis, minimal | Interpretation of safety data or potential |
| change disease, ANCA vasculitis, | interactions with treatments |
| ADPKD (polycystic kidney disease), | |
| Alport syndrome, patients on RRT or | |
| previous kidney transplant | |
| Nephrotic syndrome (excluded if all 3 | Likely to require immunosuppression or to |
| present): | have complex AE profiles making |
| Proteinuria greater than 3-3.5 g/24 | interpretation of safety difficult |
| hour or spot UPCR of >300-350 | |
| mg/mmol | |
| Serum albumin <25 g/l | |
| Clinical evidence of peripheral oedema | |
| Patients on systemic immunosuppression | Interpretation of safety data |
| (inhaled, nasal or dermatological steroids | |
| are allowed) | |
| Known history of drug or alcohol abuse | Interpretation, safety |
| within 5 years prior to screening (Visit 1). | |

TABLE 3
Study groups for Example 1

| | | | Frequency | |
|---|---|---|---|---|
| Group | Test Article | Dose | and duration | Sample size |
| 1 | Dapagliflozin | 10 mg | QD, until | 750 |
| | | | study | |
| | | | completion | |
| 2 | Zibotentan | 0.75 mg | QD, until | 750 |
| | Dapagliflozin | 10 mg | study | |
| | | | completion | |

Patients will be administered either dapagliflozin (10 mg) monotherapy once daily, or the fixed-dose combination of zibotentan (0.75 mg) and dapagliflozin (10 mg) once daily. Patients not already on a stable dose of an SGLT2 inhibitor will receive a 4-week run-in period during which they will receive 10 mg of a SGLT2 inhibitor once daily prior to the start of the treatment period. Endpoints and objectives will be assessed in participants at site visits corresponding to the pre-randomization screening, randomization, and on day 14 (±3), 90 (±10), 180 (±10), 270 (±10), 360 (±10), 480 (±10), 600 (±10), and 720 (±7) of the study.

### Objectives and Endpoints

The primary endpoints will measure the effect of the fixed-dose combination of zibotentan and dapagliflozin versus dapagliflozin monotherapy on eGFR, UPCR, and UACR. The endpoints and objectives, including secondary endpoints and objectives are summarized in Table 4.

TABLE 4
Primary, Secondary, Exploratory, and Safety Endpoints and Objectives

| Objective/Endpoint | Description |
|---|---|
| To determine whether the fixed-dose | eGFR slope |
| combination of zibotentan and | |
| dapagliflozin is superior to dapagliflozin | |
| monotherapy to slow decline in renal | |
| function | |
| To determine whether the fixed-dose | Change from baseline in UPCR |
| combination of zibotentan and | |
| dapagliflozin is superior to dapagliflozin | |
| monotherapy in reducing proteinuria | |
| To determine whether the fixed-dose | Change from baseline in UACR |
| combination of zibotentan and | |
| dapagliflozin is superior to dapagliflozin | |
| monotherapy in reducing albuminuria | |
| To determine whether the fixed-dose | Time to the first occurrence of any of the |
| combination of zibotentan and | components of the composite endpoint of |
| dapagliflozin is superior to dapagliflozin | 30% sustained decline in eGFR or ESKD |
| monotherapy in reducing the incidence of | or renal death |
| the composite endpoint of 30% sustained | |
| decline in eGFR or ESKD or renal death | |
| To determine whether the fixed-dose | Time to the first occurrence of any of the |
| combination of zibotentan and | components of the composite endpoint of |
| dapagliflozin is superior to dapagliflozin | 40% sustained decline in eGFR or ESKD |
| monotherapy in reducing the incidence of | or renal death |
| the composite endpoint of 40% sustained | |
| decline in eGFR or ESKD or renal death | |
| To determine whether the fixed-dose | Time to the first occurrence of any of the |
| combination of zibotentan and | components of the composite endpoint of |
| dapagliflozin is superior to dapagliflozin | 57% sustained decline in eGFR or ESKD |
| monotherapy in reducing the incidence of | or renal death |
| the composite endpoint of 57% sustained | |
| decline in eGFR or ESKD or renal death | |
| To determine whether the fixed-dose | Proportion of patients achieving remission |
| combination of zibotentan and | and partial remission at end of study |
| dapagliflozin is superior to dapagliflozin | |
| monotherapy in increasing the proportion | |
| of patients with nephrotic range | |
| proteinuria who achieve partial remission | |
| or remission | |
| To determine whether the fixed-dose | Proportion of patients with UACR <300 |
| combination of zibotentan and | mg/g at end of study |
| dapagliflozin is superior to dapagliflozin | |
| monotherapy in increasing the proportion | |
| of patients achieving <300 mg/g UACR | |
| To determine the effect of the fixed-dose | Change from baseline in the overall |
| combination of zibotentan and | summary score of the EQ-5D at end of |
| dapagliflozin compared to dapagliflozin | study |
| monotherapy on the EQ-5D questionnaire | |
| (provided in Appendix A) | |
| To determine whether the fixed-dose | Time to the first occurrence of these |
| combination of zibotentan and | outcomes |
| dapagliflozin is superior to dapagliflozin | |
| monotherapy in reducing the incidence of | |
| stroke and acute coronary syndrome | |
| To characterize the plasma exposure of | Plasma concentration per treatment |
| zibotentan and dapagliflozin | group and study visit |
| To assess the safety and tolerability of | All adverse events (AEs) and Serious |
| treatment with zibotentan and | adverse events (SAEs) |
| dapagliflozin in fixed-dose combination | Discontinuation of investigational product |
| compared to dapagliflozin alone | (IP) due to adverse events (DAEs) |
| | Change in clinical chemistry/hematology |
| | parameters |
| | AEs of special interest (heart failure and |
| | fluid retention) |
| | ECG |
| | Vital signs |

### Appendix A (EQ-5D-5L Questionnaire)

### Health Questionnaire

Under each heading, please check the ONE box that best describes your health TODAY
We would like to know how good or bad your health is TODAY.
This scale is numbered from 0 to 100.
100 means the best health you can imagine.
0 means the worst health you can imagine.
Mark an X on the scale to indicate how your health is TODAY.
Now, please write the number you marked on the scale in the box below.

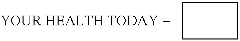

USA (English) © 2009 EuroQol Group. EQ-SD™ is a trade mark of the EuroQol Group

### Example 2

Clinical trials of the fixed-dose combination of dapagliflozin and zibotentan are ongoing, including a randomized, double-blind, parallel-group, active controlled study to assess the efficacy of fixed-dose combination (FDC) of dapagliflozin and zibotentan compared to dapagliflozin monotherapy in patients with CKD and high proteinuria (see Example 1). Example 2 provides a modified protocol comprising a dose-titration for zibotentan in the fixed-dose combination.

### Study Design

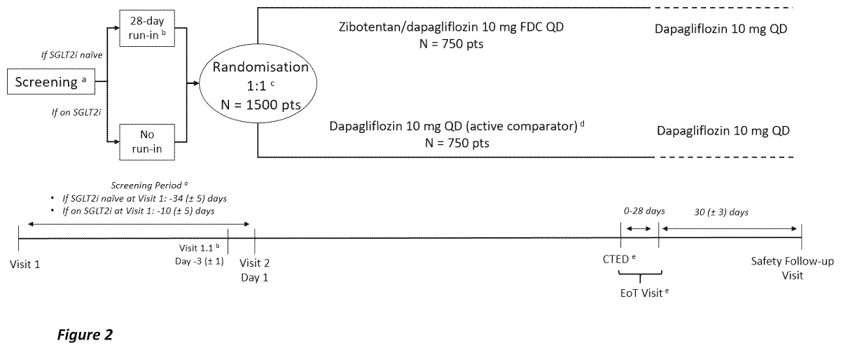

FIG. 2 illustrates the study timeline. Approximately 1500 patients who meet eligibility criteria (see inclusion and exclusion criteria in Tables 5A and 5B, respectively) will be randomized into one of two treatment arms in a 1:1 ratio:
Zibotentan/Dapagliflozin in a FDC orally administered once daily.
Dapagliflozin 10 mg orally administered once daily (active comparator).

TABLE 5A
Study Inclusion Criteria.
Inclusion Criteria

Age
≥18 years of age.
Type of Participant and Disease Characteristics/Laboratory Parameters
Diagnosis of CKD, defined as:
1. eGFR ≥20 and <90 mL/min/1.73 m² by central laboratory at screening
Visit 1, using CKD-EPI formula (Error! Reference source not found.).
AND
2. UACR >700 mg/g (>79 mg/mmol) or UPCR >1000 mg/g
(>113 mg/mmoL).
This value must be from one of the following:
1. A local laboratory value obtained within 4 months prior to screening
Visit 1, and the participant must be in a usual state of health without any
modifications to RAASi (ACEi or ARB), MRA, or SGLT2i therapy.
Note: Protein excretion rate (PER) ≥1000 mg/day may be used for
inclusion (instead of UPCR) if PER is used locally for quantification.
2. A central laboratory value obtained at screening Visit 1.
Reproduction
Female participants must have a negative serum pregnancy test result at
screening
Female participants must be either:
1. Not of child-bearing potential: to be confirmed at screening by
fulfilling one of the following criteria:
(i) postmenopausal defined as amenorrhea for at least 12 months or
more following cessation of all exogenous hormonal treatments and
follicle-stimulating hormone (FSH) levels in the postmenopausal
range. In the absence of 12 months of amenorrhea, a single FSH
measurement is insufficient to define postmenopausal criteria. In
case of perimenopause or infrequent periods with variable levels of
FSH, women should be considered of childbearing potential.
(ii) documentation of irreversible surgical sterilization by hysterectomy,
bilateral oophorectomy, or bilateral salpingectomy but not tubal
ligation.
OR
2. Women of child bearing potential (WOCBP) using at least one
highly effective birth control method¹ (defined as one that can achieve a
failure rate of less than 1% per year when used consistently and
correctly) for at least 3 months prior to first dose of study intervention.
In addition, throughout the study until at least 1 month after the last
dose of study intervention, WOCBP must use adequate contraception
of at least 2 methods of birth control; the 2 birth control methods must
include (1) a highly effective birth control method PLUS (2) the use
of a condom by their male partner for sexual intercourse.
Other Inclusion Criteria
Receiving RAASi therapy (ACEi or ARB), and for the patient maximum
tolerated labelled daily dose, that has been stable for at least 4 weeks.
NOTE: If local laboratory is used per the inclusion criteria, the RAASi
therapy (ACEi or ARB), MRA, or SGLT2i inhibitor must have been
stable at least 4 weeks and from the time when the local laboratory
value was obtained. Exceptions from this requirement will be made for
participants who are unable to tolerate RAASi therapy.
¹Highly effective birth control methods include:
Total sexual abstinence, provided it is the usual lifestyle of the participant (defined as refraining from heterosexual intercourse during the entire period of risk associated with the study treatments). Note that periodic abstinence (e.g., calendar, ovulation, symptothermal, post-ovulation methods), declaration of abstinence for the duration of exposure to study intervention, and withdrawal are not acceptable methods of contraception.
A vasectomised partner, Implanon ®, bilateral tubal occlusion, intrauterine device/levonorgestrel intrauterine system, Depo-Provera ™ injections, oral contraceptives containing ethinyl estradiol and levonorgestrel, Evra Patch ™, Xulane ™, or NuvaRing ®.

TABLE 5B
Study Exclusion Criteria.
Exclusion Criteria

Medical Conditions
Participants with NYHA class III or class IV Congestive HF at the time of
enrolment.
Participants with T1DM.
History of any life-threatening ventricular dysrhythmia (continuous or
paroxysmal).
Blood pressure above 160 mmHg systolic.
Blood pressure below 90 mmHg systolic or 60 mmHg diastolic.
Participants hospitalized for heart disease (heart failure, myocardial
infarction, angina, cerebrovascular accident) or cardiac procedures
(carotid surgery/plasty, CABG/PCI/TAVI, or valve replacement) or
for COVID-19 during the last 3 months prior to screening.
History of solid organ transplantation or bone marrow transplant.
History or ongoing allergy/hypersensitivity, as judged by the
Investigator, to SGLT2i therapy (e.g., dapagliflozin, canagliflozin,
empagliflozin or other SGLT2 inhibitors) or Endothelin Receptor
Antagonists (e.g., ambrisentan, atrasentan, bosentan, or other).
Any condition with a life expectancy of less than 2 years based on
investigator's clinical judgment, malignancy within the past 5 years.
Exceptions to this criterion include non-melanoma skin cancer and
curatively treated cervical carcinoma in situ.
Significant liver disease as judged by the investigator or severe hepatic
impairment with AST or ALT >3× ULN; or total bilirubin >2× ULN at
time of screening. An isolated increase in bilirubin in participants with
known Gilbert's syndrome is not a reason for exclusion.
Known blood-borne diseases such as specified in Error! Reference
source not found. (category A and B).
Clinically significant, unstable, or uncontrolled medical condition as
assessed by the Investigator.
Participants on renal replacement therapy or previous kidney transplant.
Known history of drug or alcohol abuse within 12 months of screening
(Visit 1).
Prior/Concurrent Therapy
Participants on treatment with strong or moderate CYP3A4 inducer.
Participants on systemic immunosuppression therapy other than stable
maintenance therapy defined as prednisone 10 mg/day (or equivalent) or
less; aziothioprine 100 mg/day or less; MMF 1000 mg/day or less
for at least 3 months prior to Visit 1. Inhaled, nasal or dermatological
steroids are also allowed.
Participants treated or expecting to be treated with tolvaptan, any other
ERAs, or budesonide (where used to treat IBD or IgAN).
Prior/Concurrent Clinical Study Experience
Participation in another clinical study with a study intervention
administered in the last 3 months.
Other Exclusions
Involvement in the planning and/or conduct of the study (applies to both
AstraZeneca staff and/or staff at the study site).
Inability of the participant, in the opinion of the investigator, to
understand and/or comply with investigational medicinal product (IMP),
procedures and/or follow-up OR any conditions that, in the opinion of
the Investigator, may render the participant unable to complete the study.
Previous randomization in the present study.
Women who are pregnant, breast-feeding, or women with intent of
getting pregnant.
Women who are not willing to use adequate contraception or cannot, in
the opinion of the investigator, understand and/or comply with the
study procedures.

Throughout the study until at least 1 month after the last dose of study intervention, women of child bearing potential (WOCBP) will use adequate contraception of at least 2 methods of birth control; the 2 birth control methods will include (1) a highly effective birth control method (see inclusion criterion 4 for definition and list of highly effective methods of contraception) PLUS (2) the use of a condom by their male partner for sexual intercourse. Pregnancy testing and contraception adherence checks will be performed.

A screen failure occurs when a participant has consented to participate in the clinical study but is not subsequently randomized. A minimal set of screen failure information is required to ensure transparent reporting of screen failure participants to meet the Consolidated Standards of Reporting Trials (CONSORT) publishing requirements and to respond to queries from regulatory authorities. Minimal information includes demography, screen failure details, eligibility criteria, and any serious adverse event (SAE).

Individuals who do not meet the criteria for participation in this study (screen failure) may be fully re-screened once. Re-screened participants will re-sign informed consent and be assigned the same participant number as for the initial screening.

Where the values for the following investigations are outside the usual range for a participant during screening, based on their medical history, re-testing will be undertaken on 2 occasions without requiring a re-screen: blood pressure, eGFR, spot urine for ACR, ALT, AST, and bilirubin.

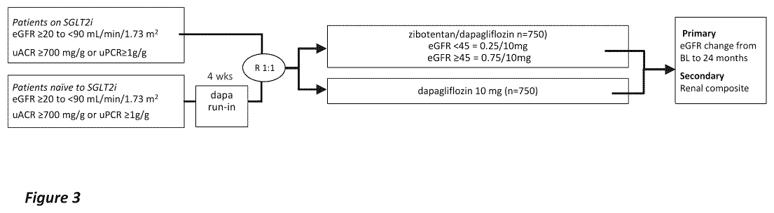

Study participants who are not already on SGLT2i at the initial screening visit (visit 1) will receive a 28-day run-in intervention with SGLT2i administered once daily. All participants, including those already on a stable dose of SGLT2i at the initial screening visit (visit 1), will take their last dose of the prescribed SGLT2i one day prior to randomization (day −1) and will switch to the study intervention according to their randomized treatment arm assignment on visit 2 (day 1). The dosage of the zibotentan/dapagliflozin fixed-dose combination or dapagliflozin monotherapy is determined according to eGFR status at the initial screening visit (visit 1), as specified below and in FIG. 3:
eGFR<45 mL/min/1.73 m²: Randomized 1:1 to zibotentan/dapagliflozin 0.25 mg/10 mg FDC QD or dapagliflozin 10 mg QD (active comparator)
eGFR≥45 mL/min/1.73 m²: Randomized 1:1 to zibotentan/dapagliflozin 0.75 mg/10 mg FDC QD or dapagliflozin 10 mg QD (active comparator)

Capping will be utilized to ensure adequate representation with regard to participants with or without Type 2 diabetes mellitus (T2D) and by eGFR status at screening (eGFR< or ≥45 mL/min/1.73 m²).

To ensure blinding to treatment, the zibotentan/dapagliflozin 0.25 mg/10 mg FDC, zibotentan/dapagliflozin 0.75 mg/10 mg FDC, and dapagliflozin 10 mg tablets will be identical in appearance (matching and indistinguishable from the zibotentan/dapagliflozin FDC). Participants, site personnel, and the Sponsor will all be blinded to the study intervention. Information on the study intervention is provided below in Table 6.

TABLE 6
Study Interventions.

| Arm name | Zibotentan/dapagliflozin ^{a, b} | | Dapagliflozin |
|---|---|---|---|
| Intervention | Zibotentan/ | Zibotentan/ | Dapagliflozin 10 mg |
| name | dapagliflozin | dapagliflozin | |
| | 0.25 mg/10 mg | 0.75 mg/10 mg | |
| Type | Drug | Drug | Drug |
| Dose | Film-coated | Film-coated | Film-coated tablet |
| formulation | tablet | tablet | (matching |
| | | | zibotentan/ |
| | | | dapagliflozin |
| | | | tablet) |
| Unit dose | 0.25 mg/10 mg | 0.75 mg/10 mg | 10 mg |
| strength(s) | | | |
| Dosage | 1 tablet of | 1 tablet of | 1 tablet of |
| level(s) | zibotentan/ | zibotentan/ | dapagliflozin |
| | dapagliflozin | dapagliflozin | 10 mg QD |
| | 0.25 mg/10 mg FDC | 0.75 mg/10 mg FDC | |
| | QD | QD | |
| Route of | Oral | Oral | Oral |
| administration | | | |
| Use | Experimental | Experimental | Active comparator |
| IMP or NIMP | IMP | IMP | IMP |

| | |
|---|---|
| Sourcing | Zibotentan/dapagliflozin and dapagliflozin treatments |
| | will be supplied centrally through AstraZeneca |
| Packaging | Zibotentan/dapagliflozin and dapagliflozin treatments will |
| and labelling | be provided in matching bottles. Each bottle will be labelled |
| | in accordance with country regulatory requirement. |

^{a} Zibotentan/dapagliflozin dose will be based on eGFR status at screening (Visit 1): 0.25 mg/10 mg FDC if eGFR <45 mL/min/1.73 m² and 0.75 mg/10 mg FDC if eGFR ≥45 mL/min/1.73 m².
^{b} Dose modifications are made as described herein.
FDC = fixed-dose combination;
IMP = investigational medicinal product;
NIMP = non-investigational medicinal product;
QD = once daily.

Participants will receive blinded study intervention from Day 1 until the end of treatment (EoT) visit. The common treatment end date (CTED), defined as 2 years after the date of randomization of the last participant in the study, will be used to determine when the EoT visit should occur. The EoT visit will occur preferably within 14 days and not more than 28 days after the CTED for all participants. At the EoT visit, participants will stop the blinded study intervention and will start open-label dapagliflozin 10 mg monotherapy until the safety follow-up visit (last study visit).

In the event of premature discontinuation of the blinded study intervention, participants will continue in the study and receive open label dapagliflozin 10 mg monotherapy, unless, in the opinion of the investigator, the participant is not able to tolerate dapagliflozin. The EoT visit will be the last study visit for these participants.

All participants will be followed in the study until 24 months after the last participant is randomized. The participant recruitment period is 18 months, and the study length will therefore be approximately 43 months, with the average follow-up period being approximately 33 months.

Participants will have 4 visits during the first 3 months of treatment (Days 1, 14, 30, and 90), after which visits will occur every 3 months during the first year (through Day 360) and every 4 months thereafter. One additional visit will be required one week after Visit 11 (Day 720). When the CTED is determined, the EoT visit will be scheduled to occur within 28 days after the CTED. A safety follow-up visit will occur one month after the EoT visit.

The schedules of study activities are outlined in Tables 7A, 7B, and 7C, for Screening & Randomization of SGLT2i-naïve participants (Table 7A), Screening & Randomization of SGLT2i-treated participants (Table 7B), and On-treatment, Study Intervention Discontinuation, and Safety Follow-up (Table 7C).

TABLE 7A
Schedule of Activities: Screening and Randomization for SGLT2 Inhibitor-Naïve Participants.

| | Procedure | | |
|---|---|---|---|
| | Screening ^{a} | | Randomization |
| | Visit | | |
| | 1 | 1.1 | 2 |
| | Day | | |
| | −34 (±5) ^{b} | −3 (±1) | 1 |
| | Month | | |
| | −1 | −1 | 0 |
| Informed consent | X | | |
| Enrolment in IRT/RTSM | X | | |
| Demography | X | | |
| Inclusion and exclusion criteria | X | | |
| Medical/ surgical history (includes substance usage [and family | X | | |
| history of premature CV disease]) | | | |
| Serum pregnancy test ^{c} | X | | |
| Urine pregnancy test and contraception adherence check | | | X |
| (WOCBP only) ^{d, e} | | | |
| FSH testing ^{e} | X | | |
| Randomization in IRT/RTSM | | | X |
| Height | X | | |
| Vital signs (BP, pulse and body weight) | X | | X |
| Full physical examination | X | | |
| Targeted physical examination ^{f} | | | X |
| NYHA Functional Classification | X | | |
| 12-lead ECG | | | X |
| Concomitant medication | X | | X |
| Serology (HIV I and II, hepatitis B surface antigen, hepatitis C | X | | |
| virus antibody, central laboratory) | | | |
| Random spot urine for UACR and UPCR: U-albumin, U-protein | X | X | X |
| and U-creatinine (central laboratory) ^{a} | | | |
| Clinical chemistry and hematology (central laboratory) ^{a, g} | X | | X |
| Additional blood sample for creatinine (eGFR calculation) | | X | |
| AEs including SAEs, DAEs, SAEs with outcome of death, | X (SAEs | X (SAEs | X |
| AESIs | only) | only) | |
| EQ-5D-5L (including EQ-VAS) | | | X |
| SPFQ | | | X |
| Study intervention intake at the clinic | | | X |
| Dispense study intervention | | | X |
| Optional samples for future biomarker research (plasma, serum | | | X |
| and urine) ^{h} | | | |
| Genomics Initiative optional, exploratory genomics, | | | X |
| transcriptomic, and multi-omics samples ⁱ | | | |

Note:
PRO questionnaires should be completed prior to any other study procedures or discussions (following informed consent), including medication treatments, to avoid biasing the participant's responses to the questions.
^{a} Individuals who do not meet the criteria for participation in this study (screen failure) may be fully re-screened once. Re-screened participants should re-sign informed consent and be assigned the same participant number as for the initial screening. Where the values for the following investigations are outside the usual range for a participant during screening, based on their medical history, re-testing may be undertaken on up to two occasions without requiring a re-screen: blood pressure, eGFR, spot urine for UACR and UPCR, ALT, AST, and bilirubin.
^{b} The 10-day window (−34 [±5] days) for Visit 1 is to allow for turn-around time for central laboratory results before the 28-day run-in. Participants not already on a stable dose of SGLT2i will enter a 28-day run-in with SGLT2i from Day −28 through Day −1. During the run-in period, SGLT2i will be provided as an auxiliary drug.
^{c} Negative pregnancy test (serum) required for all women at the time of enrolment/prior to first dose of study intervention.
^{d} Monthly pregnancy test (urine) and contraception adherence check required for all WOCBP from randomization until 1 month after last dose. If positive, the treatment should be discontinued and procedures to be followed in the event of suspected contraceptive failure or suspicion of pregnancy. The company may consider options for pregnancy testing at home by a health care professional for months where no visit is scheduled.
^{e} Women not of childbearing potential are to be confirmed at screening by fulfilling one of the following criteria: (a) postmenopausal defined as amenorrhea for at least 12 months or more following cessation of all exogenous hormonal treatments and FSH levels in the postmenopausal range. In the absence of 12 months of amenorrhea, a single FSH measurement is insufficient to define postmenopausal criteria. In case of perimenopause or infrequent periods with variable levels of FSH, women should be considered of childbearing potential; or (b) documentation of irreversible surgical sterilization by hysterectomy, bilateral oophorectomy, or bilateral salpingectomy but not tubal ligation.
^{f} Including signs of fluid retention.
^{g} Tests to include: B-hemoglobin, B-leukocyte differential count (absolute count), B-platelet count, B-HbA1c, B-hematocrit, B-leukocyte count, B-BNP, B-red blood cell count, S-bicarbonate, S-chloride, S-GGT, S-glucose, S-albumin, S-ALP, S-ALT, S-AST, total S-bilirubin, S-BUN, total S-calcium, S-creatine kinase, S-creatinine (including eGFR assessment), S-magnesium, S-phosphorous, S-potassium, S-sodium.
^{h} Plasma, serum, and urine samples for potential future biomarker research are optional.
ⁱ Participation in Genomics Initiative Research is optional and subject to separate consent by the participant.
AE = adverse event; AESI = adverse event of special interest; ALP = alkaline phosphatase; ALT = alanine transaminase AST = aspartate aminotransferase; B = blood; BNP = B-type natriuretic peptide; BP = blood pressure; CV = cardiovascular; DAE = adverse event leading to discontinuation of investigational medicinal product; eGFR = estimated glomerular filtration rate; ECG = electrocardiogram; EQ-5D-5L = European Quality of Life 5-dimensional 5-level questionnaire; EQ-VAS = VAS = European Quality of Life Visual Analogue Scale; FSH = follicle stimulating hormone; GGT = gamma-glutamyl transferase; HbA1c = glycated hemoglobin; HIV = human immunodeficiency virus; IRT = Interactive Response Technology; NYHA = New York Heart Association; PRO = patient-reported outcome; PTDV = premature treatment discontinuation visit; Q4M = every 4 months; RTSM = Randomization and Trial Supply Management; S = serum; SAE = serious adverse event; S-BUN = S-blood urea nitrogen; SGLT2i = sodium-glucose co-transporter 2 inhibitor; SPFQ = Study Participant Feedback Questionnaire; UACR = urine albumin to creatinine ratio; UPCR = urine protein to creatinine ratio; WOCBP = women of childbearing potential.

TABLE 7B
Schedule of Activities: Screening and Randomization for SGLT2 Inhibitor-Treated Participants.

| | Procedure | |
|---|---|---|
| | Screening ^{a, b} | Randomization |
| | Visit | |
| | 1 | 2 |
| | Day | |
| | −10 (±5) | 1 |
| | Month | |
| | −1 | 0 |
| Informed consent | X | |
| Enrolment in IRT/RTSM | X | |
| Demography | X | |
| Inclusion and exclusion criteria | X | |
| Medical/surgical history (includes substance usage [and family history of | X | |
| premature CV disease]) | | |
| Serum pregnancy test ^{c} | X | |
| Urine pregnancy test and contraception adherence check (WOCBP | | X |
| only) ^{d, e} | | |
| FSH testing ^{e} | X | |
| Randomization in IRT/RTSM | | X |
| Height | X | |
| Vital signs (BP, pulse and body weight) | X | X |
| Full physical examination | X | |
| Targeted physical examination ^{f} | | X |
| NYHA Functional Classification | X | |
| 12-lead ECG | | X |
| Concomitant medication | X | X |
| Serology (HIV I and II, hepatitis B surface antigen, hepatitis C virus | X | |
| antibody, central laboratory) | | |
| Random spot urine for UACR and UPCR: U-albumin, U-protein and | X | X |
| U-creatinine (central laboratory) | | |
| Clinical chemistry and hematology (central laboratory) ^{g} | X | X |
| AEs including SAEs, DAEs, SAEs with outcome of death, AESIs | X (SAEs | X |
| | only) | |
| EQ-5D-5L (including EQ-VAS) | | X |
| SPFQ | | X |
| Study intervention intake at clinic | | X |
| Dispense study intervention | | X |
| Optional samples for future biomarker research (plasma, serum | | X |
| and urine) ^{h} | | |
| Genomics Initiative optional, exploratory genomics, transcriptomic, and | | X |
| multi-omics samples ⁱ | | |

Note:
PRO questionnaires should be completed prior to any other study procedures or discussions (following informed consent), including medication treatments, to avoid biasing the participant's responses to the questions.
^{a} Individuals who do not meet the criteria for participation in this study (screen failure) may be fully re-screened once. Re-screened participants should re-sign informed consent and be assigned the same participant number as for the initial screening. Where the values for the following investigations are outside the usual range for a participant during screening, based on their medical history, re-testing may be undertaken on up to two occasions without requiring a re-screen: blood pressure, eGFR, spot urine for UACR and UPCR, ALT, AST, and bilirubin.
^{b} Participants already on a stable dose of SGLT2i.
^{c} Negative pregnancy test (serum) required for all women at the time of enrolment/prior to first dose of study intervention.
^{d} Monthly pregnancy test (urine) and contraception adherence check required for all WOCBP from randomization until 1 month after last dose. If positive, the treatment should be discontinued and procedures to be followed in the event of suspected contraceptive failure or suspicion of pregnancy. The company may consider options for pregnancy testing at home by a health care professional for months where no visit is scheduled.
^{e} Women not of childbearing potential are to be confirmed at screening by fulfilling one of the following criteria: (a) postmenopausal defined as amenorrhea for at least 12 months or more following cessation of all exogenous hormonal treatments and FSH levels in the postmenopausal range. In the absence of 12 months of amenorrhea, a single FSH measurement is insufficient to define postmenopausal criteria. In case of perimenopause or infrequent periods with variable levels of FSH, women should be considered of childbearing potential; or (b) documentation of irreversible surgical sterilization by hysterectomy, bilateral oophorectomy, or bilateral salpingectomy but not tubal ligation.
^{f} Including signs of fluid retention.
^{g} Tests to include: B-hemoglobin, B-leukocyte differential count (absolute count), B-platelet count, B-HbA1c, B-hematocrit, B-leukocyte count, B-BNP, B-red blood cell count, S-bicarbonate, S-chloride, S-GGT, S-glucose, S-albumin, S-ALP, S-ALT, S-AST, total S-bilirubin, S-BUN, total S-calcium, S-creatine kinase, S-creatinine (including eGFR assessment), S-magnesium, S-phosphorous, S-potassium, S-sodium.
^{h} Plasma, serum, and urine samples for potential future biomarker research are optional.
ⁱ Participation in Genomics Initiative Research is optional and subject to separate consent by the participant.
AE = adverse event; AESI = adverse event of special interest; ALP = alkaline phosphatase; ALT = alanine transaminase AST = aspartate aminotransferase; B = blood; BNP = B-type natriuretic peptide; BP = blood pressure; CV = cardiovascular; DAE = adverse event leading to discontinuation of investigational medicinal product; eGFR = estimated glomerular filtration rate; ECG = electrocardiogram; EQ-5D-5L = European Quality of Life 5-dimensional 5-level questionnaire; EQ-VAS = VAS = European Quality of Life Visual Analogue Scale; FSH = follicle stimulating hormone; GGT = gamma-glutamyl transferase; HbA1c = glycated hemoglobin; HIV = human immunodeficiency virus; IRT = Interactive Response Technology; NYHA = New York Heart Association; PRO = patient-reported outcome; PTDV = premature treatment discontinuation visit; Q4M = every 4 months; RTSM = Randomization and Trial Supply Management; S = serum; SAE = serious adverse event; S-BUN = S-blood urea nitrogen; SGLT2i = sodium-glucose co-transporter 2 inhibitor; SPFQ = Study Participant Feedback Questionnaire; UACR = urine albumin to creatinine ratio; UPCR = urine protein to creatinine ratio; WOCBP = women of childbearing potential.

TABLE 7C
Schedule of Activities: On-treatment, Study Intervention and Safety Follow-up.

| | Procedure | | | | | | | | | | |
|---|---|---|---|---|---|---|---|---|---|---|---|
| | Intervention Period | | | | | | | | | | |
| | Visit | | | | | | | | | | Safety |
| | 3 | 4 ^{b} | 5 | 6 | 7 | 8 | 9, 10, 11 etc. | 11.1 | PTDV | EoT ^{a} | Follow-up |
| | Day | | | | | | | | | | |
| | | | | | | | 480 (±10), | | | | |
| | | | | | | | 600 (±10), | | Within | | |
| | | | | | | | 720 (±7), | 7 (±3) | 28 days | Within | 30 (±3) |
| | 14 | 30 | 90 | 180 | 270 | 360 | then Q4M | days after | after | 28 days | days after |
| | (±3) | (±3) | (±10) | (±10) | (±10) | (±10) | (±7 days) | Visit 11 | last dose | after CTED ^{a} | EoT visit |
| | Month | | | | | | | | | | |
| | 0.5 | 1 | 3 | 6 | 9 | 12 | 16, 20, 24, etc | | | | |
| Urine pregnancy test and | | X (monthly) | | | | | | | X | X | X |
| contraception adherence | | | | | | | | | | | |
| check (WOCBP only) ^{c, d} | | | | | | | | | | | |
| Vital signs (BP, pulse | X | | X | X | X | X | X | | X | X | X |
| and body weight) | | | | | | | | | | | |
| Full physical examination | | | | | | | | | X | X | |
| Targeted physical | X | | X | X | X | X | X | | X | X | |
| examination ^{e} | | | | | | | | | | | |
| 12-lead ECG | | | | X | | X | X (yearly) | | X | X | |
| Concomitant medication | X | | X | X | X | X | X | | X | X | X |
| Random spot urine for UACR | X | | X | X | X | X | X | | X | X | X |
| and UPCR: U-albumin, U-protein | | | | | | | | | | | |
| and U-creatinine (central | | | | | | | | | | | |
| laboratory) | | | | | | | | | | | |
| Clinical chemistry and | X | | X | X | X | X | X | | X | X | X |
| hematology (central laboratory) ^{f} | | | | | | | | | | | |
| Additional blood sample for | | | | | | | | X ^{g} | | | |
| creatinine (eGFR calculation) | | | | | | | | | | | |
| PK sampling (pre-dose) ^{h} | X | | | X | | | X (Visit | | | | |
| | | | | | | | 9 only) | | | | |
| AEs including SAEs, DAEs, SAEs | X | X | X | X | X | X | X | X | X | X | X |
| with outcome of death, AESIs | | | | | | | | | | | |
| EQ-5D-5L (including EQ-VAS) | | | | X | | X | X (Visit | | X | X | |
| | | | | | | | 11 only) | | | | |
| SPFQ | | | | | | | X (Visit | | X | X | |
| | | | | | | | 11 only) | | | | |
| Dispense study intervention | X | | X | X | X | X | X | | | | |
| Accountability (study | X | | X | X | X | X | X | | X | X | |
| intervention compliance | | | | | | | | | | | |
| reminder, collect unused | | | | | | | | | | | |
| study intervention) | | | | | | | | | | | |
| Study intervention intake | X | | | X | | | X (Visit | | | | |
| at clinic ⁱ | | | | | | | 9 only) | | | | |
| Optional samples for future | | | | | | X | X (Visit | | | | |
| biomarker research (plasma, | | | | | | | 11 only) | | | | |
| serum and urine) ^{j} | | | | | | | | | | | |

Note:
PRO questionnaires should be completed prior to any other study procedures or discussions (following informed consent), including medication treatments, to avoid biasing the participant's responses to the questions.
^{a} The CTED is defined as 2 years after the date of randomization of the last participant in the study and will be used to determine when to schedule EoT visits. The EoT visit should occur preferably within 14 days and not more than 28 days after the CTED for all participants, including participants who have prematurely discontinued blinded study intervention. At the EoT visit, participants still on blinded treatment will discontinue blinded study intervention and start open-label monotherapy with dapagliflozin 10 mg QD until the safety follow-up visit. For participants who have prematurely discontinued study intervention but remained in the study as per the SoA, the EoT visit will be the final study visit.
^{b} Visit 4: phone call to assess fluid retention and related conditions.
^{c} Monthly pregnancy test (urine) and contraception adherence check required for all WOCBP from randomization until 1 month after last dose. If positive, the treatment should be discontinued and procedures to be followed in the event of suspected contraceptive failure or suspicion of pregnancy. The company may consider options for pregnancy testing at home by a health care professional for months where no visit is scheduled.
^{d} Woman not of childbearing potential are to be confirmed at screening by fulfilling one of the following criteria: (a) postmenopausal defined as amenorrhea for at least 12 months or more following cessation of all exogenous hormonal treatments and FSH levels in the postmenopausal range in the absence of 12 months of amenorrhea, a single FSH measurement is insufficient to define postmenopausal criteria. In case of perimenopause or infrequent periods with variable levels of FSH, women should be considered of childbreaking potential; or (b) documentation of irreversible surgical sterilization by hysterectomy, bilateral oophorectomy, or bilateral salpingectomy but not tubal ligation.
^{e} Including signs of fluid retention and heart failure.
^{f} Tests to include: B-hemoglobin, B-leukocyte differential count (absolute count), B-platelet count, B-HbA1c, B-hematocrit, B-leukocyte count, B-BNP, B-red blood cell count, S-bicarbonate, S-chloride, S-GGT, S-glucose, S-albumin, S-ALP, S-ALT, S-AST, total S-bilirubin, S-BUN, total S-calcium, S-creatine kinase, S-creatinine (including eGFR assessment), S-magnesium, S-phosphorous, S-potassium, S-sodium.
^{g} Visit 11 mean eGFR value will be calculated from eGFR values at Visit 11 and Visit 11.1.
^{h} PK samples will be collected pre-dose at Visits 3, 6 and 9.
ⁱ Outside of Visits 3, 6 and 9, study intervention will be taken at home.
^{j} Plasma, serum, and urine samples for potential future biomarker research are optional.
AE = adverse event; AESI = adverse event of special interest; ALP = alkaline phosphatase; ALT = alanine transaminase AST = aspartate aminotransferase; B = blood; BNP = B-type natriuretic peptide; BP = blood pressure; CV = cardiovascular; DAE = adverse event leading to discontinuation of investigational medicinal product; eGFR = estimated glomerular filtration rate; EoT = end of treatment; ECG = electrocardiogram; EQ-5D-5L = European Quality of Life 5-dimensional 5-level questionnaire; EQ-VAS = European Quality of Life Visual Analogue Scale; FSH = follicle stimulating hormone; GGT = gamma-glutamyl transferase; HbA1c = glycated hemoglobin; HIV = human immunodeficiency virus; NYHA = New York Heart Association; PK = pharmacokinetic; PRO = patient-reported outcome; PTDV = premature treatment discontinuation visit; Q4M = every 4 months; QD = once daily; S = serum; SAE = serious adverse event; S-BUN = S-blood urea nitrogen; SGLT2i = sodium-glucose co-transporter 2 inhibitor; SPFQ = Study Participant Feedback Questionnaire; UACR = urine albumin to creatinine ratio; UPCR = urine protein to creatinine ratio; WOCBP = women of childbearing potential.

### Study Compliance

Participants will take treatment at the clinic on indicated visit days and at home on all other occasions. When participants are dosed at the site, they will receive study intervention(s) directly from the investigator or designee, under medical supervision. The date, and time if applicable, of dose administered in the clinic will be recorded in the source documents and recorded in the electronic case reporting form (eCRF). The dose of study intervention and study participant identification will be confirmed at the time of dosing by a member of the study site staff other than the person administering the study intervention. When participants self-administer study intervention(s) at home, compliance with study intervention will be assessed at each visit.

Compliance will be assessed by direct questioning, counting returned tablets during the site visits and documented in the source documents and eCRF. Deviation(s) from the prescribed dosage regimen will be recorded in the eCRF.

A record of the quantity of the zibotentan/dapagliflozin 0.25 mg/10 mg and zibotentan/dapagliflozin 0.75 mg/10 mg FDC, and dapagliflozin 10 mg tablets dispensed to and administered by each participant will be maintained and reconciled with study intervention and compliance records. Intervention start and stop dates, including dates for intervention delays and/or dose reductions will also be recorded in the eCRF.

### Dose Modification

Participants who were randomized to either high-dose FDC (zibotentan/dapagliflozin 0.75 mg/10 mg) or matching dapagliflozin monotherapy may be down-titrated to low-dose FDC (zibotentan/dapagliflozin 0.25 mg/10 mg) or have sham down-titration if on dapagliflozin monotherapy, after consultation with the medical monitor including the following reasons (double-blinding is maintained):
Signs and/or symptoms of fluid retention assess to be related to the investigational medicinal product (IMP) that does not resolve with interventions.
Symptomatic hypotension not resolved with adjustment of concomitant anti-hypertensive medication.

Additionally, any participant who was randomized to either high-dose FDC (zibotentan/dapagliflozin 0.75 mg/10 mg) or matching dapagliflozin monotherapy who has an eGFR decline to <30 mL/min/1.73 m², confirmed by central laboratory either at a scheduled or unscheduled study visit, will be down-titrated to low-dose FDC (zibotentan/dapagliflozin 0.25 mg/10 mg) or have sham down-titration if on dapagliflozin monotherapy, regardless of symptoms (double-blinding is maintained).

As these conditions are known to occur in patients with CKD and high proteinuria, clinical judgment should be used to determine if it is related to medical history or an adverse event (AE).

There will be no dose modification for participants on low-dose FDC (zibotentan/dapagliflozin 0.25 mg/10 mg) or matching dapagliflozin monotherapy. However, IMP dose(s) may be held when clinically necessary (applicable for both high and low dose levels). In the event of a missed or delayed dose for any reason, the subsequent dose should be administered according to the original schedule (i.e., at the planned time point relative to the first dose of IMP).

### Overdose

Dapagliflozin has been well tolerated at doses up to 500 mg/day in single dose testing in healthy volunteers and up to 100 mg/day in repeat dose testing for 14 days in healthy volunteers and patients with type 2 diabetes. If an overdose is suspected, monitoring of vital functions as well as treatment will be performed as appropriate.

Presently there is no information regarding overdose of zibotentan in humans. For the purposes of this study, an overdose is defined as the use of study intervention in doses in excess of that specified in the protocol.

In the event of an overdose, the investigator/treating physician will:
Evaluate the participant to determine, in consultation with the Study Clinical Lead, if possible, whether study intervention should be interrupted or whether the dose should be reduced.
Closely monitor the participant for any AE/SAE and laboratory abnormalities.

### Concomitant Medications

Any medication or vaccine (including over the counter or prescription medicines, recreational drugs, vitamins, and/or herbal supplements) that the participant (except for screen failures) is receiving at the time of enrolment or receives during the study will be recorded along with:
Reason for use.
Dates of administration including start and end dates.
Dosage information including dose, frequency, and route.

As ET-1 has a potential role in the maintenance of blood pressure, hypotension may be encountered following ET blockade. Although only minor reductions in blood pressure were seen in patients it is possible that more marked changes might occur in hypertensive patients especially those taking hypotensive therapy such as ACE inhibitors, calcium antagonists, or alpha blockers. If symptomatic hypotension occurs, subjects will remain supine until resolution of symptoms. If persistent hypotension is observed, adjustment of concomitant medications for blood pressure control will be considered. Intravenous fluid support will be considered for cases considered severe by the Investigator.

Participants using medications that can cause hypoglycemia in T2DM patients, including insulin or sulfonylurea (SU), may be required to reduce insulin by 10% to 20% (total daily dose) and SU by 25% to 50%. In addition, more frequent blood glucose monitoring will be considered in participants receiving insulin and/or SU and with baseline HbA1c≤7% at randomization.

The medications and supplements listed below are prohibited from the time of consent and for the duration of the study. Participants taking any of these medications at the time of randomization will not be included into the study:
Strong or moderate CYP3A4 inducer
Cyclosporin or tacrolimus.
Receiving cytotoxic therapy, immunosuppressive therapy, or other immunotherapy for primary or secondary renal disease within 6 months prior to screening other than stable maintenance therapy (see exclusion criteria).
Tolvaptan, any other ERAs, or budesonide (where used to treat IBD or IgAN).

Additionally, the last dosing day of any SGLT2i will be the day before the randomization visit as all participants will be receiving the SGLT2i dapagliflozin as part of the IMP from randomization.

If a participant's medical condition requires rescue therapy, the participant will be treated, and withdrawn from study intervention administration at the investigator's discretion. Rescue therapy will be recorded in the eCRF.

Diabetic participants may require adjustment of their other glucose-lowering treatments on commencing blinded study intervention (due to the potential of dapagliflozin treatment) to avoid hypoglycemia.

Initiation and/or titration of diuretic therapy will be used for participants assessed with new or worsening signs/symptoms of fluid overload.

### Study Discontinuation

It may be necessary for a participant to permanently discontinue (definitive discontinuation) study intervention. An individual participant may be discontinued from study intervention in the following situations:
Participant decision. The participant is at any time free to discontinue treatment, without prejudice to further treatment.
An AE that, in the opinion of the investigator or Sponsor, warrants discontinuation from further dosing.
Severe non-compliance with the clinical study protocol (CSP).
Safety reasons as judged by the investigator and/or Sponsor where continued treatment may put participant at undue risk.

An individual participant will be discontinued from study intervention in the following situations:
If the participant becomes pregnant during the course of the study, study intervention should be discontinued immediately, and a Sponsor representative will be notified.
Diabetic ketoacidosis (DKA): DKA including euglycemic DKA have been reported with use of dapagliflozin. Will consider temporary interrupting study treatment if DKA is suspected. The participant will be promptly evaluated. If DKA is confirmed, study intervention will be discontinued permanently. If DKA is not confirmed, restart of study intervention will be considered.
Symptomatic hypotension (defined as systolic blood pressure<90 mmHg OR diastolic blood pressure<60 mmHg with symptoms) without reversible cause identified and adjustment of concomitant medications does not resolve symptoms.
Hospitalization for HF.
End-stage kidney disease.
Increase in systemic immunosuppression doses above those permitted at inclusion.

It is noted that discontinuation from study intervention is not the same thing as a withdrawal from the study. If study intervention is permanently discontinued, the participant will complete the premature treatment discontinuation visit, continue with the study visits, and complete further evaluations as per the schedule of activities.

Predisposing factors to ketoacidosis include a low beta cell function reserve resulting from pancreatic disorders (e.g., type 1 diabetes, history of pancreatitis or pancreatic surgery), insulin dose reduction, reduced caloric intake or increased insulin requirements due to infections, illness or surgery and alcohol abuse. Dapagliflozin will be used with caution in these participants.

Participants treated with dapagliflozin who present with signs and symptoms consistent with ketoacidosis, including nausea, vomiting, abdominal pain, malaise and shortness of breath will be assessed for ketoacidosis, even if blood glucose levels are below 14 mmol/L (250 mg/dL). If ketoacidosis is suspected, discontinuation or temporary interruption of study treatment will be considered, and the participant should be promptly evaluated.

Exposure during gestation to ERAs, including zibotentan, is associated with major embryo-fetal harm. Endothelin receptor antagonists have been demonstrated to induce teratogenic effects in animals when dosed during organogenesis, early in pregnancy. When administered to pregnant rabbits during the period of major embryonic organogenesis, zibotentan caused teratogenic effects at dose levels of 1 mg/kg/day and above, consistent with ERAs. Due to the established teratogenic effect, pregnant women or WOCBP who are not practicing reliable contraception methods will not receive zibotentan.

Dapagliflozin will not be used in the second and third trimesters of pregnancy due to the increased incidence and/or severity of renal pelvic and tubular dilatations in progeny. When pregnancy is detected, dapagliflozin will be discontinued. However, since zibotentan is teratogenic early in pregnancy, the more restrictive guidance for zibotentan will be followed for the combination.

There is an extremely high risk of embryo-fetal harm if pregnancy occurs while WOCBP take zibotentan in any amount, even for short periods of time. Potentially, any fetus exposed during pregnancy can be affected.

Pregnancy tests will be required monthly during treatment and one month after discontinuation of treatment with study intervention. All WOCBP participants must use adequate contraception (see inclusion criterion) prior to initiation of treatment, during treatment, and for one month after discontinuation of treatment with study intervention.

The investigators will educate and counsel the WOCBP participants on the embryo-fetal toxicity and the risk of unintended pregnancy. The investigators will assess the WOCBP participant's adherence with the required contraception methods during the study on a monthly basis and as per the schedule of activities.

In case of intercourse without the correct use of adequate contraception, the investigator must be immediately notified, and the study intervention will be interrupted. The participant will be made aware of the availability of emergency contraception (pills or intrauterine device) if the suspected failure occurred within the last 5 days. Emergency methods of contraception will be approved by relevant authorities in the participant's territory. The investigational treatment will be resumed after at least 4 weeks have passed since the suspected contraception failure, providing a negative highly reliable serum pregnancy test (S-hCG) is obtained, adequate pregnancy prevention is correctly used by the participant, and that resumption is judged to be appropriate by the investigator.

If suspicion of pregnancy emerges based on signs or symptoms of early pregnancy, such as a missed period, nausea, tiredness, moodiness, or sore breasts, the participant will notify the investigator and perform unscheduled pregnancy testing. The participant will be promptly evaluated, a negative test confirmed with a highly reliable serum test (S-hCG), and the study intervention will be interrupted until pregnancy is reliably excluded.

In the event of a positive urine test the study intervention will be interrupted, and the pregnancy promptly confirmed using a highly reliable serum test (S-hCG).

In the case of confirmed pregnancy, the study intervention will be discontinued, and the investigator will follow the emergency unblinding procedure and immediately notify a Sponsor representative. Access to diagnostic tests will be provided to determine pregnancy duration and reveal any detectable fetal anomalies. The participant will be provided counselling considering the risks associated with the participant's underlying medical condition, teratogenicity of the received treatment, possible duration of inadvertent embryo-fetal exposure, and risk of complications. Participants will be guaranteed a medical follow-up. The participants will be offered the option to end their participation or to stay in the study for safety monitoring. The pregnancy will be followed and reported.

Discontinuation of the participant from the study by the investigator may occur as described below:
A participant may be discontinued from the study at any time at the discretion of the investigator for safety, behavioral, compliance, or administrative reasons.
At the time of discontinuing from the study, if the participant has not been discontinued from the study intervention.

Voluntary withdrawal from the study by the participant may occur as described below:
A participant may withdraw from the study at any time at the participant's own request for any reason (or without providing any reason).
A participant who wishes to withdraw from the study will be informed by the investigator about modified follow-up options (e.g., telephone contact, a contact with a relative or treating physician, or information from medical records).
If the participant withdraws consent for disclosure of future information, the Sponsor will retain and continue to use any data collected before such a withdrawal of consent.
If a participant withdraws from the study, it will be confirmed if he/she still agrees for existing samples to be used in line with the original consent. If he/she requests withdrawal of consent for use of samples, destruction of any samples taken and not tested will be carried out in line with what was stated in the informed consent and local regulation. The investigator will document the decision on use of existing samples in the site study records and inform the respective Site Monitor who then informs the Global Study Team.

A participant will be considered lost to follow-up if the participant repeatedly fails to return for scheduled visits and is unable to be contacted by the study site.

The following actions will be taken if a participant fails to return to the study site for a required study visit:
The site will attempt to contact the participant and reschedule the missed visit as soon as possible. The participant will be counseled on the importance of maintaining the assigned visit schedule. At this time ascertain whether the participant will or wishes to or continue in the study.
Before a participant is deemed lost to follow-up, the investigator or designee will make every effort to regain contact with the participant (where possible, at least 3 telephone calls, texts, emails, and if necessary, a certified letter to the participant's last known mailing address or local equivalent methods). These contact attempts will be documented in the participant's medical record.
Site personnel, or an independent third party, will attempt to collect the vital status of the participant within legal and ethical boundaries for all participants randomized, including those who did not get study intervention. Public sources will be searched for vital status information. If vital status is determined as deceased, this will be documented, and the participant will not be considered lost to follow-up. Sponsor personnel will not be involved in any attempts to collect vital status information.

### Objectives and Endpoints

Study objectives and endpoints are outlined in Table 8.

TABLE 8
Objectives and Endpoints.

| | |
|---|---|
| Objectives | Endpoints |

Primary

| | |
|---|---|
| To determine whether zibotentan and | Change in eGFR from baseline to Month |
| dapagliflozin in fixed-dose combination | 24. |
| is superior to dapagliflozin alone to slow | |
| decline in renal function. | |

Secondary

| | |
|---|---|
| To determine whether zibotentan and | Change from baseline in UPCR. |
| dapagliflozin in fixed-dose combination | |
| is superior to dapagliflozin alone in | |
| reducing proteinuria. | |
| To determine whether zibotentan and | Change from baseline in UACR. |
| dapagliflozin in fixed-dose combination | |
| is superior to dapagliflozin alone in | |
| reducing albuminuria. | |
| To determine whether zibotentan and | Time to the first occurrence of any of the |
| dapagliflozin in fixed-dose combination | components of the renal composite |
| is superior to dapagliflozin alone in | endpoint of 40% sustained decline in |
| reducing the incidence of the renal | eGFR or ESKD or renal death. |
| composite endpoint of 40% sustained | |
| decline in eGFR or ESKD or renal | |
| death. | |
| To determine whether zibotentan and | Change from baseline in systolic blood |
| dapagliflozin in fixed-dose combination | pressure. |
| is superior to dapagliflozin alone in | |
| reducing systolic blood pressure. | |
| To determine whether zibotentan and | Proportion of participants achieving |
| dapagliflozin in fixed-dosed combination | UPCR <1 g and >30% reduction from |
| is superior to dapagliflozin alone, in | baseline. |
| reducing proteinuria, as measured by | |
| the proportion of participants achieving | |
| UPCR <1 g and >30% reduction from | |
| baseline. | |

Safety

| | |
|---|---|
| To assess the safety and tolerability of | AEs, SAEs, SAEs with outcome of |
| treatment with zibotentan and | death, DAEs. |
| dapagliflozin in fixed-dose combination | Clinical laboratory |
| compared to dapagliflozin alone. | Vital signs including blood pressure, |
| | pulse rate and body weight. |
| | ECG. |
| | AESIs: heart failure, fluid retention, and |
| | hepatoxicity |

Exploratory

| | |
|---|---|
| To explore the effect of zibotentan and | Total eGFR slope |
| dapagliflozin in fixed-dose combination | Chronic eGFR slope |
| compared to dapagliflozin alone to slow | |
| decline in renal function, as measured | |
| by total and chronic eGFR slope. | |
| To explore the effect of zibotentan and | Proportion of participants with CKD |
| dapagliflozin in fixed-dose combination | stage 3 (eGFR ≥30 mL/min/1.73 m²) |
| compared to dapagliflozin alone on CKD | who enter CKD stage 4 (eGFR <30 |
| progression from stage 3 to stage 4. | mL/min/1.73 m²) at Month 24. |
| To explore the effect of zibotentan and | Time to the first occurrence of any of the |
| dapagliflozin in fixed-dose combination | components of the renal composite |
| compared to dapagliflozin alone in | endpoint of 50% sustained decline in |
| reducing the incidence of the renal | eGFR or ESKD or renal death. |
| composite endpoint of 50% sustained | |
| decline in eGFR or ESKD or renal | |
| death. | |
| To explore the effect of zibotentan and | Time to the first occurrence of any of the |
| dapagliflozin in fixed-dose combination | components of the renal composite |
| compared to dapagliflozin alone in | endpoint of 57% sustained decline in |
| reducing the incidence of the renal | eGFR or ESKD or renal death. |
| composite endpoint of 57% sustained | |
| decline in eGFR or ESKD or renal | |
| death. | |
| To explore the effect of zibotentan and | Proportion of participants who achieve |
| dapagliflozin in fixed-dose combination | either UACR <300 mg/g or a reduction |
| compared to dapagliflozin alone in | of at least 50% at Month 24. |
| increasing the proportion of participants | |
| with nephrotic range proteinuria who | |
| achieve either UACR <300 mg/g or a | |
| reduction of at least 50%. | |
| To explore the effect of zibotentan and | Proportion of participants with |
| dapagliflozin in fixed-dose combination | UACR <300 mg/g g/g at Month 24. |
| compared to dapagliflozin alone in | |
| increasing the proportion of participants | |
| achieving <300 mg/g UACR. | |
| To explore the effect of zibotentan and | Changes from baseline in health status |
| dapagliflozin in fixed-dose combination | measured by EQ-5D-5L (including EQ- |
| compared to dapagliflozin alone, on | VAS), at each of the post randomization |
| health status assessed by EQ-5D-5L, | visits up to and including Month 24. |
| including EQ-VAS, to support health | |
| economic analysis and health | |
| technology assessment. | |
| To explore the effect of zibotentan and | Time to the first occurrence of any of the |
| dapagliflozin in fixed-dose combination | following components of the CV |
| compared to dapagliflozin alone in | composite endpoint of CV death or MI or |
| reducing the incidence of the CV | stroke. |
| composite endpoint of CV death or MI or | |
| stroke. | |
| To explore the effect of zibotentan and | Time to the first occurrence of any of the |
| dapagliflozin in fixed-dose combination | components of the composite endpoint |
| compared to dapagliflozin alone in | of stroke or acute coronary syndrome. |
| reducing the incidence of the composite | |
| endpoint of stroke or acute coronary | |
| syndrome. | |
| To characterize the plasma exposure of | Plasma concentration per treatment |
| zibotentan and dapagliflozin. | group and study visit. |

AE = adverse event; AESI = adverse event of special interest; CV = cardiovascular; DAE = AEs leading to discontinuation of IMP; ECG = electrocardiogram; eGFR = estimated glomerular filtration rate; ECG = electrocardiogram; EQ-5D-5L = European Quality of Life 5-dimensional 5-level questionnaire; EQ-VAS = VAS = European Quality of Life Visual Analogue Scale; ESKD = end-stage kidney disease; IMP = investigational medicinal product; MI = myocardial infarction; SAE = serious adverse event; UACR = urine albumin to creatinine ratio; UPCR = urine protein to creatinine ratio.

Potential endpoint events will be identified through laboratory data (see the schedule of activities (Tables 7A-7C) for laboratory assessments and timings), when questioning the participant about his/her overall health, or through information received through standard medical practice. Investigators will be encouraged to have a low threshold to submit any potential/possible event that might represent an endpoint.

The following potential endpoint events will be recorded in the eCRF and submitted for central adjudication:
Death
Heart failure (adverse event of special interest (AESI))

For each potential endpoint event, the investigator or delegate will record the endpoint specific information the eCRF. If the event is subject to adjudication, relevant source documents will be assembled. The source documents and relevant eCRF data will be sent for central adjudication.

Detailed instructions regarding endpoint reporting will be provided to the study sites. The clinical event adjudication (CEA) committee will not possess any governance authority, and will be blinded to randomized group. Additional details about the evaluations of potential endpoint events will be described in the CEA charter. Potential endpoint events will also be reported as AEs/SAEs.

Primary Variable: eGFR

Following the current recommendations, race will be omitted from calculations of eGFR. (Error! Reference source not found., Error! Reference source not found.). In this study, the lower bound of eGFR (eGFR≥20 ml/min) is set as a limit where participants may progress but still have time for eGFR decline before an event of ESKD occurs. Upper bound for CKD (eGFR<90 ml/min) is set with regards to benefit/risk. Participant eGFR will be calculated according to the 2021 CKD-EPI equation, based on

sCr concentration alone (Error! Reference source not found.):

$$eGFR 1 = 142 \times {min ( {S}_{cr} / κ , 1 )}^{- 0.241 ( if female ) / - 0.302 ( if male )} \times {max ( {S}_{cr} / κ , 1 )}^{- 1.2} \times {0.994}^{Age} \times 1.012 [ if female ]$$

Where:
SCr=serum creatinine (mg/dL)
K=0.7 for females; and 0.9 for males
min=minimum of Scr/K or i
max=maximum of Scr/K or 1

Blood samples for the determination of serum creatinine and calculation of estimated eGFR will be collected at the time points described in the schedule of activities (Tables 7A-7C) and analyzed centrally.

### Secondary Variable: UACR

The UACR is a key marker for assessing kidney function. The UACR is a ratio between 2 measured substances (urine albumin and creatinine), which estimates 24-hour urine albumin excretion. The UACR is calculated as follows:

UACR (mg/g)=urine albumin (mg/dL)/urine creatinine (g/dL)

Urine samples for the determination of albumin and creatinine levels and calculation of UACR will be collected at the time points described in the schedule of activities (Tables 7A-7C) and analyzed centrally. The samples will also be used for the determination of exploratory urinary parameters.

### Secondary Variable: UPCR

The UPCR is a key marker for assessing kidney function. The UPCR is a ratio between 2ºmeasured groups of substances (urine proteins and creatinine), which estimates 24-hour urine protein excretion. The UPCR is calculated as follows:

$$UPCR ( mg / g ) = urine proteins measured by the assay ( mg / dL ) / urine creatinine ( g / dL )$$

Urine samples for the determination of proteins and creatinine levels and calculation of UPCR will be collected at the time points described in the schedule of activities (Tables 7A-7C) and analyzed centrally.

### Secondary Variable: Renal Composite Endpoint

A secondary objective of the study is to determine whether zibotentan and dapagliflozin in FDC is superior to dapagliflozin alone in reducing the time to first occurrence of any of the components of the renal composite endpoint of 40% sustained decline or ESKD or renal death.

Endpoints Related to eGFR Decline.

Laboratory values related to eGFR decline will trigger an action by site in the following situations:
If local laboratory values are obtained outside of the study procedures and indicate that eGFR value has declined≥40% compared with baseline or is below 10 mL/min/1.73 m².
   As soon as possible, the participant will come to the study site to confirm the local laboratory value with central laboratory testing.
OR
Central laboratory values, collected during a study visit, indicating that eGFR value has declined≥40% compared with baseline, or is below 10 mL/min/1.73 m².

The central laboratory will notify site if eGFR is <10 mL/min/1.73 m² or if there is ≥40% decline in eGFR compared to baseline. A re-sampling will be done at an unscheduled visit after at least 4 weeks, and preferably no later than 6 weeks after the first sampling. When a central laboratory value is <10 mL/min/1.73 m² or if there is ≥40% decline in eGFR compared to baseline it will be recorded as a potential endpoint in the eCRF.

The central laboratory will calculate eGFR using CKD-EPI equation based on sCr concentration alone (Inker et al 2021).

### Dialysis and Renal Transplantation

If a participant starts dialysis and or goes through a renal transplantation this will be recorded in the eCRF. ESKD is defined as:
Sustained eGFR<10 mL/min/1.73 m² or,
Chronic dialysis treatment (Dialysis ongoing for more than 28 days) or,
Receiving a renal transplant

### Renal Death

The CEA committee members will adjudicate and classify all deaths based on definitions described in the CEA charter. Renal death is defined as death due to ESKD when dialysis is not given. The investigator will record the classification of renal death in the eCRF.

### Exploratory Variable: Patient Reported Outcomes (EuroQol Five-Dimensional Five-Level Questionnaire)

Patient reported outcome (PRO) is an umbrella term referring to all outcomes and symptoms that are directly reported by the participant. PROs have become important endpoints for regulatory and reimbursement authorities when evaluating effectiveness of treatments in clinical trials. The following PROs will be administered in the study: EQ-5D-5L (see Appendix A in Example 1). Participants will be asked to complete the EQ-5D-5L at the visits as specified in the schedule of activities (Tables 7A-7C).

The EQ-5D-5L is a self-reported questionnaire that is used to derive a standardized measure of health status, also referred to as a utility score. The EQ-5D-5L utility scores are widely accepted by reimbursement authorities and will be used to support health economic evaluations.

The PRO will be administered electronically. Randomized participants will complete the PRO assessments at the site using a handheld electronic device (ePRO). Each site will allocate the responsibility for the administration of the ePROs to a specific individual and, if possible, assign a backup person to cover if that individual is absent. All assessments will be completed as follows:
Participant will not receive help from relatives, friends, or site personnel to answer or clarify the PRO questionnaires, in order to avoid bias. If a participant uses visual aids (e.g., spectacles or contact lenses) for reading and does not have them at hand, the participant will be exempted from completing the PROs questionnaires on that visit.
Before any other study procedures are conducted at a given visit.
Before being seen by the investigator.
The PRO questionnaires will be completed by the participant in private.
The appointed site personnel will explain to the participant the value and relevance of ePRO assessments and inform that these questions are being asked to find out, directly from the participant, how he/she feels. The appointed site personnel will also stress that the information is confidential.
The appointed site personnel will show participants how to use the ePRO device, in accordance with the instructions provided.
The appointed site personnel will remind participants that there are no right or wrong answers, and the participant will be given sufficient time to complete the PRO questionnaires.
If the participant is unable to read the questionnaire (e.g., is blind or illiterate), the participant will be exempted from completing the PRO questionnaires and may still participate in the study.

### Safety

Safety and tolerability will be evaluated in terms of AEs, SAEs, DAEs, SAEs with outcome of death, AESIs (HF, fluid retention, and hepatotoxicity), clinical laboratory, vital signs, and ECG.

Planned time points for all safety assessments are provided in the schedule of activities (Tables 7A-7C).

### Physical Examinations

A full physical examination will include assessments of the following: general appearance, respiratory, cardiovascular, abdomen, skin, head and neck (including ears, eyes, nose, and throat), lymph nodes, thyroid, musculoskeletal (including spine and extremities), neurological systems, and volume status. A targeted physical examination will focus on volume status, including signs and symptoms of fluid retention and heart failure. Physical examination, as well as assessment of height, will be performed at the time points specified in the schedule of activities (Tables 7A-7C).

### Vital Signs

Vital signs will be performed at the time points specified in the schedule of activities (Tables 7A-7C).

### Pulse and Blood Pressure (BP)

Pulse and BP will be measured 3 times per time point, and all measurements will be recorded in the eCRF. The measurements will be taken before any blood sampling using a standardized calibrated cuff adapted to the size of the participant's arm after the participant has been sitting and resting for least 5 minutes in a quiet setting without distractions (e.g., television, cell phones). Preferably, the same arm will be used at all visits.

### Body Weight

The participant's body weight will be measured with light clothing and no shoes. If the participant has a prosthetic limb, this will be consistently worn or not worn during all weight measurements.

### Electrocardiograms

A 12-lead ECG (standard ECG with a paper speed of 25 to 50 mm/second covering at least 6 sequential beats) will be recorded at the visits outlined in the schedule of activities (Tables 7A-7C) after the participant has been resting in a supine position for at least 5 minutes.

### Clinical Safety Laboratory Tests

Blood and urine samples for determination of clinical chemistry, hematology, and urinalysis will be taken at the visits indicated in the schedule of activities (Tables 7A-7C).

Additional safety samples will be collected if clinically indicated at the discretion of the investigator. The date, time of collection and results (values, units, and reference ranges) will be recorded on the appropriate eCRF.

The clinical chemistry, hematology, and urine chemistry will be performed at a central laboratory. The date of central laboratory sample collection will be recorded in the eCRF.

Other safety laboratory tests include assessment for pregnancy (serum at screening or serum or urine at other time points), hepatitis B and C serology and HIV antibody test. At Visit 2 (randomization), the urine pregnancy test will be negative before first intake of study intervention.

All samples will be taken by adequately trained site personnel and handled as instructed. Up to date reference ranges will be provided during the study and laboratory results will be compared with the laboratory standard normal ranges and reported back to site.

Samples sent to the central laboratory will be collected, labelled, stored and shipped as instructed.

The Investigator will make an assessment of the laboratory results with regards to clinically relevant abnormalities. The laboratory results will be signed and dated and retained at the study site as source data for laboratory variables. Laboratory safety variables are outlined in Table 8.

TABLE 8
Laboratory Safety Variables.

Hematology/Hemostasis (whole blood)
Laboratory Safety Variables.
Hematology/Hemostasis (whole blood)
B-Red blood cell (RBC) count
B-Hematocrit
B-Haemoglobin (Hb)
B-Leukocyte differential count (absolute count)
B-Leukocyte count
B-Platelet count
Urine Chemistry
U-Protein (including UPCR)
U-Albumin (including UACR)
U-Creatinine
Clinical Chemistry (serum or plasma)
S-albumin
S-Alanine transaminase (ALT)
S-Alkaline phosphatase (ALP)
S-Aspartate transaminase (AST)
S-Bicarbonate
S-Bilirubin, total
S-Blood urea nitrogen (BUN)
S-Calcium, total
S-Chloride
S-Gamma-glutamyl transferase (GGT)
S-Glucose
S-Creatine (including eGFR assessment)
S-Creatine kinase
S-Magnesium
S-Phosphorus
S-Potassium
S-Sodium
B-type natriuretic peptide (BNP)
B-hemoglobin A1c (HbA1c)
Note Bene:
In case a participant shows an AST or ALT ≥ 3 × ULN together with TBL ≥ 2 × ULN, see Error! Reference source not found., for further instructions.

### Adverse Events (AE)

Participants (or, when appropriate, a caregiver, surrogate, or the participant's legally authorized representative) will notify the investigator or designees of symptoms associated with AE and SAE. These will then be assessed by the investigator and if considered an AE or SAE, it will be reported by the investigator.

The investigator and any designees are responsible for detecting, documenting, and recording events that meet the definition of an AE or SAE (see Table 9)

TABLE 9
AE and SAE Variables Collected and Reported.

| | Significant Adverse Event |
|---|---|
| Adverse Event Variables Collected | Variables Collected |
| AE (verbatim) | Date AE met criteria for SAE |
| The date when the AE started and | Date investigator became aware |
| stopped | of SAE |
| Maximum intensity | AE description |
| Whether the AE is serious or not | AE is serious due to |
| Investigator causality rating against | Date of hospitalization |
| the IMP(s) (yes or no) | |
| Action taken with regard to IMP(s) | Date of discharge |
| AE caused participant's withdrawal | Probable cause of death |
| from the study (yes or no) | |
| Outcome | Date of death |
| | Autopsy performed |
| | Causality assessment in relation to |
| | study procedure(s) |
| | Causality assessment to other |
| | medication |

### Adverse Events of Special Interest (AESI)

Additional monitoring and data collection will be performed during the study for the following safety events classified as AESIs, based on the potential risks identified for zibotentan (HF and fluid retention) in oncology clinical studies conducted previously. Although hepatotoxicity has been highlighted as risk associated with some other ERAs, it has not been observed with zibotentan so far:
New diagnosis of HF or worsening of the existing HF condition: Any treatment-emergent symptoms suggestive of HF should be properly assessed by the investigator, to ensure timely and proper management of the participant's condition.
Other signs of fluid retention or worsening of fluid retention (e.g., fluid retention, fluid overload, hypervolemia, oedema of any location, ascites, effusions, etc.).
Hepatotoxicity that includes possible drug-related hepatic disorders (e.g., hepatic failure, non-infectious hepatitis, DILI, other liver related signs and symptoms, etc.).

Adverse events will be collected from randomization throughout the treatment period and including the safety follow-up period. Serious AEs will be recorded from the time of signing of the informed consent form (ICF).

If the investigator becomes aware of an SAE with a suspected causal relationship to the investigational medicinal product that occurs after the end of the clinical study in a treated participant, the investigator will, without undue delay, report the SAE to Sponsor.

Any AEs that are unresolved at the participant's last AE assessment in the study will be followed up by the investigator for as long as medically indicated, but without further recording in the eCRF. Sponsor retains the right to request additional information for any participant with ongoing AE(s)/SAE(s) at the end of the study, if judged necessary.

The investigator will assess causal relationships between IMP and each AE, and answer ‘yes’ or ‘no’ to the question ‘Do you consider that there is a reasonable possibility that the event may have been caused by the IMP?’

For SAEs, causal relationship will also be assessed for other medication and study procedures. Note that for SAEs that could be associated with any study procedure the causal relationship is implied as ‘yes’.

Deterioration as compared to baseline in protocol-mandated laboratory values, vital signs, and ECG parameters will only be reported as AEs if they meet any of the following:
Fulfil any of the SAE criteria.
Are the reason for discontinuation of the IMP.
Are clinically relevant as judged by the investigator (which may include but is not limited to consideration as to whether intervention or non-planned visits were required or other action was taken with the IMP, e.g., dose adjustment or drug interruption).

If deterioration in a laboratory value/vital sign is associated with clinical signs and symptoms, the sign or symptom will be reported as an AE and the associated laboratory result/vital sign will be considered as additional information. Wherever possible the reporting investigator will use the clinical, rather than the laboratory term (e.g., anemia vs low hemoglobin value). In the absence of clinical signs or symptoms, clinically relevant deteriorations in non-mandated parameters will be reported as AE(s).

Any new or aggravated clinically relevant abnormal medical finding at a physical examination as compared with the baseline assessment will be reported as an AE.

All signs or symptoms spontaneously reported by the participant or reported in response to the open question from the study site staff: ‘Have you had any health problems since the previous visit/you were last asked?’, or revealed by observation will be collected and recorded in the eCRF.

When collecting AEs, the recording of diagnoses is preferred (when possible) to recording a list of signs and symptoms. However, if a diagnosis is known and there are other signs or symptoms that are not generally part of the diagnosis, the diagnosis and each sign or symptom will be recorded separately.

Cases where a participant shows elevations in liver biochemistry may require further evaluation, and occurrences of aspartate aminotransferase/transaminase (AST) or alanine transaminase (ALT)≥3×the upper limit of normal (ULN) together with total bilirubin (TBL)≥2×ULN may need to be reported as SAEs.

All SAEs will be reported whether or not considered causally related to the IMP. All SAEs will be recorded in the eCRF. If any SAE occurs during the study, investigators or other site personnel will inform the appropriate Sponsor representatives within one day, i.e., immediately but no later than 24 hours of when he or she becomes aware of it.

The designated Sponsor representative will work with the investigator to ensure that all the necessary information is provided to the Sponsor Patient Safety data entry site within one calendar day of initial receipt for fatal and life-threatening events and within 5 calendar days of initial receipt for all other SAEs.

For fatal or life-threatening AEs where important or relevant information is missing, active follow-up will be undertaken immediately. Investigators or other site personnel will inform Sponsor representatives of any follow-up information on a previously reported SAE within one calendar day, i.e., immediately but no later than 24 hours of when he or she becomes aware of it.

Once the investigators or other site personnel indicate an AE is serious in the Electronic Data Capture (EDC) system, an automated email alert is sent to the designated Sponsor representative.

If the EDC system is not available, then the investigator or other study site staff will report the SAE via secure method to the appropriate Sponsor representative.

When the EDC is temporarily not accessible, the Sponsor Study Representative should confirm that the investigator/site staff enters the SAE in the Sponsor EDC when access resumes.

### Sample Collection

Blood samples will be collected pre dose for measurement of plasma concentrations of zibotentan and dapagliflozin as specified in the schedule of activities (Tables 7A-7C).

Samples may be collected at additional time points during the study if warranted and agreed upon between the investigator and Sponsor, e.g., for urgent safety reasons; these samples may not be part of the pharmacokinetic (PK) analysis set and reporting. The timing of sampling may be altered during the study based on newly available data (e.g., to obtain data closer to the time of peak or trough matrix concentrations) to ensure appropriate monitoring.

Plasma samples will be used to analyze the PK of zibotentan and dapagliflozin. Samples collected for analyses of zibotentan and dapagliflozin plasma concentration may also be used to evaluate safety or efficacy aspects related to concerns arising during or after the study.

PK samples will be disposed of after the Bioanalytical Report finalization or 6 months after issuance of the draft Bioanalytical Report (whichever is earlier), unless consented for future analyses.

Additional analyses may be conducted on the anonymized, pooled, or individual PK samples to further evaluate and validate the analytical method. Any results from such analyses may be reported separately from the clinical study report (CSR).

Samples for determination of zibotentan and dapagliflozin concentration in plasma will be assayed by bioanalytical test sites operated by or on behalf of Sponsor, using an appropriately validated bioanalytical method. Zibotentan and dapagliflozin plasma concentrations will be measured separately.

Drug concentration information that would unblind the study will not be reported to investigative sites or blinded personnel until the study has been unblinded. Incurred sample reproducibility analysis, if any, will be performed alongside the bioanalysis of the test samples. The results from the evaluation, if performed, will be reported in a separate Bioanalytical Report.

### Study Procedures

### Visit 1 (Screening; all Participants) and Visit 1.1 (SGLT2i-Naïve Run-in Participants Only)

At enrollment, following provision of signed informed consent, the screening assessments and procedures will be performed as described per the schedule of activities for SGLT2i-naïve participants (Table 7A) and for SGLT2i-treated participants (Table 7B). After investigator review of the inclusion and exclusion criteria, including laboratory assessments and confirmation of a negative serum pregnancy test, further procedures will continue.

Participants who are SGLT2i-naïve who meet all inclusion/exclusion criteria, including a negative serum pregnancy test, will enter a 28-day run-in period of SGLT2i therapy. The specific SGLT2i during the run-in will be chosen by the investigator and will be provided as auxiliary drug either prescribed by the investigator or sourced locally and will be dose indicated for CKD. The 28-day run-in period will begin such that the last dose taken of the SGLT2i occurs the day prior to Visit 2 (i.e., Day −1). During the run-in period, participants will return to clinic for Visit 1.1, 3 days prior to Visit 2, for laboratory assessments. These laboratory assessments will be for baseline measurements and are not needed to further determine inclusion or exclusion to the study.

Participants who are SGLT2i-treated patients and who meet all inclusion and exclusion criteria, including a negative serum pregnancy test, will proceed to Visit 2 without needing the run-in or Visit 1.1 assessment. The last dose of their SGLT2i occurs the day prior to Visit 2 (i.e., Day −1).

### Visit 2 (Randomization)

Participants will discontinue any previous SGLT2i therapy the day prior to Visit 2 (i.e., Day −1). All assessments and procedures will be performed per the schedule of activities (Table 7A for SGLT2i-naïve and Error! Reference source not found. 7B for SGLT2i-treated participants). The investigator will review eGFR value obtained at Visit 1 to determine the dosing assignment. The study intervention will be dispensed to the participant via interactive responsive technology (IRT)/randomization and trial supply management (RTSM), and the participant will be instructed to take the study intervention in accordance with the protocol without interruption, with the first dose taken in clinic after completion of the assessments and procedures for Visit 2.

### Monthly Urine Pregnancy Test and Contraception Adherence Check (WOCBP Only)

Female participants of childbearing potential will have monthly urine pregnancy tests and contraception adherence checks as described in the schedule of activities from randomization until 1 month after last dose. The results of the urine pregnancy tests will be reviewed with the participant as well as a thorough review of adherence to contraception requirements. In case of suspected contraception failure, or suspected or confirmed pregnancy, the procedures described herein will be followed to determine whether the participant will continue or be withdrawn from the study.

### Premature Treatment Discontinuation Visit

If study intervention is permanently discontinued, the participant will complete the premature treatment discontinuation visit (PTDV), continue with the study visits and assessments as per the schedule of activities (Table 7C). The PTDV visit will occur within 28 days after the last dose, after which the next visit will be in accordance to schedule of activities, based on the last visit prior to the discontinuation of study intervention. Participants will continue in the study and receive open label dapagliflozin 10 mg monotherapy until the EoT visit, unless, in the opinion of the investigator, the participant is not able to tolerate dapagliflozin. The dapagliflozin 10 mg monotherapy will be an auxiliary drug either prescribed by the investigator or sourced locally.

### End of Treatment Visit

The EoT visit will occur after the CTED is reached. The CTED is defined as 2 years after the date of randomization of the last participant in the study and will be used to determine when the EoT visit should be scheduled. The EoT visits for all participants will occur preferably within 14 days and not more than 28 days after the CTED is declared. At the EoT visit, all assessments and procedures will be performed, as described in the schedule of activities (Table 7C). Participants will stop blinded study intervention and start open-label dapagliflozin 10 mg monotherapy until the safety follow-up visit (last study visit). The open label dapagliflozin 10 mg monotherapy will be an auxiliary drug either prescribed by investigator or sourced locally.

For participants who have prematurely discontinued study intervention but remained in the study as per the schedule of activities, the EoT visit will be the final study visit. After the final study visit, participants will return to their usual treatments per the standard of care at the discretion of the investigator. This includes treatment with SGLT2i (including dapagliflozin), if considered clinically indicated by the investigator or participant's treating physician.

### Safety Follow-Up Visit

The safety follow-up visit will occur 30 days after the EoT visit. At the safety follow-up visit, all assessments and procedures will be performed, as described in the schedule of activities (Table 7C). This will be the final study visit for participants in the study (except in cases of premature discontinuation as described above). After the final study visit, participants will return to their usual treatments at the discretion of the investigator. This includes treatment with SGLT2i (including dapagliflozin), if considered clinically indicated by the investigator or participant's treating physician.

### Statistical Considerations

All personnel involved with the analysis of the study will remain blinded until database lock and protocol violations have been identified and documented. An independent Data Monitoring Committee (DMC) will monitor data (unblinded) periodically during the conduct of the study. The remit of the DMC will be detailed in a DMC charter.

Assuming a true difference in change from baseline eGFR of 2.0 mL/min/1.73 m² between treatment groups, with assumed standard deviation of 15.5 using a two-sided alpha level of 5%, 1500 participants will provide a statistical power of 90%.

### Primary Analysis:

The primary efficacy variable of change from baseline in eGFR at Month 24 will be assessed through an estimand defined by following attributes:

Population: Patients with CKD and high proteinuria, with or without T2DM, and who meet eligibility criteria as defined by the inclusion and exclusion criteria.

Treatment: Zibotentan/dapagliflozin FDC QD or dapagliflozin (active comparator) QD.

### Intercurrent Events:

Premature study treatment discontinuation, change in IMP dosage, and change in concomitant medication: Treatment policy strategy, implemented by including all available data.
RRT or renal death: Composite strategy, implemented by defining eGFR=0 for the Month 24 visit, whenever RRT or renal death occurred at an earlier time point.
Non-renal death: Hypothetical strategy, implemented by handling the framework for missing data.

Population-level summary: Difference between treatment groups in mean change from baseline to post treatment at Month 24.

The main estimator for the primary endpoint is obtained by a mixed models for repeated measures (MMRM). The change in eGFR from baseline to the Month 24 visit is the dependent variable; all intermediate visits will be included in the MMRM analysis. Baseline is defined as the mean of 2 eGFR measurements on or prior to randomization. The model includes baseline eGFR as a continuous variable, and the stratification factors, treatment group, visit, and treatment-by-visit interaction as fixed effect factors.

Measurements after initiation of dialysis will not be included in the analysis. Missing post baseline eGFR assessments are implicitly handled in the likelihood-based parameter estimation assuming values in subjects with missing data are similar to those in subjects with observed data with the same treatment assignment and covariate values (i.e., missing at random).

The analysis will focus on the treatment effect on irreversible disease progression. To this end, estimates for the change from baseline (mean of the 2 last values obtained prior to randomization) to the mean of the 2 values obtained at the Month 24 and subsequent visits will be utilized. Least squares mean estimates will be presented for each treatment group, with 95% confidence intervals, and the difference between treatment groups, with 95% confidence intervals as well as the p-value.

### Secondary Analysis

The secondary endpoints of change from baseline in UACR, UPCR, and systolic blood pressure will be analyzed using a methodology similar to the one described for primary endpoint.

The secondary endpoint evaluating the renal composite endpoint of 40% sustained decline or ESKD or renal death will be analyzed with time-to-event using a cox proportional hazard model with factor for treatment, stratified by the randomization factor, and adjusted for eGFR at baseline as a continuous variable.

### Safety Analysis

Safety and tolerability will be evaluated in terms of AEs, vital signs, clinical laboratory variables, and ECGs. In addition, extra evaluation will be done for the following AESIs: HF; fluid retention; hepatoxicity.

The aim with the analyses of safety data is to assess the general safety objective, evaluated in a scenario where study treatment is not prematurely discontinued. Two analysis approaches are considered complementary to assess this hypothetical scenario for all safety endpoints:
On-treatment; unbiased under the assumption that censoring following premature treatment discontinuation is non-informative.
On-study; unbiased under the assumption that the risk for an event is independent of continued treatment. Note that this approach could also be described as handling premature treatment discontinuation with a treatment policy approach.

Hence, the general safety objective will be assessed through an estimand defined by the following attributes:

Population: Patients with CKD and high proteinuria, with or without T2DM, and who meet eligibility criteria as defined by the inclusion and exclusion criteria.

Treatment: Zibotentan/dapagliflozin FDC QD or dapagliflozin (active comparator) QD.

Intercurrent events (ICE):
Premature study treatment discontinuation: All analyses will be provided using 2 complementary approaches, unless otherwise stated:
   Hypothetical as if this ICE cannot happen, implemented by excluding/censoring data after premature study treatment discontinuation plus 28 days, using the on-treatment analysis period.
   Treatment policy by ignoring this ICE, implemented by including all available data, using the on-study analysis period.
Initiation or change of concomitant medication, including rescue medication: These ICEs will be ignored, that is, handled using a treatment policy approach.
Death: This ICE will be handled hypothetically as if it cannot happen, implemented by censoring time to event data at death and not imputing any data after death.

### Population Level Summaries and Estimators for Respective Analysis:

Probability of the event (e.g., AE, laboratory abnormality) occurring before a pre-defined time point. Difference in these between the treatment arms, as well as the HR (over the whole period of interest). For repeated measurements (e.g., laboratory evaluations performed at several time points) the distributions of the measurements at the respective time point.

This estimand requires that to the extent possible, randomized participants, included in the analysis set used for safety, are followed up regardless of study intervention compliance and adherence to the study protocol (for planned treatment period and safety follow-up).

Analysis: Hazard ratio comparing treatment groups will be estimated based on a Cox proportional hazards regression model with a factor for treatment group, using the full defined analysis period (on-study and on-treatment respectively). The cumulative distribution function (CDF) of time to event of interest in the respective treatment group will be estimated by a Kaplan-Meier estimator. The probability of the event of interest occurring before predefined time point will be estimated by the Kaplan-Meier estimator at one or more time points to be determined based on the extent of participant's follow-up (to be updated in the statistical analysis plan prior to clinical data lock (CDL) by treatment group for the defined analysis period (on-study and on-treatment respectively), and the treatment group difference will be estimated at one of the selected time points (selected prior CDL).

Comparisons between treatment groups will be presented with their associated 95% confidence interval. Since all safety analyses are descriptive, no p-values will be presented, and the confidence intervals are provided as illustration of precision of the estimate.

The on-study analysis period starts on the date of randomization visit and ends on the date of last clinical event assessment, unless the participant dies while under follow-up, then the date of death is taken as the end of the period. If a participant withdraws consent to continue in the study, then the on-study analysis period ends on this date at the latest.

The on-treatment analysis period starts on the date of randomization visit and ends on the earliest of 28 days following the date of last dose of IMP or the end of the On-study analysis period.

### Example 3

The safety and efficacy of a combination of zibotentan and dapagliflozin has been assessed in patients having chronic kidney disease with an eGFR≥20 mL/min/1.73 m², and a UACR≥150 mg/g and ≤5000 mg/g. Two combinations of zibotentan and dapagliflozin were evaluated where zibotentan was administered at a dose of 0.25 mg or 1.5 mg, and dapagliflozin was administered at a dose of 10 mg. The combinations of zibotentan and dapagliflozin were administered once daily over a 12-week period.

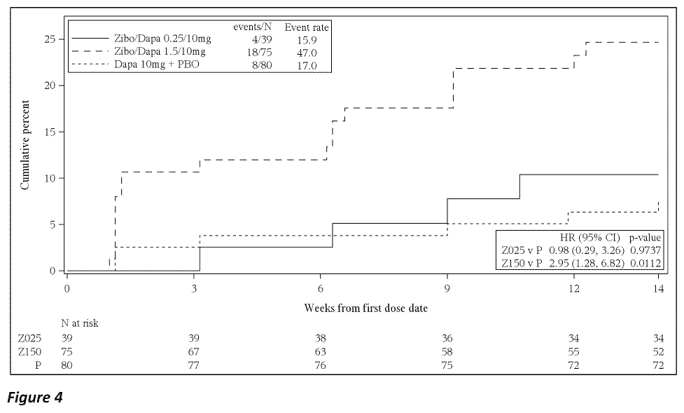

FIG. 4 depicts the change in fluid related events compared to baseline for study participants receiving dapagliflozin (10 mg; short dashed line), the low dose combination of zibotentan and dapagliflozin (0.25 mg/10 mg, respectively; solid line), and the high dose combination of zibotentan and dapagliflozin (1.5 mg/10 mg, respectively; long dashed line) in participants having a baseline UACR>700 mg/g. Only 4 out of a total of 39 subjects receiving the low dose combination of zibotentan and dapagliflozin (0.25 mg/10 mg) exhibited fluid related events, amounting to a relative event rate of 15.9%. The number of fluid related events and event rate was comparable to and not significantly different than participants receiving administration of dapagliflozin alone (8/80 events and a relative event rate of 17.0%). Study participants receiving the high dose of zibotentan and dapagliflozin (1.5 mg/10 mg) exhibited an increased relative event rate for fluid related events (47.0%) with 18/75 participants exhibiting events, which was significantly increased related to participants receiving dapagliflozin (10 mg) alone. These results indicate that the combinations of zibotentan and dapagliflozin are safe at least up to a dose of 1.5 mg/10 mg (zibotentan/dapagliflozin).

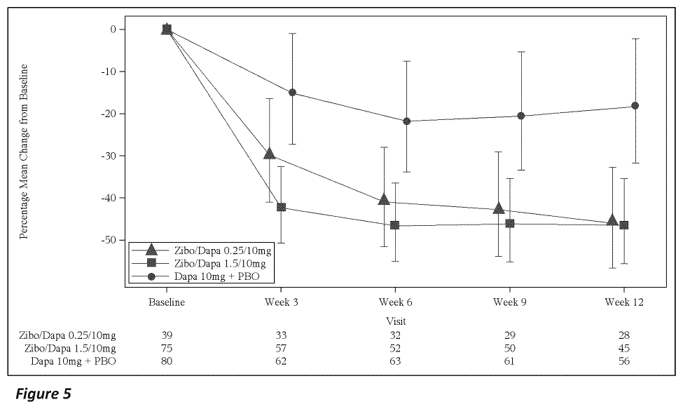

FIG. 5 depicts the UACR change over a 12-week period relative to baseline levels for study participants having a UACR>700 mg/g and receiving dapagliflozin (10 mg; green line with circle symbols), the low dose combination of zibotentan and dapagliflozin (0.25 mg/10 mg, respectively; blue line with triangle symbols), and the high dose combination of zibotentan and dapagliflozin (1.5 mg/10 mg, respectively; red line with square symbols). Over the 12-week period, participants receiving dapagliflozin alone exhibited a modest decrease in the mean percent change of UACR relative to baseline, with the percent change plateauing at an approximately 20% decrease from baseline. Participants receiving both the low dose (0.25 mg zibotentan and 10 mg dapagliflozin) and the high dose (1.5 mg zibotentan and 10 mg dapagliflozin) combinations exhibited marked decreases in UACR levels relative to baseline, with both the low-dose and high-dose treatment groups exhibiting changes in UACR levels at the end of the 12-week period that approximately 45% decreased relative to baseline levels. These results indicate that both tested doses of the combinations of zibotentan and dapagliflozin are effective at reducing UACR in individuals having high proteinuria (UACR>700 mg/g).

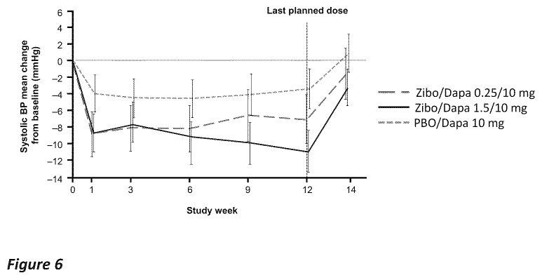

FIG. 6 depicts the change in systolic blood pressure over a 12-week administration period and 2-week post-administration follow up, relative to baseline levels, for study participants having a UACR<700 mg/g, as well as study participants having a UACR>700 mg/g. Over the 12-week period, participants receiving dapagliflozin+placebo (short dashed line; dapagliflozin 10 mg and placebo (PBO)) exhibited a modest decrease in systolic blood pressure—a decrease of 3.4% (90% Cl: −5.8, −1.0)—that was consistent over the course of the study. Following administration of the final planned dose, systolic blood pressure for participants receiving dapagliflozin+placebo returned to baseline levels, as measured at week 14. Participants receiving either dosage of the combination of dapagliflozin and zibotentan exhibited a more significant reduction in blood pressure as compared to baseline. Participants receiving the lower dose of the zibotentan and dapagliflozin combination (long dashed line; 0.25 mg zibotentan and 10 mg dapagliflozin) exhibited a mean decrease in systolic blood pressure from baseline of 7.1% (90% Cl: −10, −4.1), and participants receiving the higher dose of the zibotentan and dapagliflozin combination (solid line; 1.5 mg zibotentan and 10 mg dapagliflozin) exhibited a mean decrease in systolic blood pressure from baseline of 11.0% (90% Cl: −13.5, −8.4). The mean systolic blood pressure levels for participants receiving the combinations of zibotentan and dapagliflozin further remained below baseline levels two weeks after administration of the final planned dose. Results from this study regarding the systolic blood pressure measurements are further detailed in Table 10A and Table 10B. Table 10A. Systolic blood pressure measurement parameters.

TABLE 10A
Systolic blood pressure measurement parameters.

| | Number of participants assessed (n) | | | | | | |
|---|---|---|---|---|---|---|---|
| Treatment group | Week 0 | Week 1 | Week 3 | Week 6 | Week 9 | Week 12 | Week 14 |
| Zibo/Dapa (0.25/10 mg) | 91 | 84 | 83 | 75 | 68 | 65 | 74 |
| Zibo/Dapa (1.5/10 mg) | 179 | 154 | 143 | 133 | 124 | 108 | 141 |
| Dapa + placebo (10 mg) | 177 | 155 | 155 | 152 | 146 | 137 | 152 |

TABLE 10B
Impact of the combination of zibotentan and
dapagliflozin on systolic blood pressure.

| | Mean change | Difference in mean change versus |
|---|---|---|
| Treatment group | (90% CI) | dapagliflozin monotherapy (90% CI) |
| Zibo/Dapa | −7.1 | −3.6 |
| (0.25/10 mg) | (−10.0, −4.1) | (−6.8, −0.5) |
| Zibo/Dapa | −11.0 | −7.6 |
| (1.5/10 mg) | (−13.5, −8.4) | (−10.3, −4.9) |
| Dapa + placebo | −3.4 | — |
| (10 mg) | (−5.8, −1.0) | |

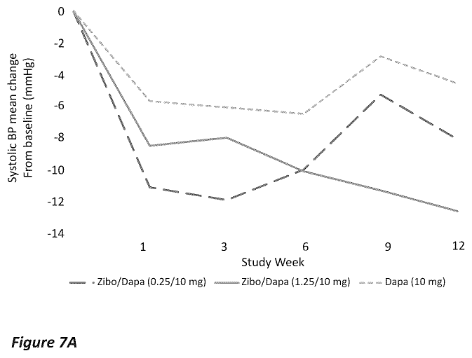

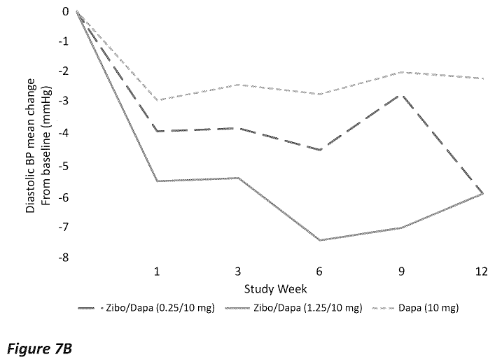

FIGS. 7A and 7B depict the change in systolic and diastolic blood pressure, respectively, for study participants having a UACR>700 mg/g over a 12-week administration period, relative to baseline levels. Over the 12-week period, study participants receiving dapagliflozin+placebo (short dashed line; dapagliflozin 10 mg and placebo) exhibit a modest decrease in systolic blood pressure and diastolic blood pressure, with a mean reduction in systolic blood pressure from baseline of just over 4%, and a mean reduction in diastolic blood pressure of approximately 2%, relative to baseline, at the end of the 12-week period. Patients receiving the lower dosage of the combination of zibotentan and dapagliflozin (long dashed line; 0.25 mg zibotentan and 10 mg dapagliflozin) exhibited a more significant decrease from baseline in systolic and diastolic blood pressure relative to those receiving dapagliflozin+placebo. A mean decrease from baseline systolic blood pressure of approximately 8% was recorded in the group receiving the low dose of the combination of zibotentan and dapagliflozin, and a mean decrease from baseline diastolic blood pressure of approximately 6% was recorded at the end of the 12-week study. For the higher dose of the zibotentan and dapagliflozin combination (solid line; 1.25 mg zibotentan and 10 mg dapagliflozin), a mean decrease from baseline of over 12% was recorded for systolic blood pressure, and a mean decrease of approximately 6% was recorded for diastolic blood pressure. The mean decrease in diastolic blood pressure was statistically significant for both the lower and higher dosage of the zibotentan and dapagliflozin combination relative to the dapagliflozin+placebo group (p=0.052 and p=0.024, respectively). The mean decrease in systolic blood pressure for the higher dosage of the zibotentan and dapagliflozin combination was also statistically significant relative to the group receiving dapagliflozin+placebo (p<0.001). Results from this study regarding systolic and diastolic blood pressure in patients having a UACR>700 mg/g are further detailed in Tables 11A and 11B, respectively.

TABLE 11A
Impact of the combination of zibotentan and dapagliflozin on systolic
blood pressure in participants having a UACR >700 mg/g.

| | n | | | | | |
|---|---|---|---|---|---|---|
| | Mean change from baseline (90% CI) | | | | | |
| | Mean difference vs Dapa (10 mg) (90% CI) | | | | | |
| | Zibo/Dapa | | Zibo/Dapa | | Dapa + placebo | |
| Visit | (0.25/10 mg) | | (1.25/10 mg) | | (10 mg) | |
| Base- | 144.8 | (17.60) | 139.8 | (16.05) | 140.7 | (15.68) |
| line | | | | | | |
| mean | | | | | | |
| (SD) | | | | | | |
| Week | 36 | | 63 | | 70 | |
| 1 | −11.1 | (−15.1, −7.0) | −8.5 | (−11.9, −5.1) | −5.7 | (−9.0, −2.4) |
| | −5.4 | (−9.9, −0.8) | −2.8 | (−6.6, 0.9) | | |
| Week | 35 | | 62 | | 71 | |
| 3 | −11.9 | (−16.2, −7.6) | −8.0 | (−11.6, −4.5) | −6.1 | (−9.5, −2.7) |
| | −5.8 | (−10.7, −0.9) | −2.0 | (−6.0, 2.1) | | |
| Week | 35 | | 56 | | 68 | |
| 6 | −10.0 | (−14.1, −5.9) | −10.1 | (−13.6, −6.6) | −6.5 | (−9.8, −3.1) |
| | −3.5 | (−8.1, 1.1) | −3.6 | (−7.6, 0.3) | | |
| Week | 31 | | 54 | | 65 | |
| 9 | −5.3 | (−9.6, −0.9) | −11.3 | (−14.9, −7.7) | −2.9 | (−6.4, 0.5) |
| | −2.3 | (−7.3, 2.6) | −8.4 | (−12.5, −4.2) | | |
| Week | 30 | | 46 | | 58 | |
| 12 | −8.1 | (−11.9, −4.2) | −12.6 | (−16.0, −9.2) | −4.6 | (−7.8, −1.5) |
| | −3.4 | (−7.8, 0.9)* | −8.0 | (−11.7, −4.2)** | | |

*p = 0.192,
**p < 0.001

TABLE 11B
Impact of the combination of zibotentan and dapagliflozin on diastolic
blood pressure in participants having a UACR >700 mg/g.

| | n | | | | | |
|---|---|---|---|---|---|---|
| | Mean change from baseline (90% CI) | | | | | |
| | Mean difference vs Dapa (10 mg) (90% CI) | | | | | |
| | Zibo/Dapa | | Zibo/Dapa | | Dapa + placebo | |
| | (0.25/10 mg) | | (1.25/10 mg) | | (10 mg) | |
| Visit | n = 39 | | n = 75 | | n = 80 | |
| Base- | 84.4 | (9.21) | 81.2 | (9.72) | 81.4 | (10.24) |
| line | | | | | | |
| mean | | | | | | |
| (SD) | | | | | | |
| Week | 36 | | 63 | | 70 | |
| 1 | −3.9 | (−6.2, −1.5) | −5.5 | (−7.4, −3.5) | −2.9 | (−4.8, −0.9) |
| | −1.0 | (−3.6, 1.6) | −2.6 | (−4.7, −0.4) | | |
| Week | 35 | | 62 | | 71 | |
| 3 | −3.8 | (−6.7, −1.0) | −5.4 | (−7.7, −3.1) | −2.4 | (−4.6, −0.2) |
| | −1.4 | (−4.6, 1.8) | −2.9 | (−5.6, −0.3) | | |
| Week | 35 | | 56 | | 68 | |
| 6 | −4.5 | (−7.0, −2.0) | −7.4 | (−9.6, −5.3) | −2.7 | (−4.8, −0.7) |
| | =1.8 | (−4.6, 1.1) | −4.7 | (−7.1, −2.3) | | |
| Week | 31 | | 54 | | 65 | |
| 9 | −2.7 | (−5.3, −0.1) | −7.0 | (−9.1, −4.8) | −2.0 | (−4.1, 0.0) |
| | −0.6 | (−3.6, 2.3) | −4.9 | (−7.4, −2.5) | | |
| Week | 30 | | 46 | | 58 | |
| 12 | −5.9 | (−8.6, −3.2) | −5.9 | (−8.2, −3.6) | −2.2 | (−4.4, −0.1) |
| | −3.7 | (−6.7, −0.6)* | −3.7 | (−6.3, −1.0)** | | |

*p = 0.052,
**p = 0.024

### Example 4

Clinical trials of the fixed-dose combination of zibotentan and dapagliflozin are ongoing, including an international, multicenter, randomized, double-blind, active-controlled, parallel group study to assess the efficacy of a FDC of zibotentan and dapagliflozin compared to dapagliflozin alone (i.e., dapagliflozin monotherapy) in study participants having IgAN.

### Study Design

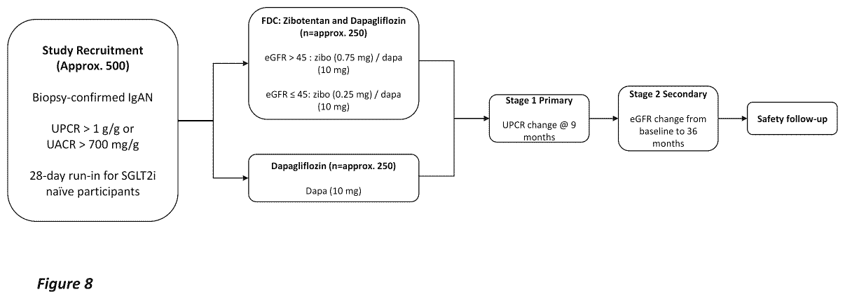

FIG. 8 provides an overview of the study. Approximately 500 participants will be randomized into one of two treatment arms in a 1:1 ratio: zibotentan (doses described below) and dapagliflozin (10 mg), once daily; dapagliflozin (10 mg), once daily. For participants receiving the FDC of zibotentan and dapagliflozin, the dose of zibotentan will be determined by eGFR status at the first screening visit: participants having an eGFR<45 mL/min/1.72 m² are administered zibotentan at dosage of 0.25 mg; participants having an eGFR ≥45 mL/min/1.72 m² are administered zibotentan at dosage of 0.75 mg. Study participants having an eGFR<45 mL/min/1.72 m² and ≥45 mL/min/1.72 m² will be stratified into the FDC (zibotentan and dapagliflozin) and dapagliflozin monotherapy treatment arms at a 1:1 ratio. Study participants will receive the blinded study intervention from Day 1 until Month 36. At the Month 36 visit, the participant will start open-label dapagliflozin therapy (10 mg). Participants who are not already on an SGLT2i at the first screening visit will receive a 28-day run-in intervention with an SGLT2i, once daily, prior to the start of the study.

Objectives for the study are broken into two stages: Stage 1 (9 months), and Stage 2 (36 months), and are detailed in Table 12.

TABLE 12
Study Objectives.

| | |
|---|---|
| Objectives | Estimands/Endpoints |

STAGE 1 (9 MONTHS)
Primary

| | |
|---|---|
| To determine whether zibotentan and | Change in UPCR vs dapagliflozin |
| dapagliflozin in FDC is superior to dapagliflozin | at 9 months |
| alone in reducing proteinuria | |

Exploratory

| | |
|---|---|
| To determine whether zibotentan and | Proportion of participants |
| dapagliflozin in FDC is superior to dapagliflozin | achieving |
| alone, in reducing proteinuria, as measured by | UPCR <1000 mg/g |
| the proportion of participants achieving | |
| UPCR <1000 mg/g reduction from baseline | |
| To determine whether zibotentan and | Proportion of participants |
| dapagliflozin in FDC is superior to dapagliflozin | achieving |
| alone, in reducing proteinuria, as measured by | UPCR <300 mg/g |
| the proportion of participants achieving | |
| UPCR <300 mg/g reduction from baseline | |
| To investigate the zibotentan and dapagliflozin | Pre-dose plasma concentrations |
| plasma exposure (PK) | of zibo and dapa |
| Safety: | Safety: |
| To assess the safety and tolerability of treatment | AEs, SAEs, SAEs with outcome |
| with zibotentan and dapagliflozin in FDC | of death, DAEs |
| compared to dapagliflozin alone. | Clinical laboratory/ |
| | Vital signs |
| | AESIs: heart failure, fluid |
| | retention, and hepatic events to |
| | evaluate hepatotoxicity |

STAGE 2 (36 MONTHS)
Secondary

| | |
|---|---|
| To determine whether zibotentan and | Change in eGFR from baseline at |
| dapagliflozin in FDC is superior to dapagliflozin | 36 months |
| alone to slow decline in kidney function | |
| To determine whether zibotentan and | Time to the first occurrence of any |
| dapagliflozin in FDC is superior to dapagliflozin | of the components of the renal |
| alone in reducing the incidence of the renal | composite endpoint of |
| composite endpoint of 40% sustained decline in | 40% sustained decline in eGFR or |
| eGFR or ESKD | ESKD |
| To determine whether zibotentan and | Change in systolic BP from |
| dapagliflozin in FDC is superior to dapagliflozin | baseline to each participant's |
| alone in reducing systolic BP. | mean level across the visits from |
| | Day 15 to Month 36 |

Exploratory

| | |
|---|---|
| Percent treated with rescue immunomodulatory | |
| medication (including steroids) | |
| PK (exploratory): To investigate the zibotentan | Pre-dose plasma concentrations |
| and dapagliflozin plasma exposure | of zibo and dapa |
| Safety: | Safety: |
| To assess the safety and tolerability of treatment | AEs, SAEs, SAEs with outcome |
| with zibotentan and dapagliflozin in FDC | of death, DAEs. |
| compared to dapagliflozin alone. | Clinical laboratory/ |
| | Vital signs |
| | AESIs: heart failure, fluid |
| | retention, and hepatic events to |
| | evaluate hepatotoxicity |

Key inclusion and exclusion criteria for the study are described below in Table 13.

TABLE 13
Study Inclusion and Exclusion Criteria.

Inclusion Criteria
Biopsy proven IgAN (at any time)
eGFR ≥30 mL/min/1.73 m²
UPCR ≥1 g/g or UACR ≥700 mg/g based on the screening value*
*A local laboratory value may be used instead if the following criteria apply: It must be
obtained within 4 months prior to screening Visit 1, and the participant must be in a
usual state of health without any modifications to RAASi (ACEi or ARB), MRA, or
SGLT2i therapy. Note: PER ≥1 g/day may be used for inclusion (instead of UPCR) if
PER is used locally for quantification.
Receiving RAASi therapy (ACEi or ARB), and for the patient maximum tolerated daily
dose, that has been stable for at least 4 weeks
Exclusion Criteria
NYHA III or IV at V1
hHF within 6 months prior to V1
Type I Diabetes Mellitus
Significant liver disease as judged by the investigator, or AST or ALT > 3 × ULN, or
TBL > 2 × ULN
Participants on systemic immunosuppression therapy for kidney disease other than
stable maintenance therapy defined as prednisone 10 mg/day (or equivalent) or less;
azathioprine 100 mg/day or less; mycophenolate mofetil 1000 mg/day (or equivalent);
inhaled, nasal, or dermatological steroids are also allowed
Participants treated or expecting to be treated with any other ERAs or orbudesonide
(where used to treat IBD or IgAN).
Participants treated with a strong or moderate CYP3A4 inducer or treated with a
strong CYP3A4 inhibitor

Study participant screening and randomization in the study will take place according to the Schedule of Activities set forth in Table 14A and 14B. Assessments during the study will take place according to the schedule of activities set forth in Table 15.

TABLE 14A
Screening and Randomization for SGLT2i Naïve Participants.

| | Procedure | | |
|---|---|---|---|
| | Screening ^{a} | | Randomization |
| | Visit | | |
| | 1 | 1.1 | 2 |
| | Day | | |
| | −34 (±5) ^{b} | −3 (±1) | 1 |
| | Month | | |
| | −1 | −1 | 0 |
| Informed consent | X | | |
| Informed consent addendum for | | X | |
| WOCBP | | | |
| Enrolment in IRT/RTSM | X | | |
| Demography | X | | |
| Inclusion and exclusion criteria | X | | |
| Medical/surgical history (includes | X | | |
| substance usage [and family history | | | |
| of premature CV disease]) | | | |
| Serum pregnancy test ^{c} | X | | |
| Urine pregnancy test and | | | X |
| contraception adherence check | | | |
| (WOCBP only) ^{d, e} | | | |
| FSH testing (central laboratory) ^{e} | X | | |
| Randomization in IRT/RTSM | | | X |
| Height | X | | |
| Vital signs (BP, pulse, and body | X | | X |
| weight) | | | |
| Full physical examination | X | | |
| Targeted physical examination ^{f} | | | X |
| NYHA functional classification | X | | |
| 12-lead ECG | | | X |
| Concomitant medication | X | | X |
| Serology (HIV I and II, hepatitis B | X | | |
| surface antigen, hepatitis C virus | | | |
| antibody, central laboratory) | | | |
| Random spot urine for UACR and | X | X | X ^{j} |
| UPCR: U-albumin, U-protein, and | | | |
| U-creatinine (central laboratory) ^{a} | | | |
| Clinical chemistry and haematology | X | | X |
| (central laboratory) ^{a, g} | | | |
| Additional blood sample for creatinine | | X | |
| (eGFR calculation) | | | |
| AEs including SAEs, DAEs, AESIs | X (SAEs | X (SAEs | X |
| | only) | only) | |
| EQ-5D-5L (including EQ-VAS) ^{h} | | | X |
| SPFQ (optional) ^{h} | | | X |
| Study intervention intake at the clinic | | | X |
| Dispense study intervention | | | X |
| Optional samples for future biomarker | | | X |
| research (plasma, serum, and urine) ⁱ | | | |
| Genomics Initiative optional, | | | X |
| exploratory genetic sample ⁱ | | | |

^{a} Individuals who do not meet the criteria for participation in this study (screen failure) may be fully re-screened once. Re-screened participants should re-sign informed consent and be assigned the same participant number as for the initial screening. Where the values for the following investigations are outside the usual range for a participant during screening, based on their medical history, re-testing may be undertaken on up to 2 occasions without requiring a re-screen: BP, eGFR, spot urine for UACR and UPCR, ALT, AST, and bilirubin.
^{b} The 10-day window (−34 [±5] days) for Visit 1 is to allow for turn-around time for central laboratory results before the 28-day run-in. Participants not already on a stable dose of SGLT2i will enter a 28-day run-in with SGLT2i from Day −28 through Day −1. During the run-in period, SGLT2i will be provided as an auxiliary drug.
^{c} Negative pregnancy test (serum) required for all women at the time of enrolment/prior to first dose of study intervention.
^{d} At Visit 2, urine pregnancy test result must be negative before the participant can be randomised. Monthly pregnancy test (urine) and contraception adherence check required for all WOCBP from randomization until 1 month after last dose. If positive, the treatment should be discontinued and procedures to be followed in the event of suspected contraceptive failure or suspicion of pregnancy. Options for pregnancy testing at home by a healthcare professional may be considered for months where no visit is scheduled.
^{e} Women not of childbearing potential are to be confirmed at screening by fulfilling one of the following criteria: (a) postmenopausal defined as amenorrhea for at least 12 months or more following cessation of all exogenous hormonal treatments and FSH levels in the postmenopausal range (Note: The postmenopausal range must be checked against the specific FSH assay used). In the absence of 12 months of amenorrhea, a single FSH measurement is insufficient to define postmenopausal criteria. In case of perimenopause or infrequent periods with variable levels of FSH, women should be considered of childbearing potential; or (b) documentation of irreversible surgical sterilisation by hysterectomy, bilateral oophorectomy, or bilateral salpingectomy but not tubal ligation.
^{f} Including signs of fluid retention and heart failure.
^{g} Tests to include: B-red blood cell count, B-haematocrit; B-haemoglobin, B-leukocyte differential count (absolute count), B-leukocyte count, B-platelet count, B-BNP, B-HbA1c, S-albumin, S-ALT, S-ALP, S-AST, S-bicarbonate, total S-bilirubin, S-BUN, total S-calcium, S-chloride, S-GGT, S-glucose, S-creatinine (including eGFR assessment), S-cystatin C, S-creatine kinase, S-magnesium, S-phosphorous, S-potassium, S-sodium.
^{h} PRO questionnaires should be completed prior to any other study procedures or discussions (following informed consent), including medication treatments, to avoid biasing the participant's responses to the questions.
ⁱ Participation in Genomics Initiative Research is optional and subject to separate consent by the participant.
^{j} At baseline, 2 samples for UPCR and UACR will be collected. One will be collected at home and one at the site.

TABLE 14B
Screening and Randomization for SGLT2i-Treated Participants.

| | Procedure | |
|---|---|---|
| | Screening ^{a, b} | Randomization |
| | Visit | |
| | 1 | 2 |
| | Day | |
| | −10 (±5) | 1 |
| | Month | |
| | −1 | 0 |
| Informed consent | X | |
| Informed consent addendum for | | X ^{c} |
| WOCBP | | |
| Enrolment in IRT/RTSM | X | |
| Demography | X | |
| Inclusion and exclusion criteria | X | |
| Medical/surgical history (includes | X | |
| substance usage [and family history of | | |
| premature CV disease]) | | |
| Serum pregnancy test ^{d} | X | |
| Urine pregnancy test and | | X |
| contraception adherence check | | |
| (WOCBP only) ^{e, f} | | |
| FSH testing (central laboratory) ^{f} | X | |
| Randomization in IRT/RTSM | | X |
| Height | X | |
| Vital signs (BP, pulse, and body | X | X |
| weight) | | |
| Full physical examination | X | |
| Targeted physical examination ^{g} | | X |
| NYHA functional classification | X | |
| 12-lead ECG | | X |
| Concomitant medication | X | X |
| Serology (HIV I and II, hepatitis B | X | |
| surface antigen, hepatitis C virus | | |
| antibody, central laboratory) | | |
| Random spot urine for UACR and | X | X ^{k} |
| UPCR: U-albumin, U-protein, and | | |
| U-creatinine (central laboratory) | | |
| Clinical chemistry and haematology | X | X |
| (central laboratory) ^{h} | | |
| AEs including SAEs, DAEs, AESIs | X (SAEs only) | X |
| EQ-5D-5L (including EQ-VAS) ⁱ | | X |
| SPFQ (optional) ⁱ | | X |
| Study intervention intake at clinic | | X |
| Dispense study intervention | | X |
| Optional samples for future biomarker | | X |
| research (plasma, serum, and urine) | | |
| Genomics Initiative optional, | | X |
| exploratory genetic sample ^{j} | | |

^{a} Individuals who do not meet the criteria for participation in this study (screen failure) may be fully re-screened once. Re-screened participants should re-sign informed consent and be assigned the same participant number as for the initial screening. Where the values for the following investigations are outside the usual range for a participant during screening, based on their medical history, re-testing may be undertaken on up to 2 occasions without requiring a re-screen: BP, eGFR, spot urine for UACR and UPCR, ALT, AST, and bilirubin.
^{b} Participants already on a stable dose of SGLT2i.
^{c} At the latest signed prior to randomization once it is confirmed that the participant is of childbearing potential.
^{d} Negative pregnancy test (serum) required for all women at the time of enrolment/prior to first dose of study intervention.
^{e} At Visit 2, urine pregnancy test result must be negative before the participant can be randomised. Monthly pregnancy test (urine) and contraception adherence check required for all WOCBP from randomization until 1 month after last dose. If positive, the treatment should be discontinued and procedures to be followed in the event of suspected contraceptive failure or suspicion of pregnancy. Options for pregnancy testing at home by a healthcare professional may be considered for months where no visit is scheduled.
^{f} Women not of childbearing potential are to be confirmed at screening by fulfilling one of the following criteria: (a) postmenopausal defined as amenorrhea for at least 12 months or more following cessation of all exogenous hormonal treatments and FSH levels in the postmenopausal range (Note: The postmenopausal range must be checked against the specific FSH assay used). In the absence of 12 months of amenorrhea, a single FSH measurement is insufficient to define postmenopausal criteria. In case of perimenopause or infrequent periods with variable levels of FSH, women should be considered of childbearing potential; or (b) documentation of irreversible surgical sterilisation by hysterectomy, bilateral oophorectomy, or bilateral salpingectomy but not tubal ligation.
^{g} Including signs of fluid retention and heart failure.
^{h} Tests to include: B-red blood cell count, B-haematocrit; B-haemoglobin, B-leukocyte differential count (absolute count), B-leukocyte count, B-platelet count, B-BNP, B-HbA1c, S-albumin, S-ALT, S-ALP, S-AST, S-bicarbonate, total S-bilirubin, S-BUN, total S-calcium, S-chloride, S-GGT, S-glucose, S-creatinine (including eGFR assessment), S-cystatin C, S-creatine kinase, S-magnesium, S-phosphorous, S-potassium, S-sodium.
ⁱ PRO questionnaires should be completed prior to any other study procedures or discussions (following informed consent), including medication treatments, to avoid biasing the participant's responses to the questions.
^{j} Participation in Genomics Initiative Research is optional and subject to separate consent by the participant.
^{k} At baseline, 2 samples for UACR and UACR will be collected. One will be collected at home and one at the site.

TABLE 15
Schedule of Activities.

| | Intervention Period | | | | | | | | | |
|---|---|---|---|---|---|---|---|---|---|---|
| | Visit | | | | | | | | Post-treatment | |
| | | | | | | | 9, 10, 11, | | discontinuation | |
| | 3 | 4 ^{b} | 5 | 6 | 7 | 8 | 12, 13, 14 | 14.1 | visit | Safety Visit |
| | Day | | | | | | | | | |
| | | | | | | | 480 (±10), | | | |
| | | | | | | | 600 (± 10), | | | |
| | | | | | | | 720 (±7), | 7 (±3) | Within 28 | 30 (±3) |
| | 15 | 30 | 90 | 180 | 270 | 360 | then Q4M | days after | days after | days after |
| | (±3) | (±3) | (±7) | (±7) | (±7) | (±7) | (±7 days) | Visit 11 | last dose | V14.1 or PTDV |
| | Month | | | | | | | | | |
| | | | | | | | 16, 20, 24, | 36 + 1 | | |
| | 0.5 | 1 | 3 | 6 | 9 | 12 | 28, 32, 36 | week | | |
| Urine pregnancy test | | X (monthly) | | | | | | | X | X |
| and contraception | | | | | | | | | | |
| adherence check (WOCBP | | | | | | | | | | |
| only) ^{c, d} | | | | | | | | | | |
| Vital signs (BP, pulse, | X | | X | X | X | X | X ^{m} | X | X | X |
| and body weight) | | | | | | | | | | |
| Full physical | | | | | | | | | X | |
| examination | | | | | | | | | | |
| Targeted physical | X | | X | X | X | X | X ^{m} | X | | |
| examination ^{e} | | | | | | | | | | |
| 12-lead ECG | | | | X | | X | X (yearly) ^{m} | | X | |
| Concomitant medication | X | | X | X | X | X | X ^{m} | X | X | X |
| Random spot urine for | X | | X | X | X ^{l} | X | X ^{m} | X | X | X |
| UACR and UPCR: U-albumin, | | | | | | | | | | |
| U-protein, and U-creatinine | | | | | | | | | | |
| (central laboratory) | | | | | | | | | | |
| Clinical chemistry and | X | | X | X | X | X | X ^{m} | X | X | X |
| haematology (central | | | | | | | | | | |
| laboratory) ^{f} | | | | | | | | | | |
| PK sampling (pre-dose) ^{h} | X | | X | X | | | X (Visit | | | |
| | | | | | | | 12 only) | | | |
| AEs including SAEs, DAEs, | X | X | X | X | X | X | X | X | X | X |
| AESIs | | | | | | | | | | |
| EQ-5D-5L (including EQ-VAS) ⁱ | | | | X | | X | | X | X | |
| SPFQ (optional) ⁱ | | | | | | | | X | X ^{j} | |
| Dispense study intervention | X | | X | X | X | X | X | X ^{a} | | |
| Accountability (study | X | | X | X | X | X | X ^{m} | X | X | |
| intervention compliance | | | | | | | | | | |
| reminder, collect unused | | | | | | | | | | |
| study intervention) | | | | | | | | | | |
| Study intervention | X | | | X | | | X (Visit | | | |
| intake at clinic ^{k} | | | | | | | 12 only) | | | |
| Optional samples for | | | | | | X | | X | | |
| future biomarker research | | | | | | | | | | |
| (plasma, serum and urine) | | | | | | | | | | |

^{a} At Visit 14.1, participants still on blinded treatment will discontinue blinded study intervention and start open-label monotherapy with dapagliflozin 10 mg QD provided as an auxiliary drug until the safety follow-up visit. For participants who have prematurely discontinued study intervention but remained in the study as per the SoA, visit 14.1 will be the final study visit.
^{b} Visit 4: phone call to assess fluid retention and related conditions.
^{c} Monthly pregnancy test (urine) and contraception adherence check required for all WOCBP from randomization until 1 month after last dose. If positive, the treatment should be discontinued and procedures to be followed in the event of suspected contraceptive failure or suspicion of pregnancy. The company may consider options for pregnancy testing at home by a health care professional for months where no visit is scheduled.
^{d} Women not of childbearing potential are to be confirmed at screening by fulfilling one of the following criteria: (a) postmenopausal defined as amenorrhea for at least 12 months or more following cessation of all exogenous hormonal treatments and FSH levels in the postmenopausal range (in the absence of 12 months of amenorrhea, a single FSH measurement is insufficient to define postmenopausal criteria. In case of perimenopause or infrequent periods with variable levels of FSH, women should be considered of childbearing potential); or (b) documentation of irreversible surgical sterilisation by hysterectomy, bilateral oophorectomy, or bilateral salpingectomy but not tubal ligation.
^{e} Including signs of fluid retention and heart failure.
^{f} Tests to include: B-red blood cell count, B-haematocrit, B-haemoglobin, B-leukocyte differential count (absolute count), B-leukocyte count, B-platelet count, B-BNP, B-HbA1c, S-albumin, S-ALT, S-ALP, S-AST, S-bicarbonate, total S-bilirubin, S-BUN, total S-calcium, S-chloride, S-GGT, S-glucose, S-creatinine (including eGFR assessment), S-cystatin C, S-creatine kinase, S-magnesium, S-phosphorous, S-potassium, S-sodium.
^{g} Visit 14 mean eGFR value will be calculated from eGFR values at Visit 14 and Visit 14.1.
^{h} PK samples will be collected pre-dose at Visits 3, 5, 6, and 12. In participants who have permanently discontinued study intervention, PK samples should not be collected if more than 7 days have passed since the last dosing.
ⁱ PRO questionnaires should be completed prior to any other study procedures or discussions (following informed consent), including medication treatments, to avoid biasing the participant's responses to the questions.
^{j} If possible, the remaining (previously uncompleted) sections of the SPFQ should be completed at the PTDV only if the participant withdraws early from the study.
^{k} Outside of Visits 3, 5, 6, and 12, study intervention will be taken at home.
^{l} At visit 6 (6 months) a tube to collect urine will be dispensed in order to have 2 samples for UACR and UACR at Visit 7 (9 months). One will be collected at home and one at the site. At visits other than screening, randomization and 9 months, only one urine sample will be collected, at site.
^{m} Except for visit 14

1-84. (canceled)

85. A method of treating chronic kidney disease in a human patient who has:
a) a urinary protein to creatine ratio (UPCR) of greater than 1 g/g, or
b) a urinary albumin to creatine ratio (UACR) of greater than 700 mg/g, the method comprising administering to the patient a fixed-dose combination of zibotentan and dapagliflozin in an amount effective to treat the patient's chronic kidney disease.

86. A method of slowing decline in renal function in a human patient who has:
a) a urinary protein to creatine ratio (UPCR) of greater than 1 g/g, or
b) a urinary albumin to creatine ratio (UACR) of greater than 700 mg/g, the method comprising administering to the patient a fixed-dose combination of zibotentan and dapagliflozin in an amount effective to slow the patient's decline in renal function relative to a dosing regimen in which the patient receives dapagliflozin alone.

87. A method of reducing proteinuria in a human patient who has:
a) a urinary protein to creatine ratio (UPCR) of greater than 1 g/g, or
b) a urinary albumin to creatine ratio (UACR) of greater than 700 mg/g, the method comprising administering to the patient a fixed-dose combination of zibotentan and dapagliflozin in an amount effective to reduce the patient's proteinuria relative to a dosing regimen in which the patient receives dapagliflozin alone.

88. A method of reducing albuminuria in a human patient who has:
a) a urinary protein to creatine ratio (UPCR) of greater than 1 g/g, or
b) a urinary albumin to creatine ratio (UACR) of greater than 700 mg/g, the method comprising administering to the patient a fixed-dose combination of zibotentan and dapagliflozin in an amount effective to reduce the patient's albuminuria relative to a dosing regimen in which the patient receives dapagliflozin alone.

89. The method of claim 85, wherein the patient has an estimated glomerular filtration rate (eGFR) of <90 mL/min/1.73 m²

90. The method of claim 89, wherein the patient has an estimated glomerular filtration rate (eGFR) of ≥20-<90 mL/min/1.73 m².

91. The method of claim 85, wherein the patient has a UPCR of 1-1.3 g/g.

92. The method of claim 85, wherein the patient has a UACR of 700-900 mg/g.

93. The method of claim 85, wherein the patient has a UPCR of 1-1.3 g/g and a UACR of 700-900 mg/g.

94. The method of claim 85, comprising administering the fixed-dose combination of zibotentan and dapagliflozin to the patient once per day.

95. The method of claim 85, comprising administering zibotentan at a dose of 0.25 mg to 1.5 mg.

96. The method of claim 85, comprising administering dapagliflozin at a dose of 10.0 mg.

97. The method of claim 85, comprising administering zibotentan at a dose of 0.25 mg and dapagliflozin at a dose of 10 mg when the patient has an eGFR that is <45 mL/min/1.73 m², and administering zibotentan at a dose of 0.75 mg and dapagliflozin at a dose of 10 mg when the patient has an eGFR that is ≥45 mL/min/1.73 m².

98. The method of claim 97, wherein the fixed dose combination of zibotentan and dapagliflozin is adjusted from 0.75 mg of zibotentan and 10 mg of dapagliflozin to 0.25 mg of zibotentan and 10 mg of dapagliflozin when the patient's eGFR changes to <45 mL/min/1.73 m².

99. The method of claim 97, wherein the fixed dose combination of zibotentan and dapagliflozin is adjusted from 0.25 mg of zibotentan and 10 mg of dapagliflozin to 0.75 mg of zibotentan and 10 mg of dapagliflozin when the patient's eGFR changes to ≥45 mL/min/1.73 m².

100. The method of claim 85, wherein administration of the fixed-dose combination of zibotentan and dapagliflozin reduces the patient's UACR to below 300 mg/g.

101. The method of claim 85, wherein administration of the fixed-dose combination of zibotentan and dapagliflozin reduces the patient's UPCR to below 1 g/g.

102. The method of claim 85, wherein administration of the fixed-dose combination of zibotentan and dapagliflozin reduces the patient's UACR to below 300 mg/g and the patient's UPCR to below 1 g/g.

103. The method of claim 85, wherein the patient achieves partial remission or remission.

104. The method of claim 85, wherein administration of the fixed-dose combination of zibotentan and dapagliflozin reduces incidence of stroke and/or acute coronary syndrome in the patient relative to a dosing regimen in which the patient receives dapagliflozin alone.

105. The method of claim 85, wherein administration of the fixed-dose combination of zibotentan and dapagliflozin does not lead to hospitalization of heart failure for the patient.

106. The method of claim 85, wherein administration of the fixed-dose combination of zibotentan and dapagliflozin reduces the risk of hospitalization of heart failure in the patient relative to a dosing regimen in which the patient receives dapagliflozin alone.

107. The method of claim 85, wherein administration of the fixed-dose combination of zibotentan and dapagliflozin reduces the patient's blood pressure relative to a dosing regimen in which the patient receives dapagliflozin alone.

108. The method of claim 85, wherein administration of the fixed-dose combination of zibotentan and dapagliflozin reduces the patient's blood pressure relative to a dosing regimen in which the patient receives dapagliflozin alone, and wherein the patient's blood pressure remains reduced relative to the dosing regimen in which the patient receives dapagliflozin alone for at least two weeks after administration of the fixed-dose combination of zibotentan and dapagliflozin is stopped.

109. The method of claim 85, wherein administration of the fixed-dose combination of zibotentan and dapagliflozin reduces the risk of blood pressure elevation in the patient relative to a dosing regimen in which the patient receives dapagliflozin alone.

110. The method of claim 85, wherein administration of the fixed-dose combination of zibotentan and dapagliflozin does not increase the patient's fluid retention relative to a dosing regimen in which the patient receives dapagliflozin alone.

111. The method of claim 85, wherein administration of the fixed-dose combination of zibotentan and dapagliflozin reduces the risk of increased fluid retention in the patient relative to a dosing regimen in which the patient receives dapagliflozin alone.

112. The method of claim 85, wherein administration of the fixed-dose combination of zibotentan and dapagliflozin reduces proteinuria in the patient relative to a dosing regimen in which the patient receives dapagliflozin alone.

113. The method of claim 85, wherein administration of the fixed-dose combination of zibotentan and dapagliflozin reduces the rate of kidney function decline in the patient relative to a dosing regimen in which the patient receives dapagliflozin alone.
